SEQUENCE LISTING (245 sequences)

SEQ ID NO: 1 - protein, 832 aa, Homo Sapiens
  MISC_FEATURE (1)..(832): Accession No Q12864
MILQAHLHSLCLLMLYLATGYGQEGKFSGPLKPMTFSIYEGQEPSQIIFQFKANPPAVTFELTGETDNIFVIEREGLLYYNRALDRETRSTHNLQVAALDANGIIVEGPVPITIKVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGMAVGILLTTLLVIGIILAVVFIRIKKDKGKDNVESAQASEVKPLRS

SEQ ID NO: 2 - protein, 319 aa, Homo Sapiens
  MISC_FEATURE (1)..(319): Accession No Q99795
MVGKMWPVLWTLCAVRVTVDAISVETPQDVLRASQGKSVTLPCTYHTSTSSREGLIQWDKLLLTHTERVVIWPFSNKNYIHGELYKNRVSISNNAEQSDASITIDQLTMADNGTYECSVSLMSDLEGNTKSRVRLLVLVPPSKPECGIEGETIIGNNIQLTCQSKEGSPTPQYSWKRYNILNQEQPLAQPASGQPVSLKNISTDTSGYYICTSSNEEGTQFCNITVAVRSPSMNVALYVGIAVGVVAALIIIGIIIYCCCCRGKDDNTEDKEDARPNREAYEEPPEQLRELSREREEEDDYRQEEQRSTGRESPDHLDQ

SEQ ID NO: 3 - protein, 1055 aa, Homo Sapiens
  MISC_FEATURE (1)..(1055): Accession No P29323
MALRRLGAALLLLPLLAAVEETLMDSTTATAELGWMVHPPSGWEEVSGYDENMNTIRTYQVCNVFESSQNNWLRTKFIRRRGAHRIHVEMKFSVRDCSSIPSVPGSCKETFNLYYYEADFDSATKTFPNWMENPWVKVDTIAADESFSQVDLGGRVMKINTEVRSFGPVSRSGFYLAFQDYGGCMSLIAVRVFYRKCPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDLVYNIICKSCGSGRGACTRCGDNVQYAPRQLGLTEPRIYISDLLAHTQYTFEIQAVNGVTDQSPFSPQFASVNITTNQAAPSAVSIMHQVSRTVDSITLSWSQPDQPNGVILDYELQYYEKELSEYNATAIKSPTNTVTVQGLKAGAIYVFQVRARTVAGYGRYSGKMYFQTMTEAEYQTSIQEKLPLIIGSSAAGLVFLIAVVVIAIVCNRRGFERADSEYTDKLQHYTSGHMTPGMKIYIDPFTYEDPNEAVREFAKEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPNSLKAMAPLSSGINLPLLDRTIPDYTSFNTVDEWLEAIKMGQYKESFANAGFTSFDVVSQMMMEDILRVGVTLAGHQKKILNSIQVMRAQMNQIQSVEGQPLARRPRATGRTKRCQPRDVTKKTCNSNDGKKKGMGKKKTDPGRGREIQGIFFKEDSHKESNDCSCGG

SEQ ID NO: 4 - protein, 855 aa, Homo Sapiens [sequence omitted]

SEQ ID NO: 5 - protein, 802 aa, Homo Sapiens
  MISC_FEATURE (1)..(802): Accession No P18433
MDSWFILVLLGSGLICVSANNATTVAPSVGITRLINSSTAEPVKEEAKTSNPTSSLTSLSVAPTFSPNITLGPTYLTTVNSSDSDNGTTRTASTNSIGITISPNGTWLPDNQFTDARTEPWEGNSSTAATTPETFPPSDETPIIAVMVALSSLLVIVFIIIVLYMLRFKKYKQAGSHSNSKQAGSHSNSFRLSNGRTEDVEPQSVPLLARSPSTNRKYPPLPVDKLEEEINRRMADDNKLFREEFNALPACPIQATCEAASKEENKEKNRYVNILPYDHSRVHLTPVEGVPDSDYINASFINGYQEKNKFIAAQGPKEETVNDFWRMIWEQNTATIVMVTNLKERKECKCAQYWPDQGCWTYGNIRVSVEDVTVLVDYTVRKFCIQQVGDMTNRKPQRLITQFHFTSWPDFGVPFTPIGMLKFLKKVKACNPQYAGAIVVHCSAGVGRTGTFVVIDAMLDMMHTERKVDVYGFVSRIRAQRCQMVQTDMQYVFIYQALLEHYLYGDTELEVTSLETHLQKIYNKIPGTSNNGLEEEFKKLTSIKIQNDKMRTGNLPANMKKNRVLQIIPYEFNRVIIPVKRGEENTDYVNASFIDGYRQKDSYIASQGPLLHTIEDFWRMIWEWKSCSIVMLTELEERGQEKCAQYWPSDGLVSYGDITVELKKEEECESYTVRDLLVTNTRENKSRQIRQFHFHGWPEVGIPSDGKGMISIIAAVQKQQQQSGNHPITVHCSAGAGRTGTFCALSTVLERVKAEGILDVFQTVKSLRLQRPHMVQTLEQYEFCYKVVQEYIDAFSDYANFK

SEQ ID NO: 6 - protein, 1015 aa, Homo Sapiens
  MISC_FEATURE (1)..(1015): Accesssion No Q6PIM3
MRRFLRPGHDPVRERLKRDLFQFNKTVEHGFPHQPSALGYSPSLRILAIGTRSGAIKLYGAPGVEFMGLHQENNAVTQIHLLPGQCQLVTLLDDNSLHLWSLKVKGGASELQEDESFTLRGPPGAAPSATQITVVLPHSSCELLYLGTESGNVFVVQLPAFRALEDRTISSDAVLQRLPEEARHRRVFEMVEALQEHPRDPNQILIGYSRGLVVIWDLQGSRVLYHFLSSQQLENIWWQRDGRLLVSCHSDGSYCQWPVSSEAQQPEPLRSLVPYGPFPCKAITRILWLTTRQGLPFTIFQGGMPRASYGDRHCISVIHDGQQTAFDFTSRVIGFTVLTEADPAATFDDPYALVVLAEEELVVIDLQTAGWPPVQLPYLASLHCSAITCSHHVSNIPLKLWERIIAAGSRQNAHFSTMEWPIDGGTSLTPAPPQRDLLLTGHEDGTVRFWDASGVCLRLLYKLSTVRVFLTDTDPNENFSAQGEDEWPPLRKVGSFDPYSDDPRLGIQKIFLCKYSGYLAVAGTAGQVLVLELNDEAAEQAVEQVEADLLQDQEGYRWKGHERLAARSGPVRFEPGFQPFVLVQCQPPAVVTSLALHSEWRLVAFGTSHGFGLFDHQQRRQVFVKCTLHPSDQLALEGPLSRVKSLKKSLRQSFRRMRRSRVSSRKRHPAGPPGEAQEGSAKAERPGLQNMELAPVQRKIEARSAEDSFTGFVRTLYFADTYLKDSSRHCPSLWAGTNGGTIYAFSLRVPPAERRMDEPVRAEQAKEIQLMHRAPVVGILVLDGHSVPLPEPLEVAHDLSKSPDMQGSHQLLVVSEEQFKVFTLPKVSAKLKLKLTALEGSRVRRVSVAHFGSRRAEDYGEHHLAVLTNLGDIQVVSLPLLKPQVRYSCIRREDVSGIASCVFTKYGQGFYLISPSEFERFSLSTKWLVEPRCLVDSAETKNHRPGNGAGPKKAPSRARNSGTQSDGEEKQPGLVMERALLSDERAATGVHIEPPWGAASAMAEQSEWLSVQAAR

SEQ ID NO: 7 - protein, 166 aa, Homo Sapiens
  MISC_FEATURE (1)..(166): Accession No Q8TD06
MMLHSALGLCLLLVTVSSNLAIAIKKEKRPPQTLSRGWGDDITWVQTYEEGLFYAQKSKKPLMVIHHLEDCQYSQALKKVFAQNEEIQEMAQNKFIMLNLMHETTDKNLSPDGQYVPRIMFVDPSLTVRADIAGRYSNRLYTYEPRDLPLLIENMKKALRLIQSEL

SEQ ID NO: 8 - protein, 801 aa, Homo Sapiens
  MISC_FEATURE (1)..(801): Accession No Q9UN66
MEASGKLICRQRQVLFSFLLLGLSLAGAAEPRSYSVVEETEGSSFVTNLAKDLGLEQREFSRRGVRVVSRGNKLHLQLNQETADLLLNEKLDREDLCGHTEPCVLRFQVLLESPFEFFQAELQVIDINDHSPVFLDKQMLVKVSESSPPGTAFPLKNAEDLDIGQNNIENYIISPNSYFRVLTRKRSDGRKYPELVLDNALDREEEAELRLTLTALDGGSPPRSGTAQVYIEVVDVNDNAPEFQQPFYRVQISEDSPISFLVVKVSATDVDTGVNGEISYSLFQASDEISKTFKVDFLTGEIRLKKQLDFEKFQSYEVNIEARDAGGFSGKCTVLIQVIDVNDHAPEVTMSAFTSPIPENAPETVVALFSVSDLDSGENGKISCSIQEDLPFLLKSSVGNFYTLLTETPLDRESRAEYNVTITVTDLGTPRLTTHLNMTVLVSDVNDNAPAFTQTSYTLFVRENNSPALHIGSVSATDRDSGTNAQVTYSLLPPQDPHLPLASLVSINTDNGHLFALRSLDYEALQAFEFRVGASDRGSPALSSEALVRVLVLDANDNSPFVLYPLQNGSAPCTELVPRAAEPGYLVTKVVAVDGDSGQNAWLSYQLLKATEPGLFGVWAHNGEVRTARLLSERDAAKQRLVVLVKDNGEPPCSATATLHLLLVDGFSQPYLPLPEAAPAQGQADSLTVYLVVALASVSSLFLFSVLLFVAVLLCRRSRAASVGRCSVPEGPFPGHLVDVRGTGSLSQNYQYEVCLAGGSGTNEFQFLKPVLPNIQGHSFGPEMEQNSNFRNGFGFSLQLK

SEQ ID NO: 9 - protein, 314 aa, Homo Sapiens
  MISC_FEATURE (1)..(314): Accession No P16422
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 10 - protein, 768 aa, Homo Sapiens
  MISC_FEATURE (1)..(768): Accession No ENST00000322765
MLCGRWRRCRRPPEEPPVAAQVAAQVAAPVALPSPPTPSDGGTKRPGLRALKKMGLTEDEDVRAMLRGSRLRKIRSRTWHKERLYRLQEDGLSVWFQRRIPRAPSQHIFFVQHIEAVREGHQSEGLRRFGGAFAPARCLTIAFKGRRKNLDLAAPTAEEAQRWVRASYLRAGGSLACCCYFLSTHTWIHSYLHRADSNQDSKMSFKEIKSLLRMVNVDMNDMYAYLLFKECDHSNNDRLEGAEIEEFLRRLLKRPELEEIFHQYSGEDRVLSAPELLEFLEDQGEEGATLARAQQLIQTYELNETAKQHELMTLDGFMMYLLSPEGAALDNTHTCVFQDMNQPLAHYFISSSHNTYLTDSQIGGPSSTEAYVRAFAQGCRCVELDCWEGPGGEPVIYHGHTLTSKILFRDVVQAVRDHAFTLSPYPVILSLENHCGLEQQAAMARHLCTILGDMLVTQALDSPNPEELPSPEQLKGRVLVKGKKLPAARSEDGRALSDREEEEEDDEEEEEEVEAAAQRRLLHPAPNAPQPCQVSSLSERKAKKLIREAGNSFVRHNARQLTRVYPLGLRMNSANYSPQEMWNSGCQLVALNFQTPGYEMDLNAGRFLVNGQCGYVLKPACLRQPDSTFDPEYPGPPRTTLSIQVLTAQQLPKLNAEKPHSIVDPLVRIEIHGVPADCARQETDYVLNNGFNPRWGQTLQFQLRAPELALVRFVVEDYDATSPNDFVGQFTLPLSSLKQGYRHIHLLSKDGASLSPATLFIQIRIQRS

SEQ ID NO: 11 - protein, 517 aa, Homo Sapiens
  MISC_FEATURE (1)..(517): Accession No O00515
MAVSRKDWSALSSLARQRTLEDEEEQERERRRRHRNLSSTTDDEAPRLSQNGDRQASASERLPSVEEAEVPKPLPPASKDEDEDIQSILRTRQERRQRRQVVEAAQAPIQERLEAEEGRNSLSPVQATQKPLVSKKELEIPPRRRLSREQRGPWPLEEESLVGREPEERKKGVPEKSPVLEKSSMPKKTAPEKSLVSDKTSISEKVLASEKTSLSEKIAVSEKRNSSEKKSVLEKTSVSEKSLAPGMALGSGRRLVSEKASIFEKALASEKSPTADAKPAPKRATASEQPLAQEPPASGGSPATTKEQRGRALPGKNLPSLAKQGASDPPTVASRLPPVTLQVKIPSKEEEADMSSPTQRTYSSSLKRSSPRTISFRMKPKKENSETTLTRSASMKLPDNTVKLGEKLERYHTAIRRSESVKSRGLPCTELFVAPVGVASKRHLFEKELAGQSRAEPASSRKENLRLSGVVTSRLNLWISRTQESGDQDPQEAQKASSATERTQWGQKSDSSLDAEV

SEQ ID NO: 12 - protein, 733 aa, Homo Sapiens
  MISC_FEATURE (1)..(733): Accession No Q96TA1
MGWMGEKTGKILTEFLQFYEDQYGVALFNSMRHEIEGTGLPQAQLLWRKVPLDERIVFSGNLFQHQEDSKKWRNRFSLVPHNYGLVLYENKAAYERQVPPRAVINSAGYKILTSVDQYLELIGNSLPGTTAKSGSAPILKCPTQFPLILWHPYARHYYFCMMTEAEQDKWQAVLQDCIRHCNNGIPEDSKVEGPAFTDAIRMYRQSKELYGTWEMLCGNEVQILSNLVMEELGPELKAELGPRLKGKPQERQRQWIQISDAVYHMVYEQAKARFEEVLSKVQQVQPAMQAVIRTDMDQIITSKEHLASKIRAFILPKAEVCVRNHVQPYIPSILEALMVPTSQGFTEVRDVFFKEVTDMNLNVINEGGIDKLGEYMEKLSRLAYHPLKMQSCYEKMESLRLDGLQQRFDVSSTSVFKQRAQIHMREQMDNAVYTFETLLHQELGKGPTKEELCKSIQRVLERVLKKYDYDSSSVRKRFFREALLQISIPFLLKKLAPTCKSELPRFQELIFEDFARFILVENTYEEVVLQTVMKDILQAVKEAAVQRKHNLYRDSMVMHNSDPNLHLLAEGAPIDWGEEYSNSGGGGSPSPSTPESATLSEKRRRAKQVVSVVQDEEVGLPFEASPESPPPASPDGVTEIRGLLAQGLRPESPPPAGPLLNGAPAGESPQPKAAPEASSPPASPLQHLLPGKAVDLGPPKPSDQETGEQVSSPSSHPALHTTTEDSAGVQTEF

SEQ ID NO: 13 - protein, 175 aa, Homo Sapiens
  MISC_FEATURE (1)..(175): Accession No O95994
MEKIPVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 14 - protein, 1383 aa, Homo Sapiens
  MISC_FEATURE (1)..(1383): Accession No Q9UHN6
MYATDSRGHSPAFLQPQNGNSRHPSGYVPGKVVPLRPPPPPKSQASAKFTSIRREDRATFAFSPEEQQAQRESQKQKRHKNTFICFAITSFSFFIALAIILGISSKYAPDENCPDQNPRLRNWDPGQDSAKQVVIKEGDMLRLTSDATVHSIVIQDGGLLVFGDNKDGSRNITLRTHYILIQDGGALHIGAEKCRYKSKATITLYGKSDEGESMPTFGKKFIGVEAGGTLELHGARKASWTLLARTLNSSGLPFGSYTFEKDFSRGLNVRVIDQDTAKILESERFDTHEYRNESRRLQEFLRFQDPGRIVAIAVGDSAAKSLLQGTIQMIQERLGSELIQGLGYRQAWALVGVIDGGSTSCNESVRNYENHSSGGKALAQREFYTVDGQKFSVTAYSEWIEGVSLSGFRVEVVDGVKLNLLDDVSSWKPGDQIVVASTDYSMYQAEEFTLLPCSECSHFQVKVKETPQFLHMGEIIDGVDMRAEVGILTRNIVIQGEVEDSCYAENQCQFFDYDTFGGHIMIMKNFTSVHLSYVELKHMGQQQMGRYPVHFHLCGDVDYKGGYRHATFVDGLSIHHSFSRCITVHGTNGLLIKDTIGFDTLGHCFFLEDGIEQRNTLFHNLGLLTKPGTLLPTDRNNSMCTTMRDKVFGNYIPVPATDCMAVSTFWIAHPNNNLINNAAAGSQDAGIWYLFHKEPTGESSGLQLLAKPELTPLGIFYNNRVHSNFKAGLFIDKGVKTTNSSAADPREYLCLDNSARFRPHQDANPEKPRVAALIDRLIAFKNNDNGAWVRGGDIIVQNSAFADNGIGLTFASDGSFPSDEGSSQEVSESLFVGESRNYGFQGGQNKYVGTGGIDQKPRTLPRNRTFPIRGFQIYDGPIHLTRSTFKKYVPTPDRYSSAIGFLMKNSWQITPRNNISLVKFGPHVSLNVFFGKPGPWFEDCEMDGDKNSIFHDIDGSVTGYKDAYVGRMDNYLIRHPSCVNVSKWNAVICSGTYAQVYVQTWSTQNLSMTITRDEYPSNPMVLRGINQKAAFPQYQPVVMLEKGYTIHWNGPAPRTTFLYLVNFNKNDWIRVGLCYPSNTSFQVTFGYLQRQNGSLSKIEEYEPVHSLEELQRKQSERKFYFDSSTGLLFLYLKAKSHRHGHSYCSSQGCERVKIQAATDSKDISNCMAKAYPQYYRKPSVVKRMPAMLTGLCQGCGTRQVVFTSDPHKSYLPVQFQSPDKAETQRGDPSVISVNGTDFTFRSAGVLLLVVDPCSVPFRLTEKTVFPLADVSRIEEYLKTGIPPRSIVLLSTRGEIKQLNISHLLVPLGLAKPAHLYDKGSTIFLGFSGNFKPSWTKLFTSPAGQGLGVLEQFIPLQLDEYGCPRATTVRRRDLELLKQASKAH

SEQ ID NO: 15 - protein, 764 aa, Homo Sapiens
  MISC_FEATURE (1)..(764): Accession No P01833
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 16 - protein, 318 aa, Homo Sapiens
  MISC_FEATURE (1)..(318): Accession No Q92820
MASPGCLLCVLGLLLCGAASLELSRPHGDTAKKPIIGILMQKCRNKVMKNYGRYYIAASYVKYLESAGARVVPVRLDLTEKDYEILFKSINGILFPGGSVDLRRSDYAKVAKIFYNLSIQSFDDGDYFPVWGTCLGFEELSLLISGECLLTATDTVDVAMPLNFTGGQLHSRMFQNFPTELLLSLAVEPLTANFHKWSLSVKNFTMNEKLKKFFNVLTTNTDGKIEFISTMEGYKYPVYGVQWHPEKAPYEWKNLDGISHAPNAVKTAFYLAEFFVNEARKNNHHFKSESEEEKALIYQFSPIYTGNISSFQQCYIFD

SEQ ID NO: 17 - protein, 315 aa, Homo Sapiens
  MISC_FEATURE (1)..(315): Accession No P27216
GNRHAKASSPQGFDVDRDAKKLNKACKGMGTNEAAIIEILSGRTSDERQQIKQKYKATYGKELEEVLKSELSGNFEKTALALLDRPSEYAARQLQKAMKGLGTDESVLIEFLCTRTNKEIIAIKEAYQRLFDRSLESDVKGDTSGNLKKILVSLLQANRNEGDDVDKDLAGQDAKDLYDAGEGRWGTDELAFNEVLAKRSYKQLRATFQAYQILIGKDIEEAIEEETSGDLQKAYLTLVRCAQDCEDYFAERLYKSMKGAGTDEETLIRIVVTRAEVDLQGIKAKFQEKYQKSLSDMVRSDTSGDFRKLLVALLH

SEQ ID NO: 18 - protein, 265 aa, Homo Sapiens
  MISC_FEATURE (1)..(265): Accession No Q14002
MGSPSACPYRVCIPWQGLLLTASLLTFWNLPNSAQTNIDVVPFNVAEGKEVLLVVHNESQNLYGYNWYKGERVHANYRIIGYVKNISQENAPGPAHNGRETIYPNGTLLIQNVTHNDAGFYTLHVIKENLVNEEVTRQFYVFSEPPKPSITSNNFNPVENKDIVVLTCQPETQNTTYLWWVNNQSLLVSPRLLLSTDNRTLVLLSATKNDIGPYECEIQNPVGASRSDPVTLNVRYESVQASSPDLSAGTAVSIMIGVLAGMALI

SEQ ID NO: 19 - protein, 765 aa, Homo Sapiens
  DOMAIN (1)..(765): Extracellular domain of Q12864
QEGKFSGPLKPMTFSIYEGQEPSQIIFQFKANPPAVTFELTGETDNIFVIEREGLLYYNRALDRETRSTHNLQVAALDANGIIVEGPVPITIKVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGM

SEQ ID NO: 20 - protein, 108 aa, Homo Sapiens
  MISC_FEATURE (1)..(108): Commercially available recombinant protein of Q12864
EGKFSGPLKPMTFSIYEGQEPSQIIFQFKANPPAVTFELTGETDNIFVIEREGLLYYNRALDRETRSTHNLQVAALDANGIIVEGPVPITIEVKDINDNRPTFLQSKY

SEQ ID NO: 21 - protein, 214 aa, Homo Sapiens
  DOMAIN (1)..(214): Extracellualr domain of Q99795
ISVETPQDVLRASQGKSVTLPCTYHTSTSSREGLIQWDKLLLTHTERVVIWPFSNKNYIHGELYKNRVSISNNAEQSDASITIDQLTMADNGTYECSVSLMSDLEGNTKSRVRLLVLVPPSKPECGIEGETIIGNNIQLTCQSKEGSPTPQYSWKRYNILNQEQPLAQPASGQPVSLKNISTDTSGYYICTSSNEEGTQFCNITVAVRSPSMNV

SEQ ID NO: 22 - protein, 525 aa, Homo Sapiens
  DOMAIN (1)..(525): Extracellular domain of P29323
VEETLMDSTTATAELGWMVHPPSGWEEVSGYDENMNTIRTYQVCNVFESSQNNWLRTKFIRRRGAHRIHVEMKFSVRDCSSIPSVPGSCKETFNLYYYEADFDSATKTFPNWMENPWVKVDTIAADESFSQVDLGGRVMKINTEVRSFGPVSRSGFYLAFQDYGGCMSLIAVRVFYRKCPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDLVYNIICKSCGSGRGACTRCGDNVQYAPRQLGLTEPRIYISDLLAHTQYTFEIQAVNGVTDQSPFSPQFASVNITTNQAAPSAVSIMHQVSRTVDSITLSWSQPDQPNGVILDYELQYYEKELSEYNATAIKSPTNTVTVQGLKAGAIYVFQVRARTVAGYGRYSGKMYFQTMTEAEYQTSIQEKLPL

SEQ ID NO: 23 - protein, 100 aa, Homo Sapiens
  MISC_FEATURE (1)..(100): Commercially available recombinant protein of P29323
CIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIP

SEQ ID NO: 24 - protein, 487 aa, Homo Sapiens
  MISC_FEATURE (1)..(487): Commercially available recombinant protein of P29323
GFERADSEYTDKLQHYTSGHMTPGMKIYIDPFTYEDPNEAVREFAKEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPNSLKAMAPLSSGINLPLLDRTIPDYTSFNTVDEWLEAIKMGQYKESFANAGFTSFDVVSQMMMEDILRVGVTLAGHQKKILNSIQVMRAQMNQIQSVEGQPLARRPRATGRTKRCQPRDVTKKTCNSNDGKKKGMGKKKTDPGRGREIQGIFFKEDSHKESNDCSCGG

SEQ ID NO: 25 - protein, 779 aa, Homo Sapiens
  DOMAIN (1)..(779): Extracellular domain of Q9Y5Y6
WHLQYRDVRVQKVFNGYMRITNENFVDAYENSNSTEFVSLASKVKDALKLLYSGVPFLGPYHKESAVTAFSEGSVIAYYWSEFSIPQHLVEEAERVMAEERVVMLPPRARSLKSFVVTSVVAFPTDSKTVQRTQDNSCSFGLHARGVELMRFTTPGFPDSPYPAHARCQWALRGDADSVLSLTFRSFDLASCDERGSDLVTVYNTLSPMEPHALVQLCGTYPPSYNLTFHSSQNVLLITLITNTERRHPGFEATFFQLPRMSSCGGRLRKAQGTFNSPYYPGHYPPNIDCTWNIEVPNNQHVKVRFKFFYLLEPGVPAGTCPKDYVEINGEKYCGERSQFVVTSNSNKITVRFHSDQSYTDTGFLAEYLSYDSSDPCPGQFTCRTGRCIRKELRCDGWADCTDHSDELNCSCDAGHQFTCKNKFCKPLFWVCDSVNDCGDNSDEQGCSCPAQTFRCSNGKCLSKSQQCNGKDDCGDGSDEASCPKVNVVTCTKHTYRCLNGLCLSKGNPECDGKEDCSDGSDEKDCDCGLRSFTRQARVVGGTDADEGEWPWQVSLHALGQGHICGASLISPNWLVSAAHCYIDDRGFRYSDPTQWTAFLGLHDQSQRSAPGVQERRLKRIISHPFFNDFTFDYDIALLELEKPAEYSSMVRPICLPDASHVFPAGKAIWVTGWGHTQYGGTGALILQKGEIRVINQTTCENLLPQQITPRMMCVGFLSGGVDSCQGDSGGPLSSVEADGRIFQAGVVSWGDGCAQRNKPGVYTRLPLFRDWIKENTGV

SEQ ID NO: 26 - protein, 103 aa, Homo Sapiens
  MISC_FEATURE (1)..(103): Commercially available recombinant protein of Q9Y5Y6
PPSYNLTFHSSQNVLLITLITNTERRHPGFEATFFQLPRMSSCGGRLRKAQGTFNSPYYPGHYPPNIDCTWNIEVPNNQHVKVRFKFFYLLEPGVPAGTCPKD

SEQ ID NO: 27 - protein, 123 aa, Homo Sapiens
  DOMAIN (1)..(123): Extracellular domain of P18433
NNATTVAPSVGITRLINSSTAEPVKEEAKTSNPTSSLTSLSVAPTFSPNITLGPTYLTTVNSSDSDNGTTRTASTNSIGITISPNGTWLPDNQFTDARTEPWEGNSSTAATTPETFPPSDETP

SEQ ID NO: 28 - protein, 99 aa, Homo Sapiens
  MISC_FEATURE (1)..(99): Commercially available recombinant protein of Q6PIM3
SLKVKGGASELQEDESFTLRGPPGAAPSATQITVVLPHSSCELLYLGTESGNVFVVQLPAFRALEDRTISSDAVLQRLPEEARHRRVFEMVEALQEHPR

SEQ ID NO: 29 - protein, 663 aa, Homo Sapiens
  DOMAIN (1)..(663): Extracellular domain of Q9UN66
AEPRSYSVVEETEGSSFVTNLAKDLGLEQREFSRRGVRVVSRGNKLHLQLNQETADLLLNEKLDREDLCGHTEPCVLRFQVLLESPFEFFQAELQVIDINDHSPVFLDKQMLVKVSESSPPGTAFPLKNAEDLDIGQNNIENYIISPNSYFRVLTRKRSDGRKYPELVLDNALDREEEAELRLTLTALDGGSPPRSGTAQVYIEVVDVNDNAPEFQQPFYRVQISEDSPISFLVVKVSATDVDTGVNGEISYSLFQASDEISKTFKVDFLTGEIRLKKQLDFEKFQSYEVNIEARDAGGFSGKCTVLIQVIDVNDHAPEVTMSAFTSPIPENAPETVVALFSVSDLDSGENGKISCSIQEDLPFLLKSSVGNFYTLLTETPLDRESRAEYNVTITVTDLGTPRLTTHLNMTVLVSDVNDNAPAFTQTSYTLFVRENNSPALHIGSVSATDRDSGTNAQVTYSLLPPQDPHLPLASLVSINTDNGHLFALRSLDYEALQAFEFRVGASDRGSPALSSEALVRVLVLDANDNSPFVLYPLQNGSAPCTELVPRAAEPGYLVTKVVAVDGDSGQNAWLSYQLLKATEPGLFGVWAHNGEVRTARLLSERDAAKQRLVVLVKDNGEPPCSATATLHLLLVDGFSQPYLPLPEAAPAQGQADSLTVYL

SEQ ID NO: 30 - protein, 242 aa, Homo Sapiens
  DOMAIN (1)..(242): Extracellular domain of P16422
QEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLK

SEQ ID NO: 31 - protein, 90 aa, Homo Sapiens
  MISC_FEATURE (1)..(90): Commercially available recombinant protein of O95994
RDTTVKPGAKKDTKDSRPKLPQTLSRGWGDQLIWTQTYEEALYKSKTSNKPLMIIHHLDECPHSQALKKVFAENKEIQKLAEQFVLLNLV

SEQ ID NO: 32 - protein, 620 aa, Homo Sapiens
  DOMAIN (1)..(620): Extracellular domain of P01833
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSR

SEQ ID NO: 33 - protein, 110 aa, Homo Sapiens
  MISC_FEATURE (1)..(110): Commercially available recombinant protein of P01833
PIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQG

SEQ ID NO: 34 - protein, 11 aa, Homo Sapiens
AAGSRDVSLAK

SEQ ID NO: 35 - protein, 8 aa, Homo Sapiens
ADAAPDEK

SEQ ID NO: 36 - protein, 11 aa, Homo Sapiens
ADEGWYWCGVK

SEQ ID NO: 37 - protein, 12 aa, Homo Sapiens
AENPEPLVFGVK

SEQ ID NO: 38 - protein, 9 aa, Homo Sapiens
AEVDLQGIK

SEQ ID NO: 39 - protein, 16 aa, Homo Sapiens
AEYNVTITVTDLGTPR

SEQ ID NO: 40 - protein, 10 aa, Homo Sapiens
AFVNCDENSR

SEQ ID NO: 41 - protein, 7 aa, Homo Sapiens
ALLSDER

SEQ ID NO: 42 - protein, 26 aa, Homo Sapiens
ANLTNFPENGTFVVNIAQLSQDDSGR

SEQ ID NO: 43 - protein, 7 aa, Homo Sapiens
APAFEGR

SEQ ID NO: 44 - protein, 10 aa, Homo Sapiens
APEFSMQGLK

SEQ ID NO: 45 - protein, 6 aa, Homo Sapiens
APYEWK

SEQ ID NO: 46 - protein, 6 aa, Homo Sapiens
AQIHMR

SEQ ID NO: 47 - protein, 6 aa, Homo Sapiens
AQYEGR

SEQ ID NO: 48 - protein, 11 aa, Homo Sapiens
ASSPQGFDVDR

SEQ ID NO: 49 - protein, 15 aa, Homo Sapiens
ASSPQGFDVDRDAKK

SEQ ID NO: 50 - protein, 16 aa, Homo Sapiens
ASVDSGSSEEQGGSSR

SEQ ID NO: 51 - protein, 14 aa, Homo Sapiens
ATFAFSPEEQQAQR

SEQ ID NO: 52 - protein, 12 aa, Homo Sapiens
ATFQAYQILIGK

SEQ ID NO: 53 - protein, 9 aa, Homo Sapiens
AVINSAGYK

SEQ ID NO: 54 - protein, 7 aa, Homo Sapiens
AYLTLVR

SEQ ID NO: 55 - protein, 7 aa, Homo Sapiens
AYPQYYR

SEQ ID NO: 56 - protein, 12 aa, Homo Sapiens
CAQDCEDYFAER

SEQ ID NO: 57 - protein, 8 aa, Homo Sapiens
CGLGINSR

SEQ ID NO: 58 - protein, 12 aa, Homo Sapiens
CPLLVDSEGWVK

SEQ ID NO: 59 - protein, 15 aa, Homo Sapiens
CPTQFPLILWHPYAR

SEQ ID NO: 60 - protein, 16 aa, Homo Sapiens
CSVPEGPFPGHLVDVR

SEQ ID NO: 61 - protein, 16 aa, Homo Sapiens
DAGFYWCLTNGDTLWR

SEQ ID NO: 62 - protein, 10 aa, Homo Sapiens
DAYVFYAVAK

SEQ ID NO: 63 - protein, 11 aa, Homo Sapiens
DEDEDIQSILR

SEQ ID NO: 64 - protein, 13 aa, Homo Sapiens
DEENTANSFLNYR

SEQ ID NO: 65 - protein, 16 aa, Homo Sapiens
DEYGKPLSYPLEIHVK

SEQ ID NO: 66 - protein, 12 aa, Homo Sapiens
DGSFSVVITGLR

SEQ ID NO: 67 - protein, 16 aa, Homo Sapiens
DIEEAIEEETSGDLQK

SEQ ID NO: 68 - protein, 13 aa, Homo Sapiens
DINDNRPTFLQSK

SEQ ID NO: 69 - protein, 9 aa, Homo Sapiens
DLYDAGEGR

SEQ ID NO: 70 - protein, 15 aa, Homo Sapiens
DNVESAQASEVKPLR

SEQ ID NO: 71 - protein, 7 aa, Homo Sapiens
DQADGSR

SEQ ID NO: 72 - protein, 7 aa, Homo Sapiens
DRNHRPK

SEQ ID NO: 73 - protein, 9 aa, Homo Sapiens
DTEITCSER

SEQ ID NO: 74 - protein, 14 aa, Homo Sapiens
DVSLAKADAAPDEK

SEQ ID NO: 75 - protein, 7 aa, Homo Sapiens
DYEILFK

SEQ ID NO: 76 - protein, 11 aa, Homo Sapiens
EAYEEPPEQLR

SEQ ID NO: 77 - protein, 14 aa, Homo Sapiens
EEFVATTESTTETK

SEQ ID NO: 78 - protein, 9 aa, Homo Sapiens
EELCKSIQR

SEQ ID NO: 79 - protein, 9 aa, Homo Sapiens
EGHQSEGLR

SEQ ID NO: 80 - protein, 8 aa, Homo Sapiens
EGLIQWDK

SEQ ID NO: 81 - protein, 8 aa, Homo Sapiens
EGLLYYNR

SEQ ID NO: 82 - protein, 11 aa, Homo Sapiens
EGSPTPQYSWK

SEQ ID NO: 83 - protein, 7 aa, Homo Sapiens
EKPYDSK

SEQ ID NO: 84 - protein, 7 aa, Homo Sapiens
ELEIPPR

SEQ ID NO: 85 - protein, 7 aa, Homo Sapiens
EMGEMHR

SEQ ID NO: 86 - protein, 9 aa, Homo Sapiens
EREEEDDYR

SEQ ID NO: 87 - protein, 14 aa, Homo Sapiens
EREEEDDYRQEEQR

SEQ ID NO: 88 - protein, 17 aa, Homo Sapiens
EVTDMNLNVINEGGIDK

SEQ ID NO: 89 - protein, 12 aa, Homo Sapiens
FDTHEYRNESRR

SEQ ID NO: 90 - protein, 7 aa, Homo Sapiens
FEEVLSK

SEQ ID NO: 91 - protein, 12 aa, Homo Sapiens
FFNVLTTNTDGK

SEQ ID NO: 92 - protein, 10 aa, Homo Sapiens
FGQIVNTLDK

SEQ ID NO: 93 - protein, 16 aa, Homo Sapiens
FIGVEAGGTLELHGAR

SEQ ID NO: 94 - protein, 13 aa, Homo Sapiens
FIMLNLMHETTDK

SEQ ID NO: 95 - protein, 10 aa, Homo Sapiens
FLRPGHDPVR

SEQ ID NO: 96 - protein, 7 aa, Homo Sapiens
FQEKYQK

SEQ ID NO: 97 - protein, 14 aa, Homo Sapiens
FQEKYQKSLSDMVR

SEQ ID NO: 98 - protein, 11 aa, Homo Sapiens
FQELIFEDFAR

SEQ ID NO: 99 - protein, 13 aa, Homo Sapiens
FRPHQDANPEKPR

SEQ ID NO: 100 - protein, 6 aa, Homo Sapiens
FSSYEK

SEQ ID NO: 101 - protein, 16 aa, Homo Sapiens
FTTPGFPDSPYPAHAR

SEQ ID NO: 102 - protein, 11 aa, Homo Sapiens
GAGTDEETLIR

SEQ ID NO: 103 - protein, 12 aa, Homo Sapiens
GDADSVLSLTFR

SEQ ID NO: 104 - protein, 8 aa, Homo Sapiens
GDTRGWLK

SEQ ID NO: 105 - protein, 9 aa, Homo Sapiens
GDTSGNLKK

SEQ ID NO: 106 - protein, 8 aa, Homo Sapiens
GESLFHSK

SEQ ID NO: 107 - protein, 15 aa, Homo Sapiens
GGASELQEDESFTLR

SEQ ID NO: 108 - protein, 16 aa, Homo Sapiens
GGCITLISSEGYVSSK

SEQ ID NO: 109 - protein, 15 aa, Homo Sapiens
GHSPAFLQPQNGNSR

SEQ ID NO: 110 - protein, 16 aa, Homo Sapiens
GMGTNEAAIIEILSGR

SEQ ID NO: 111 - protein, 18 aa, Homo Sapiens
GSVTFHCALGPEVANVAK

SEQ ID NO: 112 - protein, 18 aa, Homo Sapiens
GWGDQLIWTQTYEEALYK

SEQ ID NO: 113 - protein, 12 aa, Homo Sapiens
GYTIHWNGPAPR

SEQ ID NO: 114 - protein, 16 aa, Homo Sapiens
HEIEGTGLPQAQLLWR

SEQ ID NO: 115 - protein, 11 aa, Homo Sapiens
HLSPDGQYVPR

SEQ ID NO: 116 - protein, 5 aa, Homo Sapiens
HNLYR

SEQ ID NO: 117 - protein, 13 aa, Homo Sapiens
HPGFEATFFQLPR

SEQ ID NO: 118 - protein, 7 aa, Homo Sapiens
HTEFEER

SEQ ID NO: 119 - protein, 16 aa, Homo Sapiens
IDHVTGEIFSVAPLDR

SEQ ID NO: 120 - protein, 15 aa, Homo Sapiens
IEEYEPVHSLEELQR

SEQ ID NO: 121 - protein, 11 aa, Homo Sapiens
IEFISTMEGYK

SEQ ID NO: 122 - protein, 16 aa, Homo Sapiens
IFQAGVVSWGDGCAQR

SEQ ID NO: 123 - protein, 9 aa, Homo Sapiens
IIEGEPNLK

SEQ ID NO: 124 - protein, 8 aa, Homo Sapiens
ILLNPQDK

SEQ ID NO: 125 - protein, 10 aa, Homo Sapiens
ILVSLLQANR

SEQ ID NO: 126 - protein, 11 aa, Homo Sapiens
IMFVDPSLTVR

SEQ ID NO: 127 - protein, 14 aa, Homo Sapiens
IPIRWTAPEAIQYR

SEQ ID NO: 128 - protein, 15 aa, Homo Sapiens
IVFSGNLFQHQEDSK

SEQ ID NO: 129 - protein, 8 aa, Homo Sapiens
KELEIPPR

SEQ ID NO: 130 - protein, 13 aa, Homo Sapiens
KFCIQQVGDMTNR

SEQ ID NO: 131 - protein, 13 aa, Homo Sapiens
KFFNVLTTNTDGK

SEQ ID NO: 132 - protein, 6 aa, Homo Sapiens
KHNLYR

SEQ ID NO: 133 - protein, 6 aa, Homo Sapiens
KKRMAK

SEQ ID NO: 134 - protein, 20 aa, Homo Sapiens
KNADLQVLKPEPELVYEDLR

SEQ ID NO: 135 - protein, 7 aa, Homo Sapiens
KNNHHFK

SEQ ID NO: 136 - protein, 7 aa, Homo Sapiens
KVFAENK

SEQ ID NO: 137 - protein, 10 aa, Homo Sapiens
KYDYDSSSVR

SEQ ID NO: 138 - protein, 11 aa, Homo Sapiens
KYDYDSSSVRK

SEQ ID NO: 139 - protein, 12 aa, Homo Sapiens
KYDYDSSSVRKR

SEQ ID NO: 140 - protein, 5 aa, Homo Sapiens
KYWCR

SEQ ID NO: 141 - protein, 9 aa, Homo Sapiens
LAAKCLVMK

SEQ ID NO: 142 - protein, 19 aa, Homo Sapiens
LDIQGTGQLLFSVVINQLR

SEQ ID NO: 143 - protein, 6 aa, Homo Sapiens
LFAEEK

SEQ ID NO: 144 - protein, 11 aa, Homo Sapiens
LFDRSLESDVK

SEQ ID NO: 145 - protein, 7 aa, Homo Sapiens
LGEYMEK

SEQ ID NO: 146 - protein, 8 aa, Homo Sapiens
LLLTHTER

SEQ ID NO: 147 - protein, 7 aa, Homo Sapiens
LNLWISR

SEQ ID NO: 148 - protein, 7 aa, Homo Sapiens
LPDNTVK

SEQ ID NO: 149 - protein, 7 aa, Homo Sapiens
LPQTLSR

SEQ ID NO: 150 - protein, 18 aa, Homo Sapiens
LPSVEEAEVPKPLPPASK

SEQ ID NO: 151 - protein, 12 aa, Homo Sapiens
LQEDGLSVWFQR

SEQ ID NO: 152 - protein, 22 aa, Homo Sapiens
LSLLEEPGNGTFTVILNQLTSR

SEQ ID NO: 153 - protein, 17 aa, Homo Sapiens
LYAYEPADTALLLDNMK

SEQ ID NO: 154 - protein, 7 aa, Homo Sapiens
LYTYEPR

SEQ ID NO: 155 - protein, 7 aa, Homo Sapiens
MDNYLLR

SEQ ID NO: 156 - protein, 12 aa, Homo Sapiens
MESLRLDGLQQR

SEQ ID NO: 157 - protein, 7 aa, Homo Sapiens
MGWMGEK

SEQ ID NO: 158 - protein, 9 aa, Homo Sapiens
MIRNPNSLK

SEQ ID NO: 159 - protein, 15 aa, Homo Sapiens
MPAMLTGLCQGCGTR

SEQ ID NO: 160 - protein, 13 aa, Homo Sapiens
NLDGISHAPNAVK

SEQ ID NO: 161 - protein, 11 aa, Homo Sapiens
NLSPDGQYVPR

SEQ ID NO: 162 - protein, 12 aa, Homo Sapiens
NLSSTTDDEAPR

SEQ ID NO: 163 - protein, 6 aa, Homo Sapiens
NNHHFK

SEQ ID NO: 164 - protein, 8 aa, Homo Sapiens
NSWQLTPR

SEQ ID NO: 165 - protein, 9 aa, Homo Sapiens
NYIHGELYK

SEQ ID NO: 166 - protein, 10 aa, Homo Sapiens
QAGSHSNSFR

SEQ ID NO: 167 - protein, 14 aa, Homo Sapiens
QETDYVLNNGFNPR

SEQ ID NO: 168 - protein, 18 aa, Homo Sapiens
QGHFYGETAAVYVAVEER

SEQ ID NO: 169 - protein, 19 aa, Homo Sapiens
QGHFYGETAAVYVAVEERK

SEQ ID NO: 170 - protein, 8 aa, Homo Sapiens
QLGLTEPR

SEQ ID NO: 171 - protein, 15 aa, Homo Sapiens
QPGLVMERALLSDER

SEQ ID NO: 172 - protein, 16 aa, Homo Sapiens
QSSGENCDVVVNTLGK

SEQ ID NO: 173 - protein, 17 aa, Homo Sapiens
QSSGENCDVVVNTLGKR

SEQ ID NO: 174 - protein, 13 aa, Homo Sapiens
QVVEAAQAPIQER

SEQ ID NO: 175 - protein, 8 aa, Homo Sapiens
RAPAFEGR

SEQ ID NO: 176 - protein, 24 aa, Homo Sapiens
RATASEQPLAQEPPASGGSPATTK

SEQ ID NO: 177 - protein, 14 aa, Homo Sapiens
REDVSGIASCVFTK

SEQ ID NO: 178 - protein, 16 aa, Homo Sapiens
RKNLDLAAPTAEEAQR

SEQ ID NO: 179 - protein, 16 aa, Homo Sapiens
RPELEEIFHQYSGEDR

SEQ ID NO: 180 - protein, 8 aa, Homo Sapiens
RPPQTLSR

SEQ ID NO: 181 - protein, 6 aa, Homo Sapiens
RSDYAK

SEQ ID NO: 182 - protein, 8 aa, Homo Sapiens
RSESVKSR

SEQ ID NO: 183 - protein, 11 aa, Homo Sapiens
SAEDSFTGFVR

SEQ ID NO: 184 - protein, 13 aa, Homo Sapiens
SDEGESMPTFGKK

SEQ ID NO: 185 - protein, 9 aa, Homo Sapiens
SDPVTLNVR

SEQ ID NO: 186 - protein, 8 aa, Homo Sapiens
SDTSGDFR

SEQ ID NO: 187 - protein, 9 aa, Homo Sapiens
SELSGNFEK

SEQ ID NO: 188 - protein, 7 aa, Homo Sapiens
SESEEEK

SEQ ID NO: 189 - protein, 15 aa, Homo Sapiens
SFVVTSVVAFPTDSK

SEQ ID NO: 190 - protein, 15 aa, Homo Sapiens
SINGILFPGGSVDLR

SEQ ID NO: 191 - protein, 16 aa, Homo Sapiens
SINGILFPGGSVDLRR

SEQ ID NO: 192 - protein, 12 aa, Homo Sapiens
SLAPGMALGSGR

SEQ ID NO: 193 - protein, 7 aa, Homo Sapiens
SLESDVK

SEQ ID NO: 194 - protein, 7 aa, Homo Sapiens
SLSDMVR

SEQ ID NO: 195 - protein, 15 aa, Homo Sapiens
SVTLPCTYHTSTSSR

SEQ ID NO: 196 - protein, 14 aa, Homo Sapiens
TAFYLAEFFVNEAR

SEQ ID NO: 197 - protein, 15 aa, Homo Sapiens
TALALLDRPSEYAAR

SEQ ID NO: 198 - protein, 12 aa, Homo Sapiens
TDISMSDFENSR

SEQ ID NO: 199 - protein, 10 aa, Homo Sapiens
TDMDQIITSK

SEQ ID NO: 200 - protein, 14 aa, Homo Sapiens
TEDVEPQSVPLLAR

SEQ ID NO: 201 - protein, 7 aa, Homo Sapiens
TENAQKR

SEQ ID NO: 202 - protein, 8 aa, Homo Sapiens
TGAISLTR

SEQ ID NO: 203 - protein, 10 aa, Homo Sapiens
TLEDEEEQER

SEQ ID NO: 204 - protein, 16 aa, Homo Sapiens
TLNSSGLPFGSYTFEK

SEQ ID NO: 205 - protein, 9 aa, Homo Sapiens
TLVLLSATK

SEQ ID NO: 206 - protein, 10 aa, Homo Sapiens
TLYFADTYLK

SEQ ID NO: 207 - protein, 16 aa, Homo Sapiens
TQNDVDIADVAYYFEK

SEQ ID NO: 208 - protein, 19 aa, Homo Sapiens
TQNDVDIADVAYYFEKDVK

SEQ ID NO: 209 - protein, 11 aa, Homo Sapiens
TVAGYGRYSGK

SEQ ID NO: 210 - protein, 9 aa, Homo Sapiens
TVTINCPFK

SEQ ID NO: 211 - protein, 11 aa, Homo Sapiens
VEGPAFTDAIR

SEQ ID NO: 212 - protein, 15 aa, Homo Sapiens
VFAQNEEIQEMAQNK

SEQ ID NO: 213 - protein, 13 aa, Homo Sapiens
VFEMVEALQEHPR

SEQ ID NO: 214 - protein, 7 aa, Homo Sapiens
VLDSGFR

SEQ ID NO: 215 - protein, 12 aa, Homo Sapiens
VLDSGFREIENK

SEQ ID NO: 216 - protein, 8 aa, Homo Sapiens
VPCHFPCK

SEQ ID NO: 217 - protein, 7 aa, Homo Sapiens
VPPAERR

SEQ ID NO: 218 - protein, 13 aa, Homo Sapiens
VQQVQPAMQAVIR

SEQ ID NO: 219 - protein, 8 aa, Homo Sapiens
VSDFGLSR

SEQ ID NO: 220 - protein, 10 aa, Homo Sapiens
VSEDVALGTK

SEQ ID NO: 221 - protein, 9 aa, Homo Sapiens
VSVAHFGSR

SEQ ID NO: 222 - protein, 16 aa, Homo Sapiens
VTVDAISVETPQDVLR

SEQ ID NO: 223 - protein, 7 aa, Homo Sapiens
VVMLPPR

SEQ ID NO: 224 - protein, 8 aa, Homo Sapiens
VYTVDLGR

SEQ ID NO: 225 - protein, 14 aa, Homo Sapiens
WGTDELAFNEVLAK

SEQ ID NO: 226 - protein, 15 aa, Homo Sapiens
WGTDELAFNEVLAKR

SEQ ID NO: 227 - protein, 13 aa, Homo Sapiens
WNDPGAQYSLVDK

SEQ ID NO: 228 - protein, 6 aa, Homo Sapiens
WSLSVK

SEQ ID NO: 229 - protein, 10 aa, Homo Sapiens
WTAPEAIQYR

SEQ ID NO: 230 - protein, 9 aa, Homo Sapiens
YDYDSSSVR

SEQ ID NO: 231 - protein, 10 aa, Homo Sapiens
YDYDSSSVRK

SEQ ID NO: 232 - protein, 11 aa, Homo Sapiens
YDYDSSSVRKR

SEQ ID NO: 233 - protein, 7 aa, Homo Sapiens
YEKAEIK

SEQ ID NO: 234 - protein, 15 aa, Homo Sapiens
YGQGFYLISPSEFER

SEQ ID NO: 235 - protein, 15 aa, Homo Sapiens
YGQGFYLLSPSEFER

SEQ ID NO: 236 - protein, 10 aa, Homo Sapiens
YKCGLGINSR

SEQ ID NO: 237 - protein, 10 aa, Homo Sapiens
YLADMNYVHR

SEQ ID NO: 238 - protein, 32 aa, Homo Sapiens
YLCGAHSDGQLQEGSPIQAWQLFVNEESTIPR

SEQ ID NO: 239 - protein, 8 aa, Homo Sapiens
YLESAGAR

SEQ ID NO: 240 - protein, 22 aa, Homo Sapiens
YNILNQEQPLAQPASGQPVSLK

SEQ ID NO: 241 - protein, 8 aa, Homo Sapiens
YPPLPVDK

SEQ ID NO: 242 - protein, 12 aa, Homo Sapiens
YPVYGVQWHPEK

SEQ ID NO: 243 - protein, 18 aa, Homo Sapiens
YPVYGVQWHPEKAPYEWK

SEQ ID NO: 244 - protein, 12 aa, Homo Sapiens
YWCLWEGAQNGR

SEQ ID NO: 245 - protein, 9 aa, Homo Sapiens
YYIAASYVK

(19) United States
(12) Patent Application Publication
(10) Pub. No.: US 2009/0169575 A1
(43) Pub. Date: Jul. 2, 2009

(54) PROTEINS

(76) Inventor: Christian Rohlff, Abingdon (GB)

Correspondence Address: KLAUBER & JACKSON, 411 HACKENSACK AVENUE, HACKENSACK, NJ, 07601, US

(21) Appl. No.: 12/329,500

(22) Filed: Dec. 5, 2008

Related U.S. Application Data
(63) Continuation of application No. PCT/EP2007/055537, filed on Jun. 5, 2007.
(60) Provisional application No. 60/811,681, filed on Jun. 7, 2006.

(30) Foreign Application Priority Data
Jun. 6, 2006 (GB) GB0611116.5

(51) Int. Cl. A61K 39/00; G01N 33/53; C07K 16/18; G01N 33/68; G01N 33/574

(52) U.S. Cl. 424/185.1; 435/7.1; 530/387.9; 436/86; 435/7.23

(57) ABSTRACT

The present invention provides methods and compositions for screening, diagnosis and prognosis of colorectal cancer, for monitoring the effectiveness of colorectal cancer treatment, and for drug development.

### RELATED APPLICATIONS

The present application is a Continuation of co-pending PCT Application No. PCT/EP2007/055537 filed Jun. 5, 2007, which in turn, claims priority from G.B. Application No. 0611116.5 filed Jun. 6, 2006 and U.S. Provisional Application Ser. No. 60/811,681 filed Jun. 7, 2006. Applicants claim the benefits of 35 U.S.C. § 120 as to the PCT application and priority under 35 U.S.C. § 119 as to the said G.B. and U.S. Provisional applications, and the entire disclosures of all applications are incorporated herein by reference in their entireties.

### INTRODUCTION

The present invention relates to the identification of marker proteins not previously reported for human colorectal cancer which have utility as diagnostic and prognostic markers for colorectal cancer and colorectal cancer metastases. These proteins may also form biological targets against which therapeutic antibodies (or other affinity reagents such as Affibodies, Nanobodies or Unibodies) or other pharmaceutical agents can be made.

### BACKGROUND OF THE INVENTION

### Colorectal Cancer

Colorectal cancer (CRC) is one of the leading causes of cancer-related morbidity and mortality, responsible for an estimated half a million deaths per year, mostly in Western, well developed countries. In these territories, CRC is the third most common malignancy (estimated number of new cases per annum in USA and EU is approximately 350,000 per year). Estimated healthcare costs related to treatment for colorectal cancer in the United States are more than $8 billion.

### Colorectal Cancer Diagnosis:

Today, the fecal occult blood test and colonoscopy, a highly invasive procedure, are the most frequently used screening and diagnostic methods for colorectal cancer. Other diagnostic tools include Flexible Sigmoidoscopy (allowing the observation of only about half of the colon) and Double Contrast Barium Enema (DCBE, to obtain X-ray images).

### Colorectal Cancer Staging:

CRC has four distinct stages: patients with stage I disease have a five-year survival rate of >90%, while those with metastatic stage IV disease have a <5% survival rate according to the US National Institutes of Health (NIH).

### Colorectal Cancer Treatment:

Once CRC has been diagnosed, the correct treatment needs to be selected. Surgery is usually the main treatment for colorectal cancer, although radiation and chemotherapy will often be given before surgery. Possible side effects of surgery include bleeding from the surgery, blood clots in the legs, and damage to nearby organs during the operation.

Currently, 60 percent of colorectal cancer patients receive chemotherapy to treat their disease; however, this form of treatment only benefits a few percent of the population, while carrying with it high risks of toxicity, thus demonstrating a need to better define the patient selection criteria.

Colorectal cancer has a 30 to 40 percent recurrence rate within an average of 18 months after primary diagnosis. As with all cancers, the earlier it is detected the more likely it can be cured, especially as pathologists have recognised that the majority of CRC tumours develop in a series of well-defined stages from benign adenomas.

Colon Cancer Survival by Stage

| Stage | Survival Rate |
|---|---|
| I | 93% |
| IIA | 85% |
| IIB | 72% |
| IIIA | 83% |
| IIIB | 64% |
| IIIC | 44% |
| IV | 8% |

### Therapeutic Challenges

The major challenges in colorectal cancer treatment are to improve early detection rates, to find new non-invasive markers that can be used to follow disease progression and identify relapse, and to find improved and less toxic therapies, especially for more advanced disease where 5 year survival is still very poor. There is a great need to identify targets which are more specific to the cancer cells e.g. ones which are expressed on the surface of the tumour cells so that they can be attacked by promising new approaches like immunotherapeutics and targeted toxins.

### SUMMARY OF THE INVENTION

The present invention provides methods and compositions for screening, diagnosis, prognosis and therapy of colorectal cancer, for colorectal cancer patients' stratification, for monitoring the effectiveness of colorectal cancer treatment, and for drug development for treatment of colorectal cancer.

We have used mass spectrometry to identify peptides generated by gel electrophoresis and tryptic digest of membrane proteins extracted from colorectal tissue samples. Peptide sequences were compared to existing protein and cDNA databases and the corresponding gene sequences identified. For these membrane proteins, soluble forms exist, e.g. in serum, some of which are reported herein, and others which are known in the art. Many of these have not been previously reported to originate from colorectal cell membranes and represent a new set of proteins of potential diagnostic and/or therapeutic value.

Thus, a first aspect of the invention provides methods for diagnosis of colorectal cancer that comprises analysing a sample of serum e.g. by two-dimensional electrophoresis to detect at least one Colorectal Cancer Marker Protein (CRCMP), e.g., one or more of the CRCMPs disclosed herein or any combination thereof. These methods are also suitable for screening, prognosis, monitoring the results of therapy, drug development and discovery of new targets for drug treatment.

In particular there is provided a method of diagnosing colorectal cancer in a subject, differentiating causes of colorectal cancer in a subject, guiding therapy in a subject suffering from colorectal cancer, assessing the risk of relapse in a subject suffering from colorectal cancer, or assigning a prognostic risk of one or more future clinical outcomes to a subject suffering from colorectal cancer, the method comprising:

(a) performing assays configured to detect a soluble polypeptide derived from a protein selected from the list consisting of proteins defined by SEQ ID Nos 1-18 as a marker in one or more samples obtained from said subject; and
(b) correlating the results of said assay(s) to the presence or absence of colorectal cancer in the subject, to a therapeutic regimen to be used in the subject, to a risk of relapse in the subject, or to the prognostic risk of one or more clinical outcomes for the subject suffering from colorectal cancer.

Suitably such a method involves determining that when the level of said detected marker is higher in the subject than a control level, said determination indicates the presence of colorectal cancer in the subject, indicates a greater risk of relapse in the subject, or indicates a worse prognosis for the subject. Suitably if the level of said detected marker reduced in response to therapy, this indicates that the subject is responding to therapy. In particular such a method is a method for diagnosing colorectal cancer in a subject.

Diagnosing cancer embraces diagnosing primary cancer and relapse.

Colorectal cancer includes metastatic colorectal cancer.

Suitably the method may comprise performing one or more additional assays configured to detect one or more additional markers in addition to the soluble polypeptide derived from a protein selected from the list consisting of proteins defined by SEQ ID Nos 1-18 and wherein said correlating step comprises correlating the results of said assay(s) and the results of said additional assay(s) to the presence or absence of colorectal cancer in the subject, to a risk of relapse in the subject, or to the prognostic risk of one or more clinical outcomes for the subject suffering from colorectal cancer.

Suitably in methods according to the invention the subject is a human.

There is also provided a method for identifying the presence or absence of colorectal cancer cells in a biological sample obtained from a human subject, which comprises the step of identifying the presence or absence of one or more soluble polypeptides derived from a protein selected from the list consisting of proteins defined by SEQ ID Nos 1-18.

The presence of a soluble polypeptide may typically be determined qualitatively or quantitatively (eg quantitatively) for example by a method involving imaging technology (eg use of a labeled affinity reagent such as an antibody or an Affibody) as described herein.

There is also provided a method of detecting, diagnosing colorectal cancer in a subject, differentiating causes of colorectal cancer in a subject, guiding therapy in a subject suffering from colorectal cancer, assessing the risk of relapse in a subject suffering from colorectal cancer, or assigning a prognostic risk of one or more future clinical outcomes to a subject suffering from colorectal cancer, the method comprising:

(a) bringing into contact with a sample to be tested from said subject one or more antibodies (or other affinity reagents such as Affibodies, Nanobodies or Unibodies) capable of specific binding to a soluble polypeptides derived from a protein selected from the list consisting of proteins defined by SEQ ID Nos 1-18; and
(b) thereby detecting the presence of one or more soluble polypeptides derived from a protein selected from the list consisting of proteins defined by SEQ ID Nos 1-18 in the sample.

In such a method the presence of one or more said soluble polypeptides may indicate the presence of colorectal cancer in the patient.

There is also provided a method for identifying the presence of colorectal cancer in a subject which comprises the step of carrying out a whole body scan of said subject to determine the localisation of colorectal cancer cells, particularly metastatic colorectal cancer cells, in order to determine presence or amount of one or more soluble polypeptides derived from a protein selected from the list consisting of proteins defined by SEQ ID Nos 1-18, wherein the presence or amount of one or more of said soluble polypeptides indicates the presence of colorectal cancer in the subject.

There is also provided a method for identifying the presence of colorectal cancer in a subject which comprises determining the localisation of colorectal cancer cells by reference to a whole body scan of said subject, which scan indicates the presence or amount of one or more soluble polypeptides derived from a protein selected from the list consisting of proteins defined by SEQ ID Nos 1-18, wherein the presence or amount of one or more of said soluble polypeptides indicates the presence of colorectal cancer in the subject.

There is also provided a method of detecting, diagnosing colorectal cancer in a subject, differentiating causes of colorectal cancer in a subject, guiding therapy in a subject suffering from colorectal cancer, assessing the risk of relapse in a subject suffering from colorectal cancer, or assigning a prognostic risk of one or more future clinical outcomes to a subject suffering from colorectal cancer, the method comprising:
(a) bringing into contact with a sample to be tested one or more soluble polypeptides derived from a protein selected from the list consisting of proteins defined by SEQ ID Nos 1-18, or one or more antigenic or immunogenic fragments thereof, and
(b) detecting the presence of antibodies (or other affinity reagents such as Affibodies, Nanobodies or Unibodies) in the subject capable of specific binding to one or more of said polypeptides, or antigenic or immunogenic fragments thereof.

A second aspect of the invention provides methods of treating colorectal cancer, comprising administering to a patient a therapeutically effective amount of a compound that modulates (e.g., upregulates or downregulates) or complements the expression or the biological activity (or both) of a CRCMP in patients having colorectal cancer, in order to (a) prevent the onset or development of colorectal cancer; (b) prevent the progression of colorectal cancer; or (c) ameliorate the symptoms of colorectal cancer.

A third aspect of the invention provides methods of screening for compounds that modulate (e.g., upregulate or downregulate) the expression or biological activity of a CRCMP.

A fourth aspect of the invention provides monoclonal and polyclonal antibodies or other affinity reagents such as Affibodies, Nanobodies or Unibodies capable of immunospecific binding to a CRCMP, e.g., a CRCMP disclosed herein.

Thus, in a fifth aspect, the present invention provides a method for screening for and/or diagnosis of colorectal cancer in a human subject, which method comprises the step of identifying the presence or absence of one or more of the CRCMPs as defined in Tables 1 and 2 herein, in a biological sample obtained from said human subject.

In a sixth aspect, the present invention provides a method for monitoring and/or assessing colorectal cancer treatment in a human subject, which comprises the step of identifying the presence or absence of one or more of the CRCMPs as defined in Tables 1 or 2 herein, in a biological sample obtained from said human subject.

In a seventh aspect, the present invention provides a method for identifying the presence or absence of metastatic colorectal cancer cells in a biological sample obtained from a human subject, which comprises the step of identifying the presence or absence of one or more of the CRCMPs as defined in Tables 1 or 2 herein.

In an eighth aspect, the present invention provides a method for monitoring and/or assessing colorectal cancer treatment in a human subject, which comprises the step of determining whether one or more of the CRCMPs as defined in Tables 1 or 2 herein is increased/decreased in a biological sample obtained from a patient.

The biological sample used can be from any source such as a serum sample or a tissue sample, e.g. colorectal tissue. For instance, when looking for evidence of metastatic colorectal cancer, one would look at major sites of colorectal cancer metastasis, e.g. the liver, the peritoneal cavity, the pelvis, the retroperitoneum and the lungs.

Preferably, the methods of the present invention are not based on looking for the presence or absence of all of the CRCMPs defined in Tables 1 and 2, but rather on “clusters” or groups thereof.

Other aspects of the present invention are set out below and in the claims herein.

### BRIEF DESCRIPTION OF THE FIGURES

FIGS. 1-5, 7 and 9 show Box plot data for CRCMP#19, CRCMP#6, CRCMP#22, CRCMP#10 and CRCMP#9 as described in Examples 3 and 4.

FIGS. 6 and 8 show ROC curve data for CRCMP#19 and CRCMP#9 respectively as described in Example 4.

FIGS. 10(a)-10(r) show the sequences of the CRCMPs and mass match and tandem peptide fragments etc which are discussed in the Examples.

### DETAILED DESCRIPTION OF THE INVENTION

The invention described in detail below provides methods and compositions for clinical screening, diagnosis and prognosis of colorectal cancer in a mammalian subject, for identifying patients most likely to respond to a particular therapeutic treatment, for monitoring the results of colorectal cancer therapy, for drug screening and drug development. The invention also encompasses the administration of therapeutic compositions to a mammalian subject to treat or prevent colorectal cancer. The mammalian subject may be a non-human mammal, but is preferably human, more preferably a human adult, i.e. a human subject at least 21 (more preferably at least 35, at least 50, at least 60, at least 70, or at least 80) years old. For clarity of disclosure, and not by way of limitation, the invention will be described with respect to the analysis of colon tissue and serum samples. However, as one skilled in the art will appreciate, the assays and techniques described below can be applied to other types of patient samples, including another body fluid (e.g. urine or saliva), a tissue sample from a patient at risk of having colorectal cancer (e.g. a biopsy such as a colon tissue biopsy) or homogenate thereof. The methods and compositions of the present invention are specially suited for screening, diagnosis and prognosis of a living subject, but may also be used for postmortem diagnosis in a subject, for example, to identify family members at risk of developing the same disease.

As used herein, colon tissue refers to the colon itself, as well as the tissue adjacent to and/or within the strata underlying the colon.

### Colorectal Cancer Marker Proteins (CRCMPs)

In one aspect of the invention, two-dimensional electrophoresis is used to analyze serum samples from a subject, preferably a living subject, in order to measure the expression of one or more Colorectal Cancer Marker Proteins (CRCMPs) for screening or diagnosis of colorectal cancer, to determine the prognosis of a colorectal cancer patient, or to monitor the effectiveness of colorectal cancer therapy.

As used herein, the term “Colorectal Cancer Marker Protein” (CRCMP) refers to a soluble polypeptide derived from a protein believed to be associated with colorectal cancer. 18 such proteins are recited in Tables 1 and 2 by reference to their accession numbers. Soluble polypeptides derived therefrom have been detected by 1 or 2D electrophoresis of colorectal cancer tissue sample as shown in Table 1 and 2. Table 2 recited those proteins that have been detected as features on a gel by 2D gel analysis and Table 1 recites those proteins that have been detected as features on a gel by 1D gel analysis.

In particular, some of the features in Tables 1 and 2 have entries in the SwissProt database (available online at http://www.expasy.org), which is an annotated database for proteins. For these entries, the SwissProt database contains information on the structure of the proteins, when known, and this includes a definition of the sequence making up soluble parts of the proteins. In addition, methods suitable for predicting soluble forms of membrane proteins include, but are not limited to, primary structure analysis to identify membrane spanning helices and extracellular domains, which is provided by a number of bioinformatics tools, such as the Dense Alignment Surface method, the HMMTOP method, the TMpred method, the TopPred method, the TMHMM method, the TMAP method, the SOSUI method, the PredictProtein method, all of which are available online through the Topology Prediction section of the expasy webserver (http://www.expasy.org).

The CRCMPs disclosed herein have been identified as soluble forms of membrane proteins, cell surface proteins, secreted proteins or GPI anchored proteins extracted from colorectal tissue samples through the methods and apparatus of the technologies described herein (generally 1D and 2D gel electrophoresis and tryptic digest of membrane proteins extracted from colorectal tissue samples). Peptide sequences were compared to the SWISS-PROT and trEMBL databases (held by the Swiss Institute of Bioinformatics (SIB) and the European Bioinformatics Institue (EBI) which are available at a http://www.expasy.com/) and the GenBank database (held by the National Institute of Health (NIH) which is available at http://www.ncbi.nlm.nih.gov/GenBank/) and corresponding genes identified. Each protein in Table 1 and Table 2 is identified by a Swiss Prot, TrEMBL or a Genbank Accession Number and each sequence is incorporated herein by reference. The apparent molecular weight and the amino acid sequences of tryptic digest peptides of these CRCMPs (Table 2) and CRCMP features (Table 1) identified by tandem mass spectrometry and database searching as described in the Examples, infra, are also listed in these Tables.

Table 3 provides further characterisation of the CRCMPs based on sample source, predictions and prior knowledge.

The proteins of the invention are useful as are fragments e.g. antigenic or immunogenic fragments thereof and derivatives thereof. Antigenic or immunogenic fragments will typically be of length 12 amino acids or more e.g. 20 amino acids or more e.g. 50 or 100 amino acids or more. Fragments may be 95% or more of the length of the full protein e.g. 90% or more e.g. 75% or 50% or 25% or 10% or more of the length of the full protein.

Antigenic or immunogenic fragments will be capable of eliciting a relevant immune response in a patient. DNA encoding the proteins of the invention are also useful as are fragments thereof e.g. DNA encoding fragments of the proteins of the invention such as immunogenic fragments thereof. Fragments of nucleic acid (e.g. DNA) encoding the proteins of the invention may be 95% or more of the length of the full coding region e.g. 90% or more e.g. 75% or 50% or 25% or 10% or more of the length of the full coding region. Fragments of nucleic acid (e.g. DNA) may be 36 nucleotides or more e.g. 60 nucleotides or more e.g. 150 or 300 nucleotides or more in length.

Derivatives of the proteins of the invention include variants on the sequences in which one or more (e.g. 1-20 such as 15 amino acids, or up to 20% such as up to 10% or 5% or 1% by number of amino acids based on the total length of the protein) deletions, insertions or substitutions have been made. Substitutions may typically be conservative substitutions. Derivatives will typically have essentially the same biological function as the protein from which they are derived. Derivatives will typically be comparably antigenic or immunogenic to the protein from which they are derived.

In one embodiment the soluble polypeptide markers of use according to the invention comprises one or more (e.g. one) amino acid sequences recited in column 4 of Table 1 (i.e. the column of tryptic digest peptides). In another embodiment the soluble polypeptides of use according to the invention comprises one or more (e.g. one) amino acid sequences recited in column 4 of Table 2 (i.e. the column of tryptic digest peptides).

Soluble peptides may typically be at least 5 amino acids in length e.g. at least 6 amino acids in length e.g. at least 10 or at least 12 or at least 15 e.g. at least 20 amino acids in length.

Suitably the marker polypeptide is derived from a protein in an isoform characterized by a pI and MW as listed in columns 2 and 3 of Table 2. An isoform is still considered to be characterized by a pI and MW as listed in columns 2 and 3 of Table 2 if the pI and MW values as determined experimentally fall within a spread of 10%, suitably 5% either side of the stated value.

Suitably the marker polypeptide will be immunologically detectable.

Certain marker polypeptides disclosed herein are novel and are claimed as an aspect of the invention.

In one embodiment, suitably assays intended to detect the marker polypeptides are configured to detect two or more said markers. Suitably the two or more said markers are derived from at least two different proteins.

In another embodiment, suitably assays intended to detect the marker polypeptides are configured to detect three or more said markers. Suitably the three or more said markers are derived from at least three different proteins.

In another embodiment, suitably assays intended to detect the marker polypeptides are configured to detect four or more said markers. Suitably the four or more said markers are derived from at least four different proteins.

In another embodiment, suitably assays intended to detect the marker polypeptides are configured to detect five or more said markers. Suitably the five or more said markers are derived from at least five different proteins.

TABLE 1
Features detected by 1D gel for the CRCMPs of the invention (see Example 1)

| | | | | |
|---|---|---|---|---|
| | MW | | | |
| CRCMP | (kDa) | Predicted | Amino Acid Sequences of Tryptic Digest Peptides | Acc. |
| # | Range | MW (Da) | [SEQ ID No] | number |

| | | | | |
|---|---|---|---|---|
| 1 | 91-126 | 92219 | AENPEPLVFGVK [37], DAYVFYAVAK [62], | Q12864 |
| | | | DEENTANSFLNYR [64], DEYGKPLSYPLEIHVK [65], | |
| | | | DINDNRPTFLQSK [68], DNVESAQASEVKPLR [70], | |
| | | | EGLLYYNR [81], GDTRGWLK [104], HTEFEER | |
| | | | [118], IDHVTGEIFSVAPLDR [119], TGAISLTR | |
| | | | [202], VSEDVALGTK [220], WNDPGAQYSLVDK | |
| | | | [227] | |
| 2 | 47-54 | 35632 | EAYEEPPEQLR [76], EGLIQWDK [80], | Q99795 |
| | | | EGSPTPQYSWK [82], EREEEDDYR [86], | |
| | | | EREEEDDYRQEEQR [87], LLLTHTER [146], | |
| | | | NYIHGELYK [165], SVTLPCTYHTSTSSR [195], | |
| | | | VTVDAISVETPQDVLR [222], | |
| | | | YNILNQEQPLAQPASGQPVSLK [240] | |
| 5 | 69-153 | 87327 | DRNHRPK [72], FGQIVNTLDK [92], | P29323 |
| | | | IPIRWTAPEAIQYR [127], MIRNPNSLK [158], | |
| | | | QLGLTEPR [170], TVAGYGRYSGK [209], | |
| | | | VSDFGLSR [219], WTAPEAIQYR [229], | |
| | | | YLADMNYVHR [237] | |
| 6 | 75-78 | 91938 | FTTPGFPDSPYPAHAR [101], GDADSVLSLTFR [103], | Q9Y5Y6 |
| | | | HPGFEATFFQLPR [117], IFQAGVVSWGDGCAQR [122], | |
| | | | SFVVTSVVAFPTDSK [189], VVMLPPR [223] | |
| 7 | 108 | 90138 | TEDVEPQSVPLLAR [200], YPPLPVDK [241] | P18433 |
| 8 | 88-104 | 112927 | ALLSDER [41], FLRPGHDPVR [95], | Q6P1M3 |
| | | | GGASELQEDESFTLR [107], QPGLVMERALLSDER | |
| | | | [171], REDVSGIASCVFTK [177], SAEDSFTGFVR | |
| | | | [183], TLYFADTYLK [206], VFEMVEALQEHPR | |
| | | | [213], VPPAERR [217], VSVAHFGSR [221], | |
| | | | YGQGFYLISPSEFER [234] | |
| 9 | 19 | 19171 | IMFVDPSLTVR [126], NLSPDGQYVPR [161] | Q8TD06 |
| 10 | 130 | 116727 | CSVPEGPFPGHLVDVR [60] | Q9UN66 |
| 12 | 42-43 | 34932 | APEFSMQGLK [44], DTEITCSER [73], EKPYDSK | P16422 |
| | | | [83], EMGEMHR [85], GESLFHSK [106], KKRMAK | |
| | | | [133], LAAKCLVMK [141], TQNDVDIADVAYYFEK | |
| | | | [207], TQNDVDIADVAYYFEKDVK [208], YEKAEIK | |
| | | | [233] | |
| 14 | 65-93 | 86705 | EGHQSEGLR [79], LQEDGLSVWFQR [151], | ENST00000322765 |
| | | | QETDYVLNNGFNPR [167], RKNLDLAAPTAEEAQR | |
| | | | [178], RPELEEIFHQYSGEDR [179] | |
| 17 | 62-72 | 55711 | LNLWISR [147], QVVEAAQAPIQER [174], | O00515 |
| | | | RSESVKSR [182] | |
| 18 | 79-96 | 82683 | AVINSAGYK [53], CPTQFPLILWHPYAR [59], | Q96TA1 |
| | | | EELCKSIQR [78], FEEVLSK [90], EQELIFEDFAR | |
| | | | [98], HEIEGTGLPQAQLLWR [114], HNLYR [116], | |
| | | | KHNLYR [132], KYDYDSSSVR [137], | |
| | | | KYDYDSSSVRK [138], KYDYDSSSVRKR [139], | |
| | | | LGEYMEK [145], MESLRLDGLQQR [156], | |
| | | | MGWMGEK [157], TDMDQIITSK [199], | |
| | | | VEGPAFTDAIR [211], VQQVQPAMQAVIR [218], | |
| | | | YDYDSSSVR [230], YDYDSSSVRK [231], | |
| | | | YDYDSSSVRKR [232] | |
| 19 | 19 | 19979 | GWGDQLIWTQTYEEALYK [112], HLSPDGQYVPR | O95994 |
| | | | [115], IMFVDPSLTVR [126], LPQTLSR [149], | |
| | | | LYAYEPADTALLLDNMK [153] | |
| 20 | 118-128 | 154374 | ATFAFSPEEQQAQR [51], AYPQYYR [55], | Q9UHN6 |
| | | | FDTHEYRNESRR [89], FRPHQDANPEKPR [99], | |
| | | | GHSPAFLQPQNGNSR [109], GYTIHWNGPAPR | |
| | | | [113], IEEYEPVHSLEELQR [120], MDNYLLR [155], | |
| | | | MPAMLTGLCQGCGTR [159], NSWQLTPR [164], | |
| | | | SDEGESMPTFGKK [184] | |
| 22 | 71-126 | 83283 | AAGSRDVSLAK [34], ADAAPDEK [35], AFVNCDENSR | P01833 |
| | | | [40], ANLTNFPENGTFVVNIAQLSQDDSGR [42], | |
| | | | AQYEGR [47], ASVDSGSSEEQGGSSR [50], | |
| | | | DGSFSVVITGLR [66], DQADGSR [71], | |
| | | | DVSLAKADAAPDE K [74], EEFVATTESTTETK [77], | |
| | | | FSSYEK [100], GGCITLISSEGYVSSK [108], | |
| | | | GSVTFHCALGPEVANVAK [111], IIEGEPNLK [123], | |
| | | | ILLNPQDK [124], KYWCR [140], | |
| | | | LSLLEEPGNGTFTVILNQLTSR [152], | |
| | | | QGHFYGETAAVYVAVEER [168], | |
| | | | QGHFYGETAAVYVAVEERK [169], | |
| | | | QSSGENCDVVVNTLGK [172], | |
| | | | QSSGENCDVVVNTLGKR [173], RAPAFEGR [175], | |
| | | | TDISMSDFENSR [198], VLDSGFR [214], | |
| | | | VLDSGFREIENK [215], VPCHFPCK [216], | |
| | | | VYTVDLGR [224], YKCGLGINSR [236], | |
| | | | YLCGAHSDGQLQEGSPIQAWQLFVNEESTIPR [238] | |
| 23 | 37 | 35964 | DYEILFK [75], FFNVLTTNTDGK [91], IEFISTMEGYK | Q92820 |
| | | | [121], NLDGISHAPNAVK [160], | |
| | | | SINGILFPGGSVDLR [190], YLESAGAR [239], | |
| | | | YPVYGVQWHPEKAPYEWK [243] | |
| 25 | 33-42 | 35463 | AEVDLQGIK [38], ASSPQGFDVDR [48], | P27216 |
| | | | ASSPQGFDVDRDAKK [49], ATFQAYQILIGK [52], | |
| | | | AYLTLVR [54], CAQDCEDYFAER [56], | |
| | | | DIEEAIEEETSGDLQK [67], DLYDAGEGR [69], | |
| | | | FQEKYQK [96], FQEKYQKSLSDMVR [97], | |
| | | | GAGTDEETLIR [102], GDTSGNLKK [105], | |
| | | | GMGTNEAAIIEILSGR [110], LFDRSLESDVK [144], | |
| | | | ILVSLLQANR [125], SDTSGDFR [186], SELSGNFEK | |
| | | | [187], SLESDVK [193], SLSDMVR [194], | |
| | | | TALALLDRPSEYAAR [197], WGTDELAFNEVLAK | |
| | | | [225], WGTDELAFNEVLAKR [226] | |
| 26 | | 29379 | SDPVTLNVR [185], TLVLLSATK [205] | Q14002 |

TABLE 2
CRCMPs detected by 2D gel (see Example 2)

| CRCMP | MW | | Amino Acid Sequences of Tryptic Digest Peptides | Acc. |
|---|---|---|---|---|
| # | (Da) | pl | [SEQ ID No] | number |
| 7 | 39716 | 5, 07 | TEDVEPQSVPLLAR [200] | P18433 |
| 7 | 40419 | 7, 88 | KFCIQQVGDMTNR [130], QAGSHSNSFR [166] | P18433 |
| 7 | 73852 | 6, 31 | QAGSHSNSFR [166] | P18433 |
| 9 | 11481 | 7, 96 | LYTYEPR [154], NLSPDGQYVPR [161], RPPQTLSR | Q8TD06 |
| | | | [180] | |
| 9 | 12984 | 8, 51 | IMFVDPSLTVR [126], LYTYEPR [154], NLSPDGQYVPR | Q8TD06 |
| | | | [161], RPPQTLSR [180] | |
| 9 | 13055 | 8, 46 | IMFVDPSLTVR [126], LYTYEPR [154], NLSPDGQYVPR | Q8TD06 |
| | | | [161], RPPQTLSR [180] | |
| 9 | 13391 | 8, 48 | FIMLNLMHETTDK [94], IMFVDPSLTVR [126], LYTYEPR | Q8TD06 |
| | | | [154], NLSPDGQYVPR [161], RPPQTLSR [180], | |
| | | | VFAQNEEIQEMAQNK [212] | |
| 9 | 14158 | 9, 96 | IMFVDPSLTVR [126] | Q8TD06 |
| 10 | 56273 | 5, 09 | AEYNVTITVTDLGTPR [39] | Q9UN66 |
| 17 | NULL | NULL | DEDEDIQSILR [63], ELEIPPR [84], KELEIPPR [129], | O00515 |
| | | | LNLWISR [147], LPDNTVK [148], LPSVEEAEVPKPLPPASK | |
| | | | [150], NLSSTTDDEAPR [162], QVVEAAQAPIQER [174], | |
| | | | RATASEQPLAQEPPASGGSPATTK [176], | |
| | | | SLAPGMALGSGR [192], TLEDEEEQER [203] | |
| 18 | NULL | NULL | AQIHMR [46], EVTDMNLNVINEGGIDK [88], | Q96TA1 |
| | | | FQELIFEDFAR [98], IVFSGNLFQHQEDSK [128], | |
| | | | VQQVQPAMQAVIR [218] | |
| 18 | NULL | NULL | FQELIFEDFAR [98], IVFSGNLFQHQEDSK [128], | Q96TA1 |
| | | | VQQVQPAMQAVIR [218] | |
| 19 | 12993 | 9, 02 | HLSPDGQYVPR [115], IMFVDPSLTVR [126] | O95994 |
| 19 | 13055 | 8, 46 | IMFVDPSLTVR [126], KVFAENK [136] | O95994 |
| 19 | 13391 | 8, 48 | IMFVDPSLTVR [126] | O95994 |
| 19 | 14158 | 9, 96 | HLSPDGQYVPR [115], IMFVDPSLTVR [126], KVFAENK | O95994 |
| | | | [136], LPQTLSR [149], LYAYEPADTALLLDNMK [153] | |
| 20 | 76700 | 5, 76 | FIGVEAGGTLELHGAR [93], TLNSSGLPFGSYTFEK [204] | Q9UHN6 |
| 22 | 58949 | 4, 65 | DGSFSVVITGLR [66] | P01833 |
| 22 | 59920 | 4, 74 | AFVNCDENSR [40], APAFEGR [43], AQYEGR [47], | P01833 |
| | | | DGSFSVVITGLR [66], FSSYEK [100], IIEGEPNLK [123], | |
| | | | ILLNPQDK [124], QGHFYGETAAVYVAVEER [168], | |
| | | | RAPAFEGR [175], YWCLWEGAQNGR [244] | |
| 22 | 64282 | 5, 05 | APAFEGR [43], DGSFSVVITGLR [66], IIEGEPNLK [123], | P01833 |
| | | | RAPAFEGR [175], VYTVDLGR [224] | |
| 22 | 72124 | 5, 15 | APAFEGR [43], DGSFSVVITGLR [66], IIEGEPNLK [123], | P01833 |
| | | | QGHFYGETAAVYVAVEER [168], RAPAFEGR [175], | |
| | | | TENAQKR [201], VLDSGFR [214], VYTVDLGR [224] | |
| 22 | 72683 | 5, 03 | AFVNCDENSR [40], ANLTNFPENGTFVVNIAQLSQDDSGR [42], | P01833 |
| | | | APAFEGR [43], AQYEGR [47], CGLGINSR [57], | |
| | | | CPLLVDSEGWVK [58], DGSFSVVITGLR [66], FSSYEK [100], | |
| | | | IIEGEPNLK [123], ILLNPQDK [124], QGHFYGETAAVYVAVEER | |
| | | | [168], QSSGENCDVVVNTLGK [172], RAPAFEGR [175], | |
| | | | VLDSGFR [214], VYTVDLGR [224], YWCLWEGAQNGR [244] | |
| 22 | 73988 | 4, 96 | AFVNCDENSR [40], APAFEGR [43], AQYEGR [47], | P01833 |
| | | | CGLGINSR [57], DGSFSVVITGLR [66], FSSYEK [100], | |
| | | | IIEGEPNLK [123], ILLNPQDK [124], QGHFYGETAAVYVAVEER | |
| | | | [168], QSSGENCDVVVNTLGK [172], RAPAFEGR [175], | |
| | | | TVTINCPFK [210], VLDSGFR [214], VYTVDLGR [224], | |
| | | | YLCGAHSDGQLQEGSPIQAWQLFVNEESTIPR [238], | |
| | | | YWCLWEGAQNGR [244] | |
| 22 | 76022 | 5, 63 | AFVNCDENSR [40], APAFEGR [43], AQYEGR [47], | P01833 |
| | | | CGLGINSR [57], DGSFSVVITGLR [66], FSSYEK [100], | |
| | | | IIEGEPNLK [123], QGHFYGETAAVYVAVEER [168], | |
| | | | QSSGENCDVVVNTLGK [172], RAPAFEGR [175], | |
| | | | TENAQKR [201], VLDSGFR [214], VPCHFPCK [216], | |
| | | | VYTVDLGR [224], YWCLWEGAQNGR [244] | |
| 22 | 76452 | 5, 02 | AFVNCDENSR [40], APAFEGR [43], AQYEGR [47], | P01833 |
| | | | CGLGINSR [57], CPLLVDSEGWVK [58], | |
| | | | DAGFYWCLTNGDTLWR [61], DGSFSVVITGLR [66], | |
| | | | FSSYEK [100], IIEGEPNLK [123], ILLNPQDK [124], | |
| | | | KYWCR [140], LDIQGTGQLLFSVVINQLR [142], | |
| | | | QGHFYGETAAVYVAVEER [168], QSSGENCDVVVNTLGK [172], | |
| | | | RAPAFEGR [175], VLDSGFR [214], VYTVDLGR [224], | |
| | | | YLCGAHSDGQLQEGSPIQAWQLFVNEESTIPR [238], | |
| | | | YWCLWEGAQNGR [244] | |
| 22 | 76788 | 5, 09 | AFVNCDENSR [40], APAFEGR [43], AQYEGR [47], | P01833 |
| | | | CGLGINSR [57], DGSFSVVITGLR [66], FSSYEK [100], | |
| | | | IIEGEPNLK [123], ILLNPQDK [124], KYWCR [140], | |
| | | | LDIQGTGQLLFSVVINQLR [142], LSLLEEPGNGTFTVILNQLTSR | |
| | | | [152], QGHFYGETAAVYVAVEER [168], QSSGENCDVVVNTLGK | |
| | | | [172], RAPAFEGR [175], VLDSGFR [214], VYTVDLGR | |
| | | | [224], YLCGAHSDGQLQEGSPIQAWQLFVNEESTIPR [238] | |
| 22 | 76811 | 5, 20 | AFVNCDENSR [40], APAFEGR [43], AQYEGR [47], | P01833 |
| | | | CGLGINSR [57], DGSFSVVITGLR [66], FSSYEK [100], | |
| | | | IIEGEPNLK [123], ILLNPQDK [124], KYWCR [140], | |
| | | | LDIQGTGQLLFSVVINQLR [142], | |
| | | | QGHFYGETAAVYVAVEER [168], RAPAFEGR [175], | |
| | | | VYTVDLGR [224], YLCGAHSDGQLQEGSPIQAWQLFVNEESTIPR | |
| | | | [238], YWCLWEGAQNGR [244] | |
| 22 | 76905 | 4, 84 | AFVNCDENSR [40], APAFEGR [43], AQYEGR [47], | P01833 |
| | | | CGLGINSR [57], DGSFSVVITGLR [66], FSSYEK [100], | |
| | | | IIEGEPNLK [123], ILLNPQDK [124], | |
| | | | QGHFYGETAAVYVAVEER [168], RAPAFEGR [175], | |
| | | | VLDSGFR [214], VYTVDLGR [224], | |
| | | | YLCGAHSDGQLQEGSPIQAWQLFVNEESTIPR [238], | |
| | | | YWCLWEGAQNGR [244] | |
| 22 | 77049 | 5, 03 | AFVNCDENSR [40], APAFEGR [43], CGLGINSR [57], | P01833 |
| | | | DGSFSVVITGLR [66], FSSYEK [100], IIEGEPNLK [123], | |
| | | | QGHFYGETAAVYVAVEER [168], RAPAFEGR [175], | |
| | | | VYTVDLGR [224] | |
| 22 | 77219 | 5, 09 | AFVNCDENSR [40], APAFEGR [43], CGLGINSR [57], | P01833 |
| | | | DGSFSVVITGLR [66], IIEGEPNLK [123], | |
| | | | QGHFYGETAAVYVAVEER [168], RAPAFEGR [175], | |
| | | | VYTVDLGR [224] | |
| 22 | 77291 | 5, 63 | AFVNCDENSR [40], ANLTNFPENGTFVVNIAQLSQDDSGR [42], | P01833 |
| | | | APAFEGR [43], AQYEGR [47], CGLGINSR [57], | |
| | | | DGSFSVVITGLR [66], FSSYEK [100], IIEGEPNLK [123], | |
| | | | LFAEEK [143], QGHFYGETAAVYVAVEER [168], | |
| | | | QSSGENCDVVVNTLGK [172], RAPAFEGR [175], | |
| | | | VLDSGFR [214], VYTVDLGR [224], YWCLWEGAQNGR [244] | |
| 22 | 77900 | 4, 80 | AFVNCDENSR [40], ANLTNFPENGTFVVNIAQLSQDDSGR [42], | P01833 |
| | | | APAFEGR [43], AQYEGR [47], CGLGINSR [57], | |
| | | | DGSFSVVITGLR [66], FSSYEK [100], IIEGEPNLK [123], | |
| | | | ILLNPQDK [124], KYWCR [140], QGHFYGETAAVYVAVEER | |
| | | | [168], QSSGENCDVVVNTLGK [172], RAPAFEGR [175], | |
| | | | VLDSGFR [214], VYTVDLGR [224], | |
| | | | YLCGAHSDGQLQEGSPIQAWQLFVNEESTIPR [238], | |
| | | | YWCLWEGAQNGR [244] | |
| 22 | 77980 | 5, 00 | ADEGWYWCGVK [36], AFVNCDENSR [40], APAFEGR [43], | P01833 |
| | | | AQYEGR [47], CGLGINSR [57], CPLLVDSEGWVK [58], | |
| | | | DGSFSVVITGLR [66], FSSYEK [100], | |
| | | | GSVTFHCALGPEVANVAK [111], IIEGEPNLK [123], | |
| | | | ILLNPQDK [124], KNADLQVLKPEPELVYEDLR [134], | |
| | | | KYWCR [140], QGHFYGETAAVYVAVEER [168], | |
| | | | RAPAFEGR [175], TVTINCPFK [210], VLDSGFR [214], | |
| | | | VYTVDLGR [224], YWCLWEGAQNGR [244] | |
| 22 | 79500 | 4, 91 | ADEGWYWCGVK [36], AFVNCDENSR [40], APAFEGR [43], | P01833 |
| | | | AQYEGR [47], CGLGINSR [57], CPLLVDSEGWVK [58], | |
| | | | DGSFSVVITGLR [66], FSSYEK [100], GGCITLISSEGYVSSK | |
| | | | [108], GSVTFHCALGPEVANVAK [111], IIEGEPNLK [123], | |
| | | | ILLNPQDK [124], KNADLQVLKPEPELVYEDLR [134], KYWCR | |
| | | | [140], QGHFYGETAAVYVAVEER [168], QSSGENCDVVVNTLGK | |
| | | | [172], RAPAFEGR [175], TENAQKR [201], VLDSGFR [214], | |
| | | | VYTVDLGR [224], YLCGAHSDGQLQEGSPIQAWQLFVNEESTIPR | |
| | | | [238], YWCLWEGAQNGR [244] | |
| 22 | 79705 | 5, 05 | AFVNCDENSR [40], APAFEGR [43], AQYEGR [47], | P01833 |
| | | | CGLGINSR [57], DGSFSVVITGLR [66], | |
| | | | QGHFYGETAAVYVAVEER [168], RAPAFEGR [175], | |
| | | | VLDSGFR [214], VYTVDLGR [224], | |
| | | | YLCGAHSDGQLQEGSPIQAWQLFVNEESTIPR [238] | |
| 22 | 80272 | 5, 97 | AFVNCDENSR [40], APAFEGR [43], AQYEGR [47], | P01833 |
| | | | CGLGINSR [57], DGSFSVVITGLR [66], FSSYEK [100], | |
| | | | IIEGEPNLK [123], ILLNPQDK [124], | |
| | | | LDIQGTGQLLFSVVINQLR [142], QGHFYGETAAVYVAVEER | |
| | | | [168], RAPAFEGR [175], VLDSGFR [214], VYTVDLGR | |
| | | | [224], YLCGAHSDGQLQEGSPIQAWQLFVNEESTIPR [238], | |
| | | | YWCLWEGAQNGR [244] | |
| 22 | 80654 | 5, 02 | ANLTNFPENGTFVVNIAQLSQDDSGR [42], DGSFSVVITGLR [66], | P01833 |
| | | | QGHFYGETAAVYVAVEER [168], RAPAFEGR [175], VLDSGFR | |
| | | | [214], VYTVDLGR [224] | |
| 22 | 80735 | 5, 78 | AFVNCDENSR [40], APAFEGR [43], AQYEGR [47], | P01833 |
| | | | CGLGINSR [57], DGSFSVVITGLR [66], FSSYEK [100], | |
| | | | IIEGEPNLK [123], ILLNPQDK [124], QGHFYGETAAVYVAVEER | |
| | | | [168], RAPAFEGR [175], VLDSGFR [214], VYTVDLGR | |
| | | | [224], YLCGAHSDGQLQEGSPIQAWQLFVNEESTIPR [238], | |
| | | | YWCLWEGAQNGR [244] | |
| 22 | 83246 | 5, 15 | AFVNCDENSR [40], ANLTNFPENGTFVVNIAQLSQDDSGR [42], | P01833 |
| | | | APAFEGR [43], AQYEGR [47], CGLGINSR [57], | |
| | | | DGSFSVVITGLR [66], FSSYEK [100], IIEGEPNLK [123], | |
| | | | ILLNPQDK [124], QGHFYGETAAVYVAVEER [168], RAPAFEGR | |
| | | | [175], VLDSGFR [214], VYTVDLGR [224] | |
| 22 | 83366 | 4, 72 | AFVNCDENSR [40], ANLTNFPENGTFVVNIAQLSQDDSGR [42], | P01833 |
| | | | APAFEGR [43], AQYEGR [47], CGLGINSR [57], | |
| | | | DGSFSVVITGLR [66], FSSYEK [100], IIEGEPNLK [123], | |
| | | | ILLNPQDK [124], KYWCR [140], QGHFYGETAAVYVAVEER | |
| | | | [168], QSSGENCDWVNTLGK [172], RAPAFEGR [175], | |
| | | | VLDSGFR [214], VYTVDLGR [224], | |
| | | | YLCGAHSDGQLQEGSPIQAWQLFVNEESTIPR [238], | |
| | | | YWCLWEGAQNGR [244] | |
| 22 | 83750 | 4, 96 | APAFEGR [43], AQYEGR [47], DGSFSVVITGLR [66], | P01833 |
| | | | FSSYEK [100], IIEGEPNLK [123], QGHFYGETAAVYVAVEER | |
| | | | [168], RAPAFEGR [175], VLDSGFR [214], VYTVDLGR [224] | |
| 22 | 83905 | 5, 07 | APAFEGR [43], AQYEGR [47], DGSFSVVITGLR [66], | P01833 |
| | | | QGHFYGETAAVYVAVEER [168], RAPAFEGR [175], | |
| | | | VLDSGFR [214], VYTVDLGR [224] | |
| 22 | 84555 | 5, 07 | DGSFSVVITGLR [66], QGHFYGETAAVYVAVEER [168], | P01833 |
| | | | RAPAFEGR [175], VLDSGFR [214] | |
| 22 | 84742 | 4, 90 | AFVNCDENSR [40], APAFEGR [43], AQYEGR [47], | P01833 |
| | | | DGSFSVVITGLR [66], IIEGEPNLK [123], | |
| | | | LDIQGTGQLLFSVVINQLR [142], | |
| | | | QGHFYGETAAVYVAVEER [168], RAPAFEGR [175], | |
| | | | VLDSGFR [214], VYTVDLGR [224] | |
| 22 | 86180 | 4, 86 | AFVNCDENSR [40], APAFEGR [43], AQYEGR [47], | P01833 |
| | | | CGLGINSR [57], DGSFSVVITGLR [66], FSSYEK [100], | |
| | | | IIEGEPNLK [123], QGHFYGETAAVYVAVEER [168], | |
| | | | RAPAFEGR [175], VLDSGFR [214], VYTVDLGR [224] | |
| 22 | 90403 | 4, 78 | AFVNCDENSR [40], APAFEGR [43], AQYEGR [47], | P01833 |
| | | | DGSFSVVITGLR [66], FSSYEK [100], IIEGEPNLK [123], | |
| | | | QGHFYGETAAVYVAVEER [168], RAPAFEGR [175], | |
| | | | VLDSGFR [214], VYTVDLGR [224] | |
| 22 | 91105 | 4, 74 | DGSFSVVITGLR [66], LDIQGTGQLLFSVVINQLR [142], | P01833 |
| | | | QGHFYGETAAVYVAVEER [168], RAPAFEGR [175], | |
| | | | VLDSGFR [214], VYTVDLGR [224] | |
| 22 | 92925 | 4, 74 | APAFEGR [43], DGSFSVVITGLR [66], QGHFYGETAAVYVAVEER | P01833 |
| | | | [168], RAPAFEGR [175], VLDSGFR [214], VYTVDLGR [224] | |
| 23 | 32654 | 5, 31 | APYEWK [45], DYEILFK [75], FFNVLTTNTDGK [91], | Q92820 |
| | | | IEFISTMEGYK [121], KNNHHFK [135], NLDGISHAPNAVK | |
| | | | [160], SINGILFPGGSVDLR [190], TAFYLAEFFVNEAR [196], | |
| | | | WSLSVK [228], YLESAGAR [239], YPVYGVQWHPEK [242], | |
| | | | YYIAASYVK [245] | |
| 23 | 32772 | 5, 46 | APYEWK [45], NLDGISHAPNAVK [160], TAFYLAEFFVNEAR | Q92820 |
| | | | [196], YLESAGAR [239], YPVYGVQWHPEK [242] | |
| 23 | 33240 | 5, 56 | IEFISTMEGYK [121], SINGILFPGGSVDLR [190], | Q92820 |
| | | | TAFYLAEFFVNEAR [196] | |
| 23 | 33503 | 5, 50 | APYEWK [45], DYEILFK [75], FFNVLTTNTDGK [91], | Q92820 |
| | | | IEFISTMEGYK [121], KFFNVLTTNTDGK [131], | |
| | | | KNNHHFK [135], NLDGISHAPNAVK [160], RSDYAK [181], | |
| | | | SESEEEK [188], SINGILFPGGSVDLR [190], | |
| | | | TAFYLAEFFVNEAR [196], WSLSVK [228], YLESAGAR [239], | |
| | | | YPVYGVQWHPEK [242], YYIAASYVK [245] | |
| 23 | 34247 | 5, 32 | APYEWK [45], DYEILFK [75], FFNVLTTNTDGK [91], | Q92820 |
| | | | IEFISTMEGYK [121], KFFNVLTTNTDGK [131], | |
| | | | KNNHHFK [135], NLDGISHAPNAVK [160], NNHHFK [163], | |
| | | | RSDYAK [181], SESEEEK [188], SINGILFPGGSVDLR [190], | |
| | | | TAFYLAEFFVNEAR [196], WSLSVK [228], YLESAGAR [239], | |
| | | | YPVYGVQWHPEK [242], YYIAASYVK [245] | |
| 23 | 34827 | 5, 20 | APYEWK [45], DYEILFK [75], FFNVLTTNTDGK [91], | Q92820 |
| | | | IEFISTMEGYK [121], KNNHHFK [135], NLDGISHAPNAVK | |
| | | | [160], SINGILFPGGSVDLR [190], YLESAGAR [239], | |
| | | | YPVYGVQWHPEK [242], YYIAASYVK [245] | |
| 23 | 34996 | 5, 01 | APYEWK [45], DYEILFK [75], FFNVLTTNTDGK [91], | Q92820 |
| | | | IEFISTMEGYK [121], KNNHHFK [135], NLDGISHAPNAVK | |
| | | | [160], NNHHFK [163], SINGILFPGGSVDLR [190], | |
| | | | TAFYLAEFFVNEAR [196], YLESAGAR [239], YPVYGVQWHPEK | |
| | | | [242], YYIAASYVK [245] | |
| 23 | 35025 | 5, 42 | APYEWK [45], DYEILFK [75], IEFISTMEGYK [121], | Q92820 |
| | | | NLDGISHAPNAVK [160], SINGILFPGGSVDLR [190], | |
| | | | SINGILFPGGSVDLRR [191], TAFYLAEFFVNEAR [196], | |
| | | | YLESAGAR [239], YPVYGVQWHPEK [242] | |

TABLE 3
CRCMP Categories

| | Trans | Known | | |
|---|---|---|---|---|
| | Membrane | Truncated | GPI Anchored | Secreted |
| CRCMP # | Type | Isoforms | Cell Surface | Isoform |
| 1 | I | | | |
| 2 | I | | | |
| 5 | I | | | |
| 6 | II | | | |
| 7 | I | yes | | |
| 8 | unknown | yes | | |
| 9 | | | | yes |
| 10 | I | yes | | |
| 12 | I | | | |
| 14 | | | Probable | |
| 17 | | yes | | yes |
| 18 | unknown | yes | | |
| 19 | | yes | | yes |
| 20 | unknown | yes | | |
| 22 | I | yes | | yes |
| 23 | | yes | | yes |
| 25 | unknown | | | |
| 26 | | yes | yes | |

Membrane proteins come in numerous types with a few different suggested classifications. One of the most commonly used to date is the classification method suggested by JS Singer: Type I proteins have a single TM stretch of hydrophobic residues, with the portion of the polypeptide on the NH2-terminal side of the TM domain exposed on the exterior side of the membrane and the COOH-terminal portion exposed on the cytoplasmic side. The proteins are subdivided into types Ia (cleavable signal sequences) and Ib (without cleavable signal sequence). Most eukaryotic mebrane proteins with single spanning regions are of Type Ia. Type II membrane proteins are similar to the type I class in that they span the membrane only once, but they have their amino terminus on the cytoplasmic side of the cell and the carboxy terminus on the exterior. Type III membrane proteins have multiple transmembrane domains in a single polypeptide chain. They are also sub divided into a and b: Type IIIa molecules have cleavable signal sequences while type IIIb have their amino termini exposed on the exterior surface of the membrane, but do not have a cleavable signal sequences. Type IIIa proteins include the M and L peptides of the photoreaction center. Type IIIb proteins include e.g. cytochrome P450, and leader peptidase of E. coli. Type IV proteins have multiple homologous domains which make up an assembly that spans the membrane multiple times. The domains may reside on a single polypeptide chain or be on more than one individual chain. This nomenclature is used in Table 3.

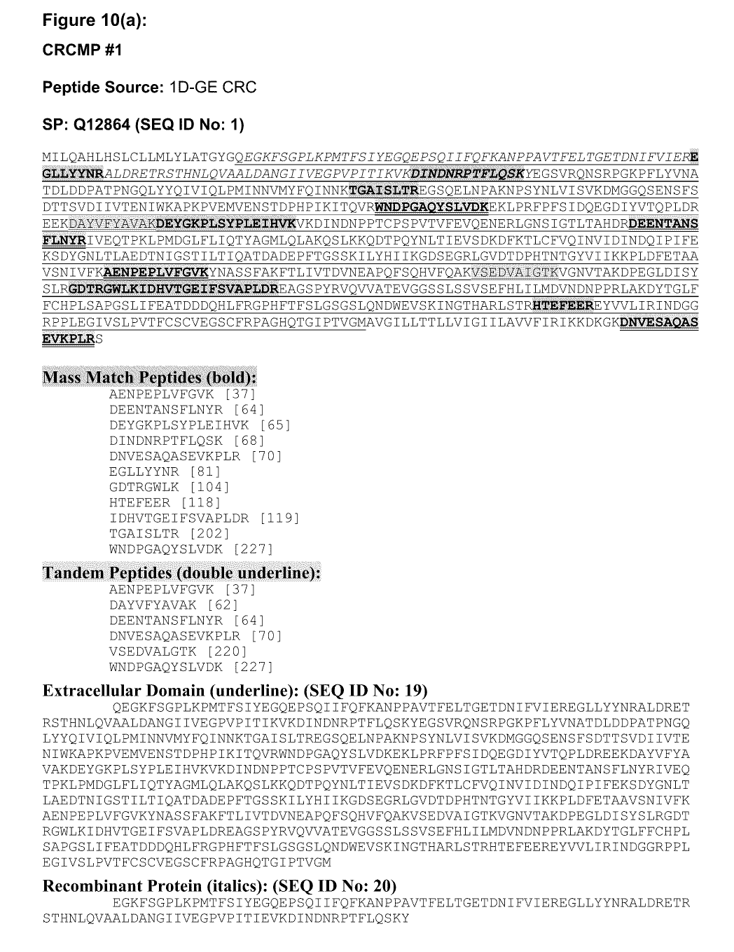

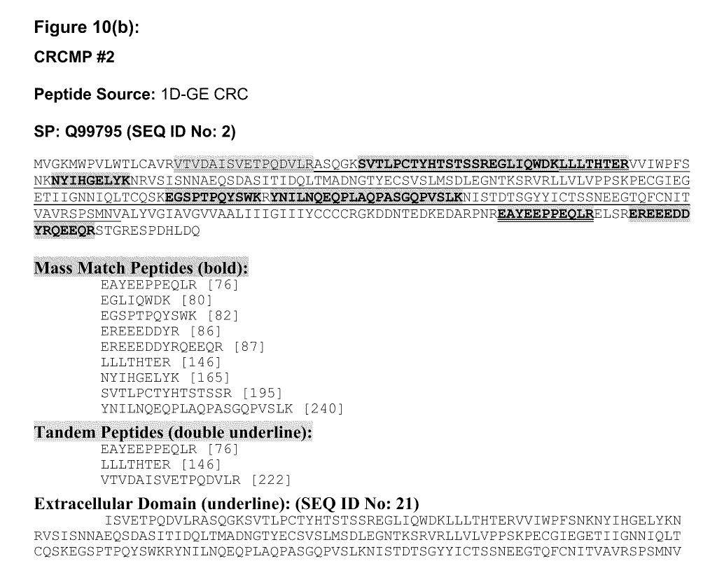

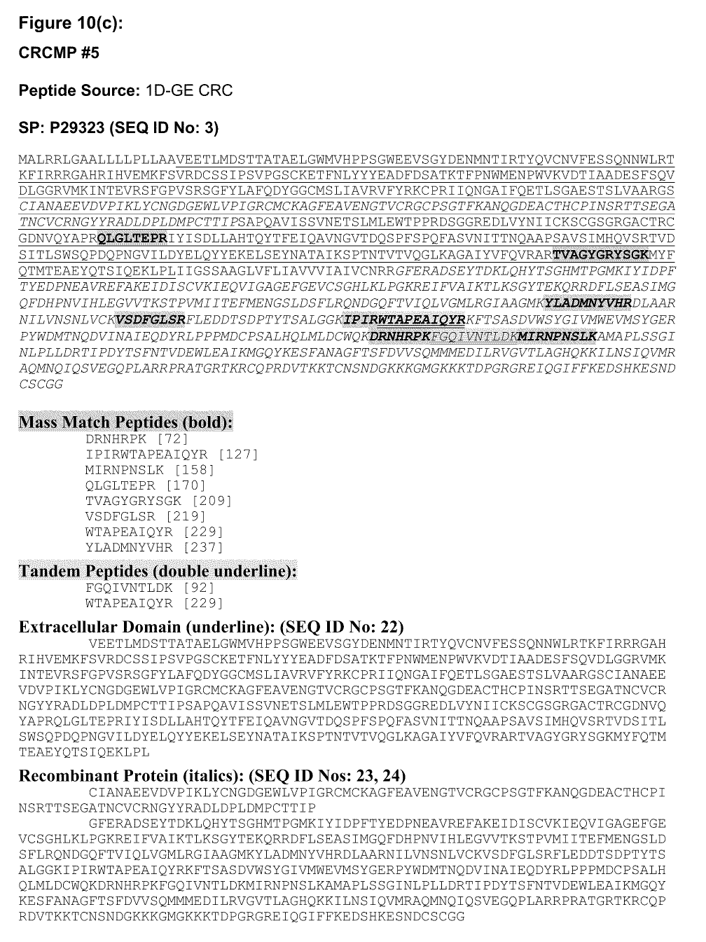

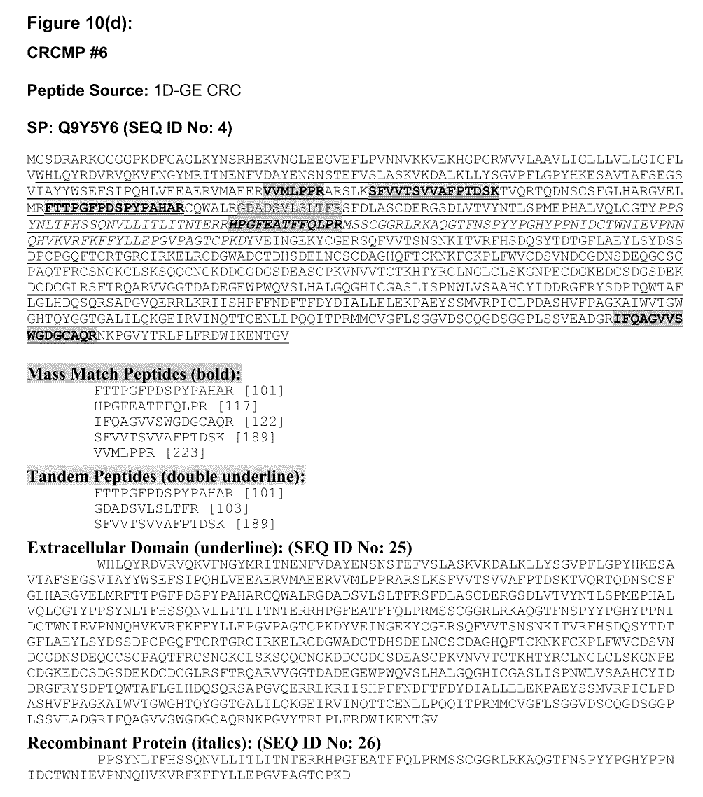

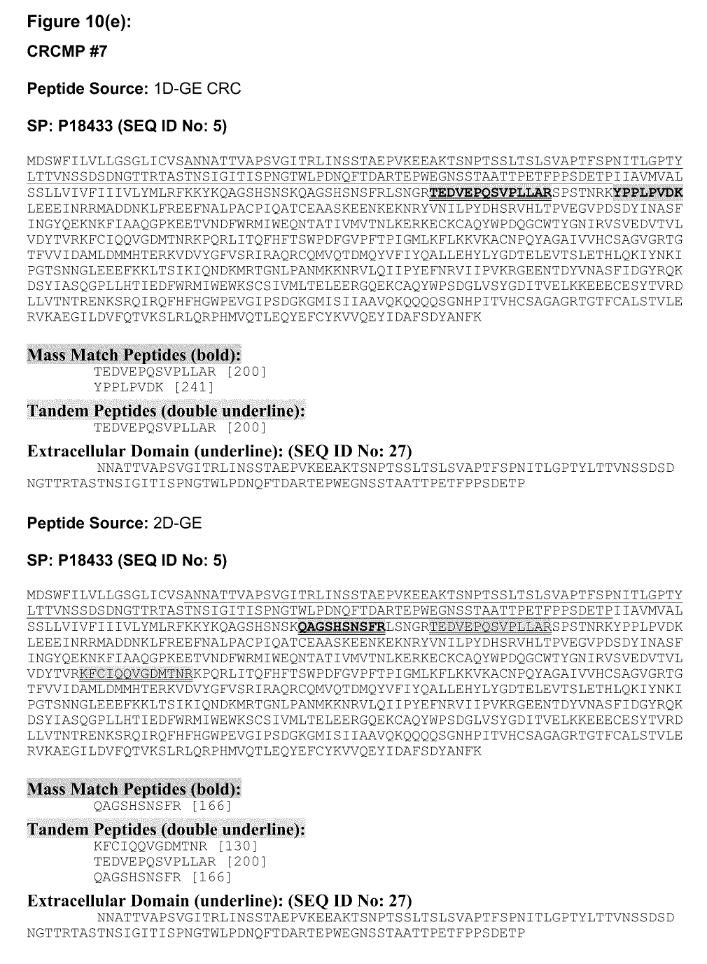

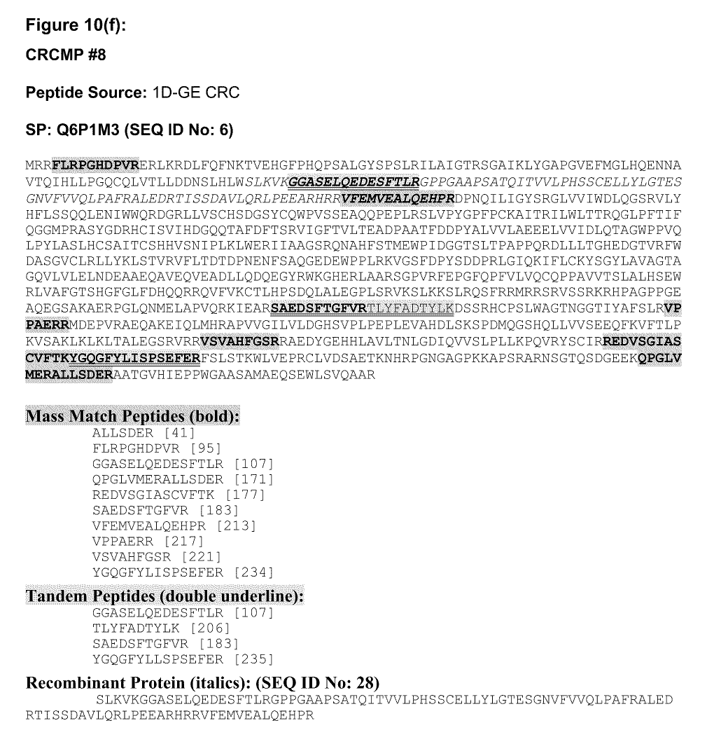

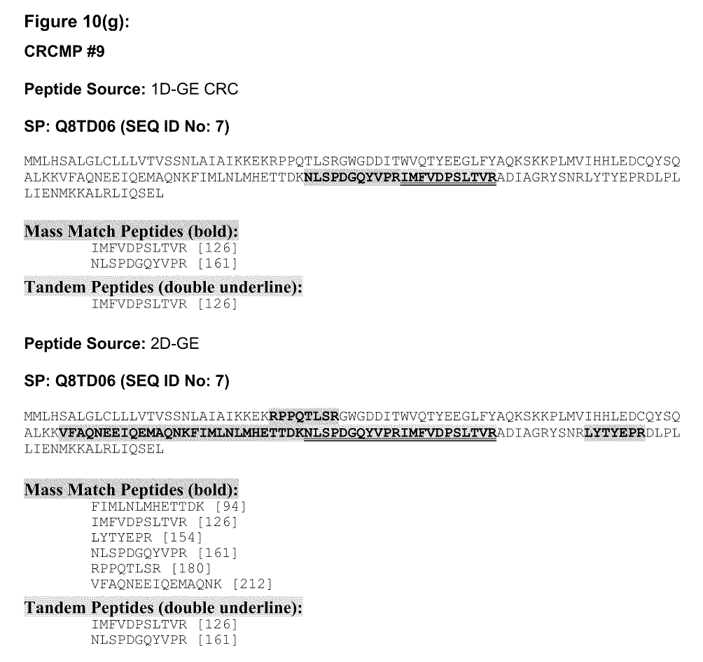

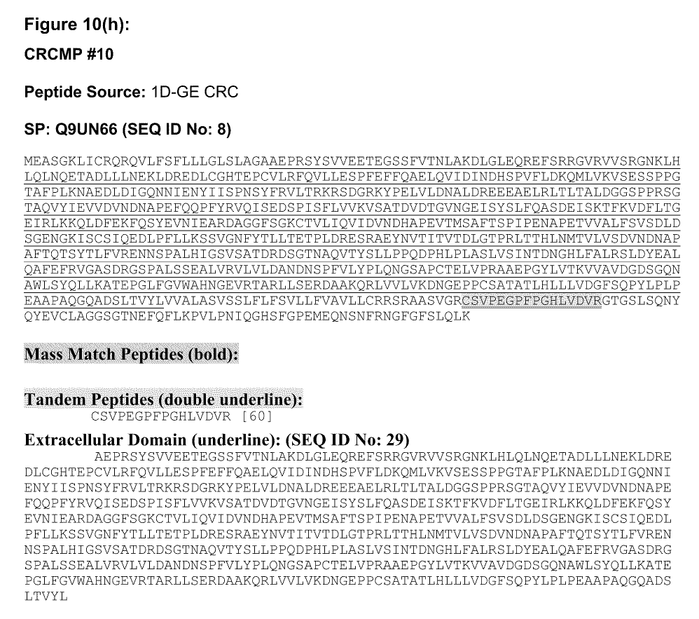

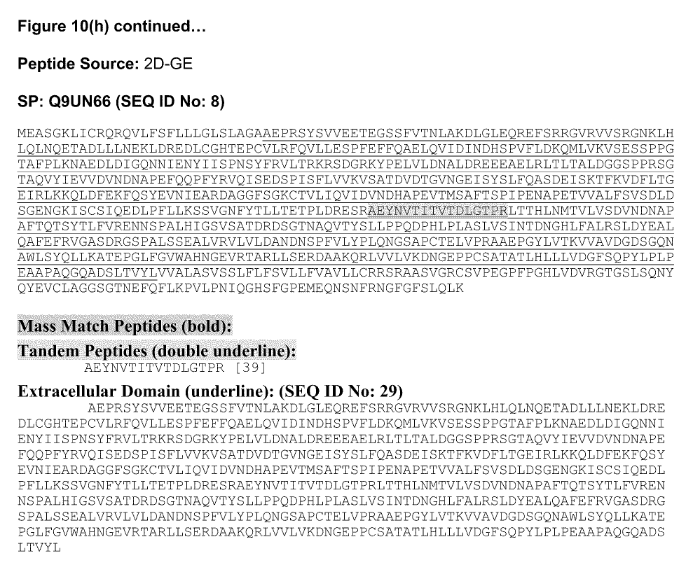

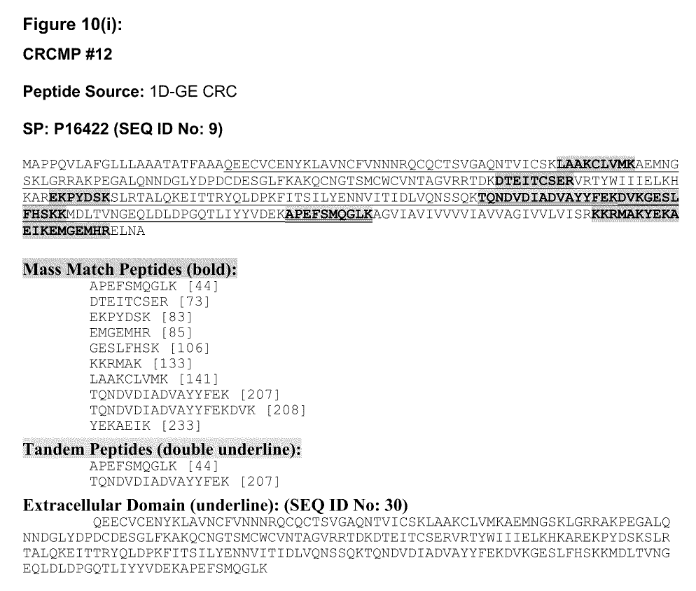

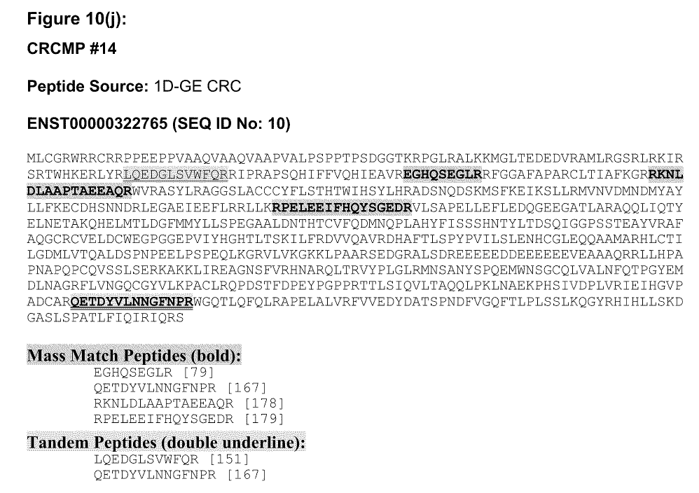

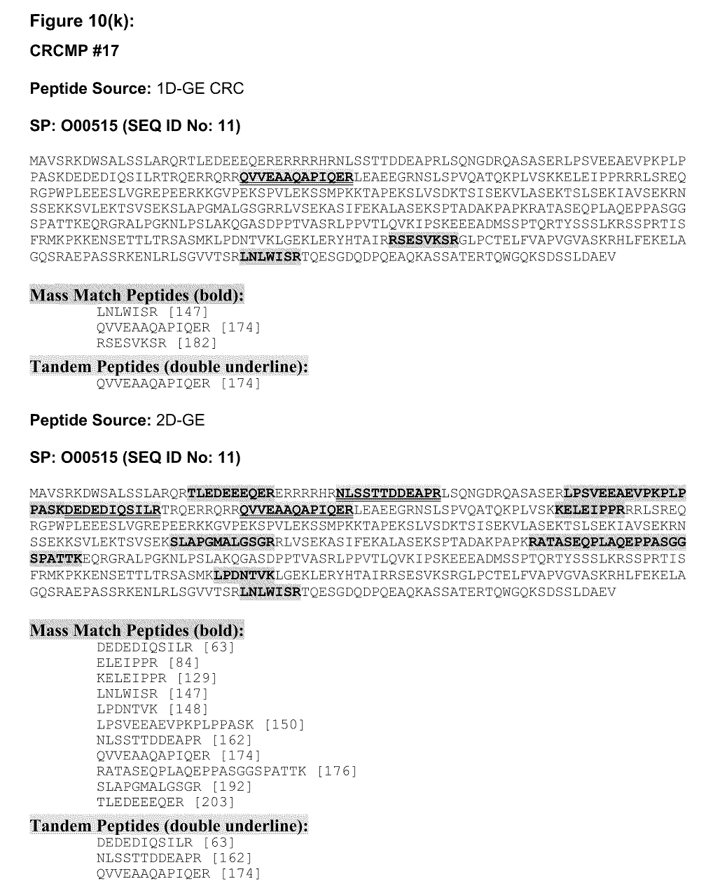

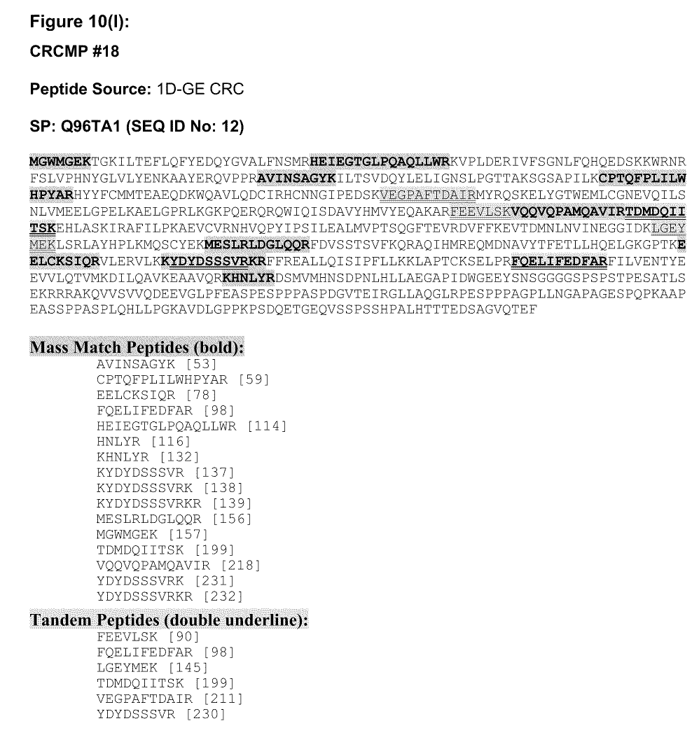

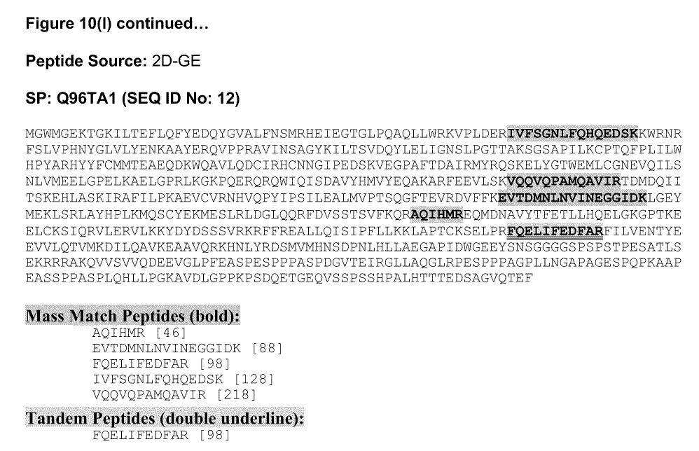

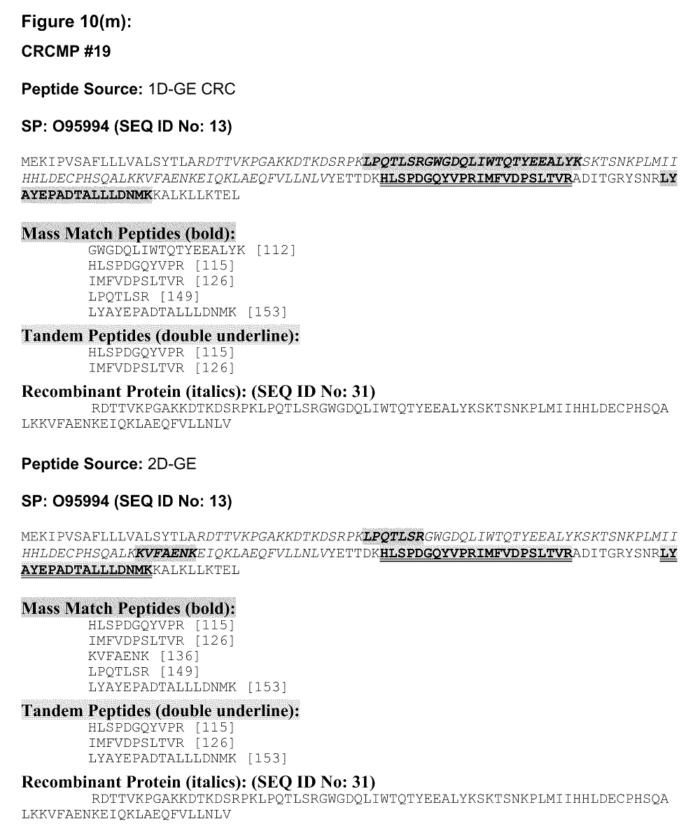

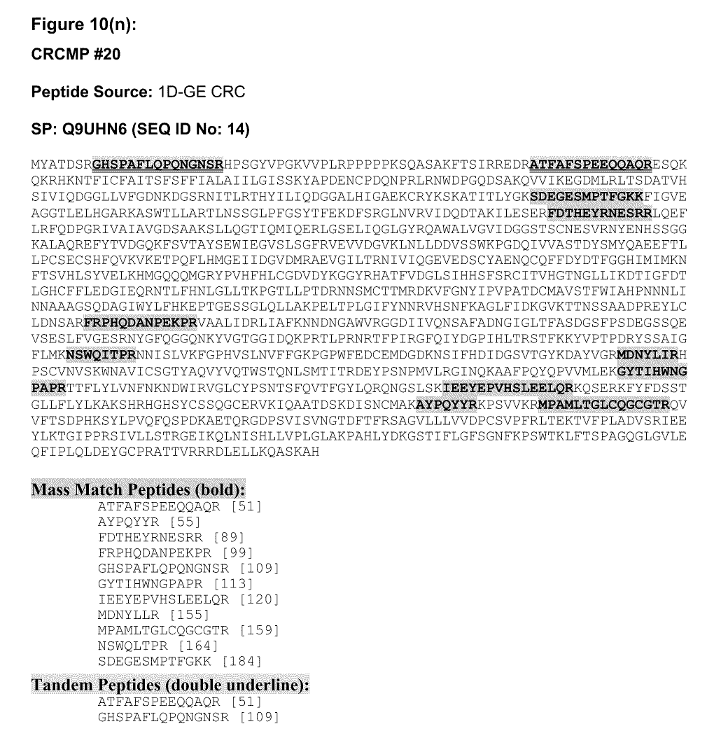

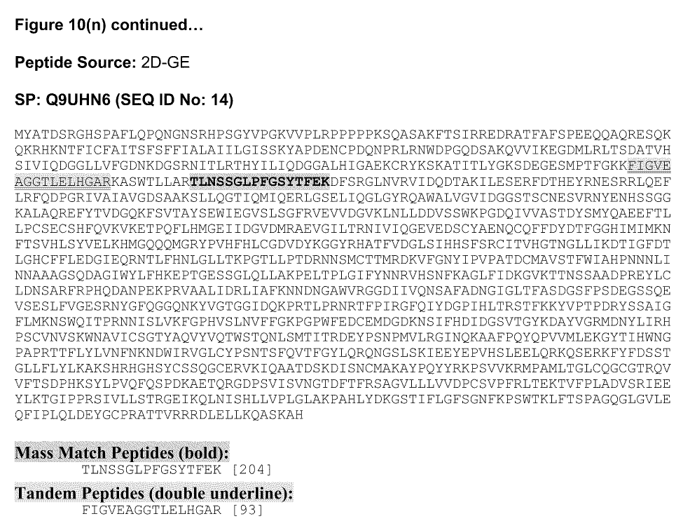

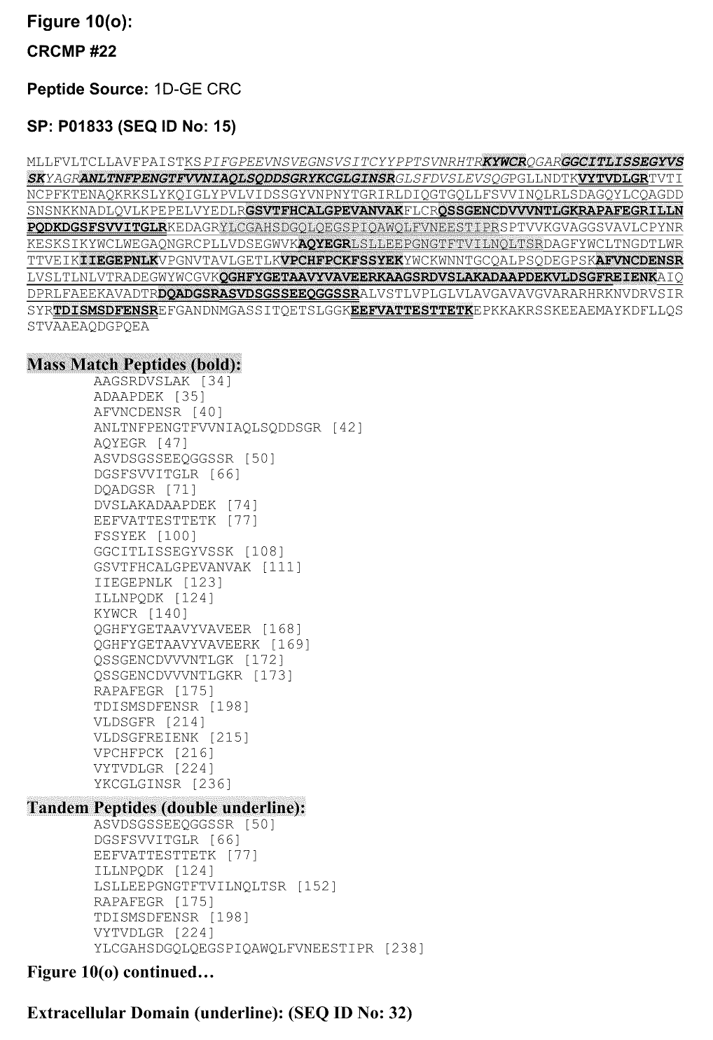

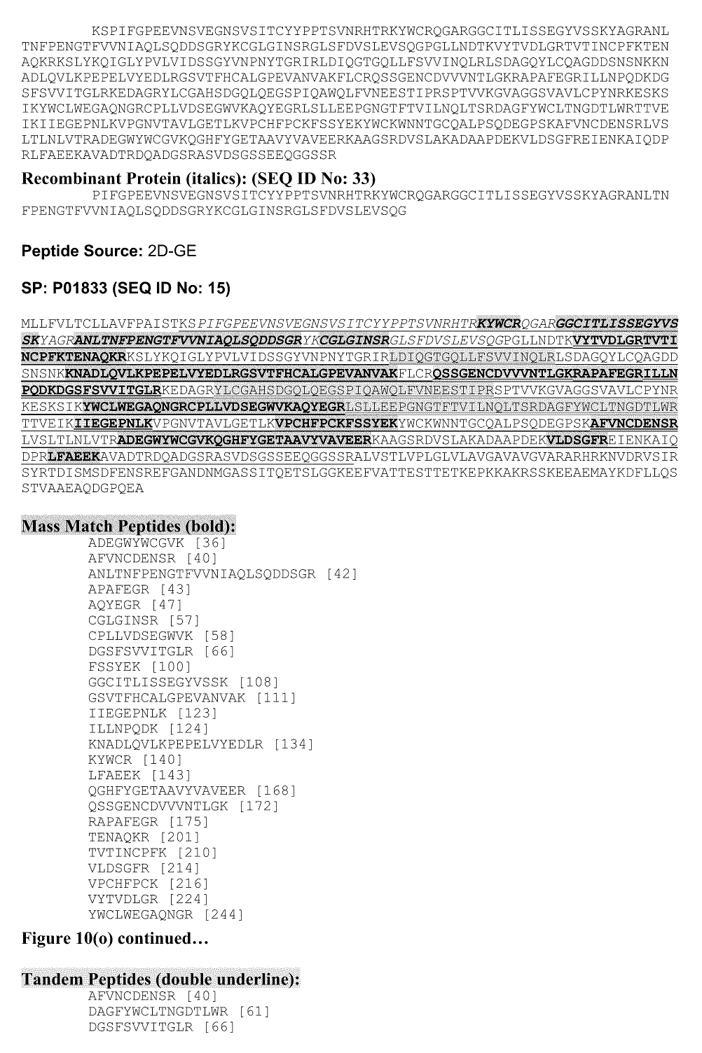

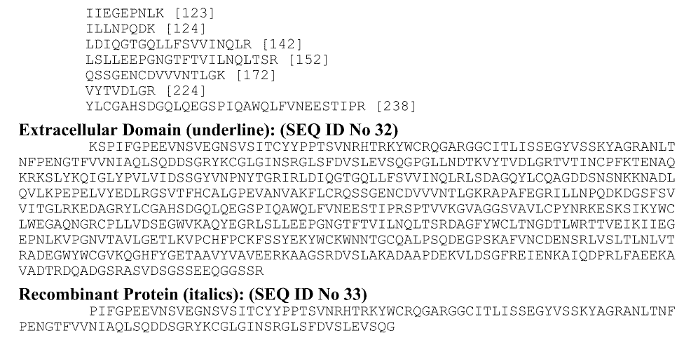

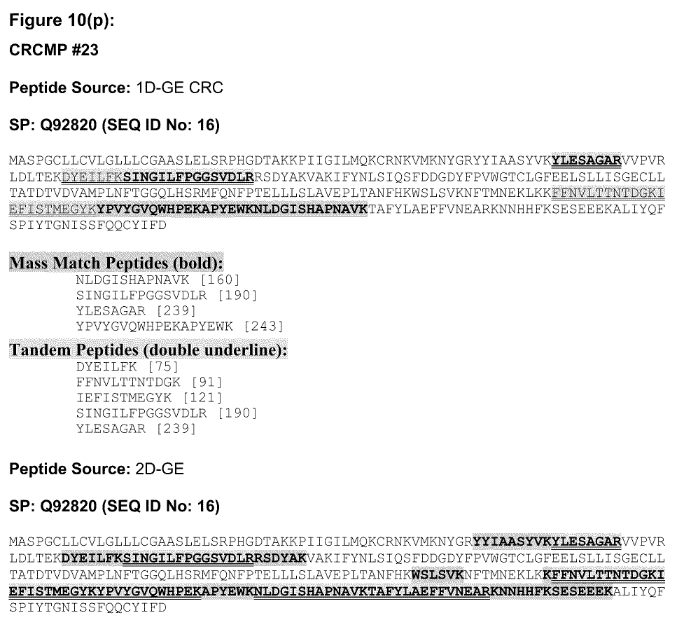

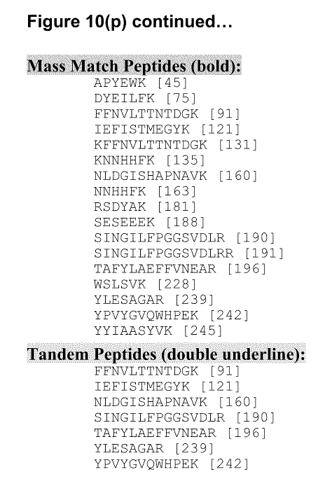

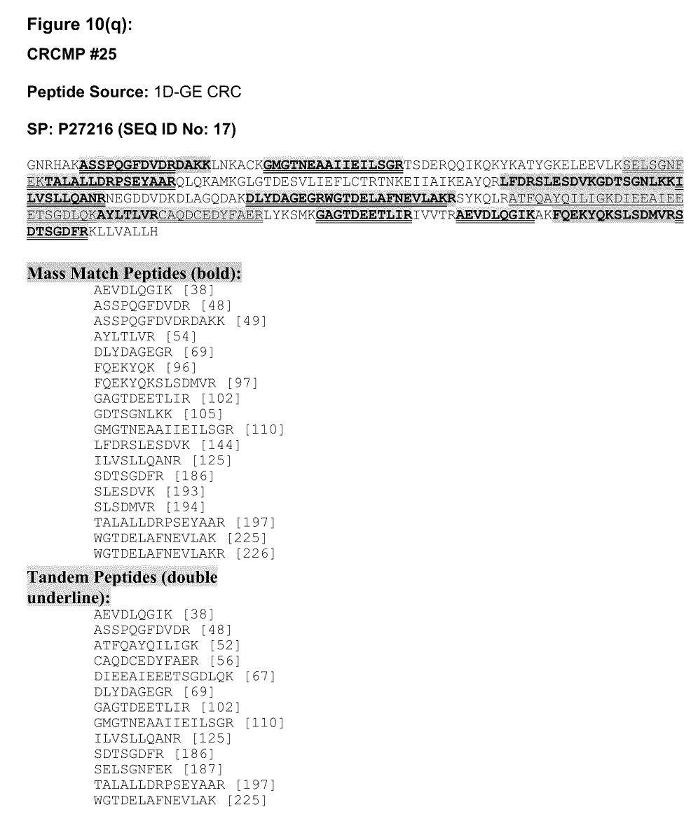

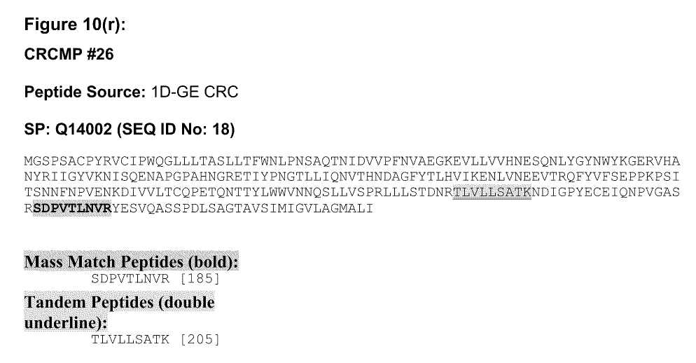

The sequences of the 18 proteins referred to in Table 1 and 2 are recited in FIGS. 10(a) to (r). The portions of the sequence which correspond to the Mass Match Peptides are shown in bold. The portions of the sequence which correspond to the Tandem Peptides are shown in double underline. The portion(s) of the sequences which correspond to an extracellular part of the whole protein are shown in underline (SEQ ID Nos 19, 21, 22, 25, 27, 29, 30 and 32). Preferred soluble peptides/CRCMPs according to the invention have sequences which overlap with or are preferably within an extracellular part of the whole protein.

Portions of the sequence which correspond to commercially available recombinant proteins are shown in italics (SEQ ID Nos 20, 23, 24, 26, 28, 31 and 33). These may, for example, be readily employed to raise antibodies for use according to the invention, especially when they overlap with or are preferably within the extracellular part of the whole protein. Other non-commercially available portions of the whole protein or, other soluble polypeptides according to the invention, may be prepared using conventional methods known to a skilled person e.g. expression of protein in a host cell containing a suitable vector (bacterial or mammalian system) or by stepwise peptide synthesis.

For any given CRCMP, the detected level obtained upon analyzing serum from subjects having colorectal cancer relative to the detected level obtained upon analyzing serum from subjects free from colorectal cancer will depend upon the particular analytical protocol and detection technique that is used, provided that such CRCMP is differentially expressed between normal and disease tissue. Accordingly, the present invention contemplates that each laboratory will establish a reference range for each CRCMP in subjects free from colorectal cancer according to the analytical protocol and detection technique in use, as is conventional in the diagnostic art. Preferably, at least one control positive serum sample from a subject known to have colorectal cancer or at least one control negative serum sample from a subject known to be free from colorectal cancer (and more preferably both positive and negative control samples) are included in each batch of test samples analysed.

In an assay the objective may be to detect the presence of a marker polypeptide. Alternatively it may be to determine the level of a marker polypeptide. Assay design may provide for an appropriate threshold of detection such that detection of a marker polypeptide can be correlated with detection of a specified level of that polypeptide.

In one embodiment, the level of expression of a protein is determined relative to a background value, which is defined as the level of signal obtained from a proximal region of the image that (a) is equivalent in area to the particular feature in question; and (b) contains no discernable protein feature.

CRCMPs can be used for detection, prognosis, diagnosis, or monitoring of colorectal cancer or for drug development. In one embodiment of the invention, serum from a subject (e.g., a subject suspected of having colorectal cancer) is analysed by 2D electrophoresis for detection of one or more of the CRCMPs as defined in Tables 1 and 2. A decreased or increased abundance of said one or more CRCMPs in the serum from the subject relative to serum from a subject or subjects free from colorectal cancer (e.g., a control sample) or a previously determined reference range indicates the presence or absence of colorectal cancer. More details are provided below in the section entitled Assay Measurement Strategies.

In a preferred embodiment, serum from a subject is analysed for quantitative detection of clusters of CRCMPs as defined in Tables 1 and 2.

As will be evident to one of skill in the art, a given CRCMP can be described according to the data provided for that CRCMP in Table 1 and in Table 2. The CRCMP is a protein comprising a peptide sequence described for that CRCMP (preferably comprising a plurality of, more preferably all of, the peptide sequences described for that CRCMP).

In one embodiment, serum from a subject is analysed for quantitative detection of one or more of the CRCMPs as defined in Tables 1 and 2, wherein a change in abundance of the CRCMP or CRCMPs in the serum from the subject relative to serum from a subject or subjects free from colorectal cancer (e.g., a control sample or a previously determined reference range) indicates the presence of colorectal cancer.

In a preferred embodiment, serum from a subject is analysed for quantitative detection of a cluster of CRCMPs as defined in Tables 1 and 2.

For each CRCMP the present invention additionally provides: (a) a preparation comprising the isolated CRCMP; (b) a preparation comprising one or more fragments of the CRCMP; and (c) antibodies or other affinity reagents such as Affibodies, Nanobodies or Unibodies that bind to said CRCMP, to said fragments, or both to said CRCMP and to said fragments. As used herein, a CRCMP is “isolated” when it is present in a preparation that is substantially free of contaminating proteins, i.e., a preparation in which less than 10% (preferably less than 5%, more preferably less than 1%) of the total protein present is contaminating protein(s). A contaminating protein is a protein having a significantly different amino acid sequence from that of the isolated CRCMP, as determined by mass spectral analysis. As used herein, a “significantly different” sequence is one that permits the contaminating protein to be resolved from the CRCMP by mass spectral analysis, performed according to the Reference Protocol.

The CRCMPs of the invention can be assayed by any method known to those skilled in the art, including but not limited to, the technology described herein in the examples, kinase assays, enzyme assays, binding assays and other functional assays, immunoassays, and western blotting. In one embodiment, the CRCMPs are separated on a 1-D gel by virtue of their MWs and visualized by staining the gel. In one embodiment, the CRCMPs are stained with a fluorescent dye and imaged with a fluorescence scanner. Sypro Red (Molecular Probes, Inc., Eugene, Oreg.) is a suitable dye for this purpose. A preferred fluorescent dye is disclosed in U.S. application Ser. No. 09/412,168, filed on Oct. 5, 1999, which is incorporated herein by reference in its entirety.

Alternatively, CRCMPs can be detected in an immunoassay. In one embodiment, an immunoassay is performed by contacting a sample from a subject to be tested with an anti-CRCMP antibody (or other affinity reagent such as an Affibody, Nanobody or Unibody) under conditions such that immunospecific binding can occur if the CRCMP is present, and detecting or measuring the amount of any immunospecific binding by the affinity reagent. Anti-CRCMP affinity reagents can be produced by the methods and techniques taught herein.

CRCMPs may be detected by virtue of the detection of a fragment thereof e.g. an immunogenic or antigenic fragment thereof. Fragments may have a length of at least 10, more typically at least 20 amino acids e.g. at least 50 or 100 amino acids e.g. at least 200 or 500 amino acids e.g at least 800 or 1000 amino acids.

In one embodiment, binding of antibody (or other affinity reagent such as an Affibody, Nanobody or Unibody) in tissue sections can be used to detect aberrant CRCMP localization or an aberrant level of one or more CRCMPs. In a specific embodiment, an antibody (or other affinity reagent such as an Affibody, Nanobody or Unibody) to a CRCMP can be used to assay a patient tissue (e.g., a serum sample) for the level of the CRCMP where an aberrant level of CRCMP is indicative of colorectal cancer. As used herein, an “aberrant level” means a level that is increased or decreased compared with the level in a subject free from colorectal cancer or a reference level.

Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, a CRCMP can be detected in a fluid sample (e.g., blood, urine, or saliva) by means of a two-step sandwich assay. In the first step, a capture reagent (e.g., an anti-CRCMP antibody or other affinity reagent such as an Affibody, Nanobody or Unibody) is used to capture the CRCMP. The capture reagent can optionally be immobilized on a solid phase. In the second step, a directly or indirectly labeled detection reagent is used to detect the captured CRCMP. In one embodiment, the detection reagent is a lectin. Any lectin can be used for this purpose that preferentially binds to the CRCMP rather than to other isoforms that have the same core protein as the CRCMP or to other proteins that share the antigenic determinant recognized by the affinity reagent. In a preferred embodiment, the chosen lectin binds to the CRCMP with at least 2-fold greater affinity, more preferably at least 5-fold greater affinity, still more preferably at least 10-fold greater affinity, than to said other isoforms that have the same core protein as the CRCMP or to said other proteins that share the antigenic determinant recognized by the affinity reagent. Based on the present description, a lectin that is suitable for detecting a given CRCMP can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174 (which is incorporated herein by reference in its entirety). In an alternative embodiment, the detection reagent is an antibody (or other affinity reagent such as an Affibody, Nanobody or Unibody), e.g., an antibody that immunospecifically detects other post-translational modifications, such as an antibody that immunospecifically binds to phosphorylated amino acids. Examples of such antibodies include those that bind to phosphotyrosine (BD Transduction Laboratories, catalog nos.: P11230-050/P11230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalogue nos. 71-8200, 13-9200).

If desired, a gene encoding a CRCMP, a related gene, or related nucleic acid sequences or subsequences, including complementary sequences, can also be used in hybridization assays. A nucleotide encoding a CRCMP, or subsequences thereof comprising at least 8 nucleotides, preferably at least 12 nucleotides, and most preferably at least 15 nucleotides can be used as a hybridization probe. Hybridization assays can be used for detection, prognosis, diagnosis, or monitoring of conditions, disorders, or disease states, associated with aberrant expression of genes encoding CRCMPs, or for differential diagnosis of subjects with signs or symptoms suggestive of colorectal cancer. In particular, such a hybridization assay can be carried out by a method comprising contacting a subject's sample containing nucleic acid with a nucleic acid probe capable of hybridizing to a DNA or RNA that encodes a CRCMP, under conditions such that hybridization can occur, and detecting or measuring any resulting hybridization. Nucleotides can be used for therapy of subjects having colorectal cancer, as described below.

The invention also provides kits e.g. diagnostic kits comprising one or more reagents for use in the detection and/or determination of one or more soluble polypeptide markers according to the invention. Suitably such kits comprise an anti-CRCMP antibody (or other affinity reagent such as an Affibody, Nanobody or Unibody) i.e. an affinity reagent capable of immunospecific binding to a soluble polypeptide marker according to the invention or for example a plurality of distinct such affinity reagents. Conveniently labeled affinity reagents may be employed to determine the presence of one or more of said soluble polypeptide markers. For example a kit may contain one or more containers with one or more affinity reagents against one or more said soluble polypeptide markers. Conveniently, such a kit may further comprise a labeled binding partner to the or each affinity reagent and/or a solid phase (such as a reagent strip) upon which the or each affinity reagent is immobilized. In addition, such a kit may optionally comprise one or more of the following: (1) instructions for using the anti-CRCMP affinity reagent for diagnosis, prognosis, therapeutic monitoring or any combination of these applications; (2) a labeled binding partner to the affinity reagent; (3) a solid phase (such as a reagent strip) upon which the anti-CRCMP affinity reagent is immobilized; and (4) a label or insert indicating regulatory approval for diagnostic, prognostic or therapeutic use or any combination thereof. If no labeled binding partner to the affinity reagent is provided, the anti-CRCMP affinity reagent itself can be labeled with a detectable marker, e.g., a chemiluminescent, enzymatic, fluorescent, or radioactive moiety.

Antibodies (or other affinity reagents such as Affibodies, Nanobodies or Unibodies) and kits may be used for diagnosing colorectal cancer in a subject, differentiating causes of colorectal cancer in a subject, guiding therapy in a subject suffering from colorectal cancer, assessing the risk of relapse in a subject suffering from colorectal cancer, or assigning a prognostic risk of one or more future clinical outcomes to a subject suffering from colorectal cancer.

Kits may also be of use in the detection, diagnosis of colorectal cancer in a subject, for differentiating causes of colorectal cancer in a subject, for guiding therapy in a subject suffering from colorectal cancer, for assessing the risk of relapse in a subject suffering from colorectal cancer, or for assigning a prognostic risk of one or more future clinical outcomes to a subject suffering from colorectal cancer, which kit comprises one or more soluble polypeptides derived from a protein selected from the list consisting of proteins defined by SEQ ID Nos 1-18, and/or one or more antigenic or immunogenic fragments thereof.

The invention also provides a kit comprising a nucleic acid probe capable of hybridizing to RNA encoding a CRCMP. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding a CRCMP, such as by polymerase chain reaction (see, e.g., Innis et al., 1990, PCR Protocols, Academic Press, Inc., San Diego, Calif.), ligase chain reaction (see EP 320,308) use of Qβ replicase, cyclic probe reaction, or other methods known in the art.

Kits are also provided which allow for the detection of a plurality of CRCMPs or a plurality of nucleic acids each encoding a CRCMP. A kit can optionally further comprise a predetermined amount of an isolated CRCMP protein or a nucleic acid encoding a CRCMP, e.g., for use as a standard or control.

### Use in Clinical Studies

The diagnostic methods and compositions of the present invention can assist in monitoring a clinical study, e.g. to evaluate drugs for therapy of colorectal cancer. In one embodiment, candidate molecules are tested for their ability to restore CRCMP levels in a subject having colorectal cancer to levels found in subjects free from colorectal cancer or, in a treated subject (e.g. after treatment with taxol or doxorubacin), to preserve CRCMP levels at or near non-colorectal cancer values. The levels of one or more CRCMPs can be assayed.

In another embodiment, the methods and compositions of the present invention are used to screen candidates for a clinical study to identify individuals having colorectal cancer; such individuals can then be excluded from the study or can be placed in a separate cohort for treatment or analysis. If desired, the candidates can concurrently be screened to identify individuals with colorectal cancer; procedures for these screens are well known in the art.

### Production of Proteins of the Invention and Corresponding Nucleic Acids

A DNA of the present invention can be obtained by isolation as a cDNA fragment from cDNA libraries using as starter materials commercial mRNAs and determining and identifying the nucleotide sequences thereof. That is, specifically, clones are randomly isolated from cDNA libraries, which are prepared according to Ohara et al's method (DNA Research Vol. 4, 53-59 (1997)). Next, through hybridization, duplicated clones (which appear repeatedly) are removed and then in vitro transcription and translation are carried out. Nucleotide sequences of both termini of clones, for which products of 50 kDa or more are confirmed, are determined.

Furthermore, databases of known genes are searched for homology using the thus obtained terminal nucleotide sequences as queries. The entire nucleotide sequence of a clone revealed to be novel as a result is determined. In addition to the above screening method, the 5′ and 3′ terminal sequences of cDNA are related to a human genome sequence. Then an unknown long-chain gene is confirmed in a region between the sequences, and the full-length of the cDNA is analyzed. In this way, an unknown gene that is unable to be obtained by a conventional cloning method that depends on known genes can be systematically cloned.

Moreover, all of the regions of a human-derived gene containing a DNA of the present invention can also be prepared using a PCR method such as RACE while paying sufficient attention to prevent artificial errors from taking place in short fragments or obtained sequences. As described above, clones having DNA of the present invention can be obtained.

In another means for cloning DNA of the present invention, a synthetic DNA primer having an appropriate nucleotide sequence of a portion of a polypeptide of the present invention is produced, followed by amplification by the PCR method using an appropriate library. Alternatively, selection can be carried out by hybridization of a DNA of the present invention with a DNA that has been incorporated into an appropriate vector and labeled with a DNA fragment or a synthetic DNA encoding some or all of the regions of a polypeptide of the present invention. Hybridization can be carried out by, for example, the method described in Current Protocols in Molecular Biology (edited by Frederick M. Ausubel et al., 1987). DNA of the present invention may be any DNA, as long as they contain nucleotide sequences encoding the polypeptides of the present invention as described above. Such a DNA may be a cDNA identified and isolated from cDNA libraries or the like that are derived from colorectal tissue. Such a DNA may also be a synthetic DNA or the like. Vectors for use in library construction may be any of bacteriophages, plasmids, cosmids, phargemids, or the like. Furthermore, by the use of a total RNA fraction or a mRNA fraction prepared from the above cells and/or tissues, amplification can be carried out by a direct reverse transcription coupled polymerase chain reaction (hereinafter abbreviated as “RT-PCR method”).

DNA encoding the above polypeptides consisting of amino acid sequences that are substantially identical to the amino acid sequences of the CRCMPs or DNA encoding the above polypeptides consisting of amino acid sequences derived from the amino acid sequences of the CRCMPs by deletion, substitution, or addition of one or more amino acids composing a portion of the amino acid sequence can be easily produced by an appropriate combination of, for example, a site-directed mutagenesis method, a gene homologous recombination method, a primer elongation method, and the PCR method known by persons skilled in the art. In addition, at this time, a possible method for causing a polypeptide to have substantially equivalent biological activity is substitution of homologous amino acids (e.g. polar and nonpolar amino acids, hydrophobic and hydrophilic amino acids, positively-charged and negatively charged amino acids, and aromatic amino acids) among amino acids composing the polypeptide. Furthermore, to maintain substantially equivalent biological activity, amino acids within functional domains contained in the polypeptide of the present invention are preferably conserved.

Furthermore, examples of DNA of the present invention include DNA comprising nucleotide sequences that encode the amino acid sequences of the CRCMPs and DNA hybridizing under stringent conditions to the DNA and encoding polypeptides (proteins) having biological activity (function) equivalent to the function of the polypeptides consisting of the amino acid sequences of the CRCMPs. Under such conditions, an example of such DNA capable of hybridizing to DNA comprising the nucleotide sequences that encode the amino acid sequences of the CRCMPs is DNA comprising a nucleotide sequence that has a degree of overall mean homology with the entire nucleotide sequence of the DNA, such as approximately 80% or more, preferably approximately 90% or more, and more preferably approximately 95% or more. Hybridization can be carried out according to a method known in the art such as a method described in Current Protocols in Molecular Biology (edited by Frederick M. Ausubel et al., 1987) or a method according thereto. Here, “stringent conditions” are, for example, conditions of approximately “1 *SSC, 0.1% SDS, and 37° C., more stringent conditions of approximately “0.5 *SSC, 0.1% SDS, and 42° C., or even more stringent conditions of approximately “0.2*SSC, 0.1% SDS, and 65° C. With more stringent hybridization conditions, the isolation of a DNA having high homology with a probe sequence can be expected. The above combinations of SSC, SDS, and temperature conditions are given for illustrative purposes. Stringency similar to the above can be achieved by persons skilled in the art using an appropriate combination of the above factors or other factors (for example, probe concentration, probe length, and reaction time for hybridization) for determination of hybridization stringency.

A cloned DNA of the present invention can be directly used or used, if desired, after digestion with a restriction enzyme or addition of a linker, depending on purposes. The DNA may have ATG as a translation initiation codon at the 5′ terminal side and have TAA, TGA, or TAG as a translation termination codon at the 3′ terminal side. These translation initiation and translation termination codons can also be added using an appropriate synthetic DNA adapter.

Where they are provided for use with the methods of the invention the CRCMPs are preferably provided in isolated form. More preferably the CRCMP polypeptides have been purified to at least to some extent. The CRCMP polypeptides may be provided in substantially pure form, that is to say free, to a substantial extent, from other proteins. The CRCMP polypeptides can also be produced using recombinant methods, synthetically produced or produced by a combination of these methods. The CRCMPs can be easily prepared by any method known by persons skilled in the art, which involves producing an expression vector containing a DNA of the present invention or a gene containing a DNA of the present invention, culturing a transformant transformed using the expression vector, generating and accumulating a polypeptide of the present invention or a recombinant protein containing the polypeptide, and then collecting the resultant.

Recombinant CRCMP polypeptides may be prepared by processes well known in the art from genetically engineered host cells comprising expression systems. Accordingly, the present invention also relates to expression systems which comprise CRCMP polypeptides or nucleic acids, to host cells which are genetically engineered with such expression systems and to the production of CRCMP polypeptides by recombinant techniques. For recombinant CRCMP polypeptide production, host cells can be genetically engineered to incorporate expression systems or portions thereof for nucleic acids. Such incorporation can be performed using methods well known in the art, such as, calcium phosphate transfection, DEAD-dextran mediated transfection, transvection, microinjection, cationic lipid-mediated transfection, electroporation, transduction, scrape loading, ballistic introduction or infection (see e.g. Davis et al., Basic Methods in Molecular Biology, 1986 and Sambrook et al., Molecular Cloning: A Laboratory Manual, 2nd Ed., Cold Spring Harbour laboratory Press, Cold Spring Harbour, N.Y., 1989).

As host cells, for example, bacteria of the genus Escherichia, Streptococci, Staphylococci, Streptomyces, bacteria of the genus Bacillus, yeast, Aspergillus cells, insect cells, insects, and animal cells are used. Specific examples of bacteria of the genus Escherichia, which are used herein, include Escherichia coli K12 and DH1 (Proc. Natl. Acad. Sci. U.S.A., Vol. 60, 160 (1968)), JM103 (Nucleic Acids Research, Vol. 9, 309 (1981)), JA221 (Journal of Molecular Biology, Vol. 120, 517 (1978)), and HB101 (Journal of Molecular Biology, Vol. 41, 459 (1969)). As bacteria of the genus Bacillus, for example, Bacillus subtilis MI114 (Gene, Vol. 24, 255 (1983)) and 207-21 (Journal of Biochemistry, Vol. 95, 87 (1984)) are used. As yeast, for example, Saccaromyces cerevisiae AH22, AH22R-, NA87-11A, DKD-5D, and 20B-12, Schizosaccaromyces pombe NCYC1913 and NCYC2036, and Pichia pastoris are used. As insect cells, for example, Drosophila S2 and Spodoptera Sf9 cells are used. As animal cells, for example, COS-7 and Vero monkey cells, CHO Chinese hamster cells (hereinafter abbreviated as CHO cells), dhfr-gene-deficient CHO cells, mouse L cells, mouse AtT-20 cells, mouse myeloma cells, rat GH3 cells, human FL cells, COS, HeLa, C127, 3T3, HEK 293, BHK and Bowes melanoma cells are used.

Cell-free translation systems can also be employed to produce recombinant polypeptides (e.g. rabbit reticulocyte lysate, wheat germ lysate, SP6/T7 in vitro T&T and RTS 100 E. Coli HY transcription and translation kits from Roche Diagnostics Ltd., Lewes, UK and the TNT Quick coupled Transcription/Translation System from Promega UK, Southampton, UK).

The expression vector can be produced according to a method known in the art. For example, the vector can be produced by (1) excising a DNA fragment containing a DNA of the present invention or a gene containing a DNA of the present invention and (2) ligating the DNA fragment downstream of the promoter in an appropriate expression vector. A wide variety of expression systems can be used, such as and without limitation, chromosomal, episomal and virus-derived systems, e.g. plasmids derived from Escherichia coli (e.g. pBR322, pBR325, pUC18, and pUC118), plasmids derived from Bacillus subtilis (e.g. pUB110, pTP5, and pC194), from bacteriophage, from transposons, from yeast episomes (e.g. pSH19 and pSH15), from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage (such as [lambda] phage) genetic elements, such as cosmids and phagemids. The expression systems may contain control regions that regulate as well as engender expression. Promoters to be used in the present invention may be any promoters as long as they are appropriate for hosts to be used for gene expression. For example, when a host is Escherichia coli, a trp promoter, a lac promoter, a recA promoter, a pL promoter, an lpp promoter, and the like are preferred. When a host is Bacillus subtilis, an SPO1 promoter, an SPO2 promoter, a penP promoter, and the like are preferred. When a host is yeast, a PHO5 promoter, a PGK promoter, a GAP promoter, an ADH promoter, and the like are preferred. When an animal cell is used as a host, examples of promoters for use in this case include an SRa promoter, an SV40 promoter, an LTR promoter, a CMV promoter, and an HSV-TK promoter. Generally, any system or vector that is able to maintain, propagate or express a nucleic acid to produce a polypeptide in a host may be used.

The appropriate nucleic acid sequence may be inserted into an expression system by any variety of well known and routine techniques, such as those set forth in Sambrook et al., supra. Appropriate secretion signals may be incorporated into the CRCMP polypeptide to allow secretion of the translated protein into the lumen of the endoplasmic reticulum, the periplasmic space or the extracellular environment. These signals may be endogenous to the CRCMP polypeptide or they may be heterologous signals. Transformation of the host cells can be carried out according to methods known in the art. For example, the following documents can be referred to: Proc. Natl. Acad. Sci. U.S.A., Vol. 69, 2110 (1972); Gene, Vol. 17, 107 (1982); Molecular & General Genetics, Vol. 168, 111 (1979); Methods in Enzymology, Vol. 194, 182-187 (1991); Proc. Natl. Acad. Sci. U.S.A.), Vol. 75, 1929 (1978); Cell Technology, separate volume 8, New Cell Technology, Experimental Protocol. 263-267 (1995) (issued by Shujunsha); and Virology, Vol. 52, 456 (1973). The thus obtained transformant transformed with an expression vector containing a DNA of the present invention or a gene containing a DNA of the present invention can be cultured according to a method known in the art. For example, when hosts are bacteria of the genus Escherichia, the bacteria are generally cultured at approximately 15° C. to 43° C. for approximately 3 to 24 hours. If necessary, aeration or agitation can also be added. When hosts are bacteria of the genus Bacillus, the bacteria are generally cultured at approximately 30° C. to 40° C. for approximately 6 to 24 hours. If necessary, aeration or agitation can also be added. When transformants whose hosts are yeast are cultured, culture is generally carried out at approximately 20° C. to 35° C. for approximately 24 to 72 hours using media with pH adjusted to be approximately 5 to 8. If necessary, aeration or agitation can also be added. When transformants whose hosts are animal cells are cultured, the cells are generally cultured at approximately 30° C. to 40° C. for approximately 15 to 60 hours using media with the pH adjusted to be approximately 6 to 8. If necessary, aeration or agitation can also be added.

If a CRCMP polypeptide is to be expressed for use in cell-based screening assays, it is preferred that the polypeptide be produced at the cell surface. In this event, the cells may be harvested prior to use in the screening assay. If the CRCMP polypeptide is secreted into the medium, the medium can be recovered in order to isolate said polypeptide. If produced intracellularly, the cells must first be lysed before the CRCMP polypeptide is recovered.

CRCMP polypeptides can be recovered and purified from recombinant cell cultures or from other biological sources by well known methods including, ammonium sulphate or ethanol precipitation, acid extraction, anion or cation exchange chromatography, phosphocellulose chromatography, affinity chromatography, hydrophobic interaction chromatography, hydroxylapatite chromatography, molecular sieving chromatography, centrifugation methods, electrophoresis methods and lectin chromatography. In one embodiment, a combination of these methods is used. In another embodiment, high performance liquid chromatography is used. In a further embodiment, an antibody (or other affinity reagent such as an Affibody, Nanobody or Unibody) which specifically binds to a CRCMP polypeptide can be used to deplete a sample comprising a CRCMP polypeptide of said polypeptide or to purify said polypeptide.

To separate and purify a polypeptide or a protein of the present invention from the culture products, for example, after culture, microbial bodies or cells are collected by a known method, they are suspended in an appropriate buffer, the microbial bodies or the cells are disrupted by, for example, ultrasonic waves, lysozymes, and/or freeze-thawing, the resultant is then subjected to centrifugation or filtration, and then a crude extract of the protein can be obtained. The buffer may also contain a protein denaturation agent such as urea or guanidine hydrochloride or a surfactant such as Triton X-100™. When the protein is secreted in a culture solution, microbial bodies or cells and a supernatant are separated by a known method after the completion of culture and then the supernatant is collected. The protein contained in the thus obtained culture supernatant or the extract can be purified by an appropriate combination of known separation and purification methods. The thus obtained polypeptides (proteins) of the present invention can be converted into salts by a known method or a method according thereto. Conversely, when the polypeptides (proteins) of the present invention are obtained in the form of salts, they can be converted into free proteins or peptides or other salts by a known method or a method according thereto. Moreover, an appropriate protein modification enzyme such as trypsin or chymotrypsin is caused to act on a protein produced by a recombinant before or after purification, so that modification can be arbitrarily added or a polypeptide can be partially removed. The presence of polypeptides (proteins) of the present invention or salts thereof can be measured by various binding assays, enzyme immunoassays using specific antibodies, and the like.

Techniques well known in the art may be used for refolding to regenerate native or active conformations of the CRCMP polypeptides when the polypeptides have been denatured during isolation and or purification. In the context of the present invention, CRCMP polypeptides can be obtained from a biological sample from any source, such as and without limitation, a blood sample or tissue sample, e.g. a colorectal tissue sample.

CRCMP polypeptides may be in the form of “mature proteins” or may be part of larger proteins such as fusion proteins. It is often advantageous to include an additional amino acid sequence which contains secretory or leader sequences, a pre-, pro- or prepro-protein sequence, or a sequence which aids in purification such as an affinity tag, for example, but without limitation, multiple histidine residues, a FLAG tag, HA tag or myc tag.

An additional sequence that may provide stability during recombinant production may also be used. Such sequences may be optionally removed as required by incorporating a cleavable sequence as an additional sequence or part thereof. Thus, a CRCMP polypeptide may be fused to other moieties including other polypeptides or proteins (for example, glutathione S-transferase and protein A). Such a fusion protein can be cleaved using an appropriate protease, and then separated into each protein. Such additional sequences and affinity tags are well known in the art. In addition to the above, features known in the art, such as an enhancer, a splicing signal, a polyA addition signal, a selection marker, and an SV40 replication origin can be added to an expression vector, if desired.

### Diagnosis of Colorectal Cancer

In accordance with the present invention, test samples of serum, plasma or urine obtained from a subject suspected of having or known to have colorectal cancer can be used for diagnosis or monitoring. In one embodiment, a change in the abundance of one or more CRCMPs in a test sample relative to a control sample (from a subject or subjects free from colorectal cancer) or a previously determined reference range indicates the presence of colorectal cancer; CRCMPs suitable for this purpose are defined in Tables 1 and 2, as described in detail above. In another embodiment, the relative abundance of one or more CRCMPs in a test sample compared to a control sample or a previously determined reference range indicates a subtype of colorectal cancer (e.g., familial or sporadic colorectal cancer). In yet another embodiment, the relative abundance of one or more CRCMPs in a test sample relative to a control sample or a previously determined reference range indicates the degree or severity of colorectal cancer (e.g., the likelihood for metastasis). In any of the aforesaid methods, detection of one or more CRCMPs as defined in Tables 1 and 2 herein may optionally be combined with detection of one or more additional biomarkers for colorectal cancer. Any suitable method in the art can be employed to measure the level of CRCMPs, including but not limited to the technology described herein in the examples, kinase assays, immunoassays to detect and/or visualize the CRCMPs (e.g., Western blot, immunoprecipitation followed by sodium dodecyl sulfate polyacrylamide gel electrophoresis, immunocytochemistry, etc.). In cases where a CRCMP has a known function, an assay for that function may be used to measure CRCMP expression. In a further embodiment, a change in the abundance of mRNA encoding one or more CRCMPs as defined in Tables 1 and 2 in a test sample relative to a control sample or a previously determined reference range indicates the presence of colorectal cancer. Any suitable hybridization assay can be used to detect CRCMP expression by detecting and/or visualizing mRNA encoding the CRCMP (e.g., Northern assays, dot blots, in situ hybridization, etc.).

In another embodiment of the invention, labeled antibodies (or other affinity reagents such as Affibodies, Nanobodies or Unibodies), derivatives and analogs thereof, which specifically bind to a CRCMP can be used for diagnostic purposes to detect, diagnose, or monitor colorectal cancer. Preferably, colorectal cancer is detected in an animal, more preferably in a mammal and most preferably in a human.

### Assay Measurement Strategies

Preferred assays are “configured to detect” a particular marker. That an assay is “configured to detect” a marker means that an assay can generate a detectable signal indicative of the presence or amount of a physiologically relevant concentration of a particular marker of interest. Such an assay may, but need not, specifically detect a particular marker (i.e., detect a marker but not some or all related markers). Because an antibody epitope is on the order of 8 amino acids, an immunoassay will detect other polypeptides (e.g., related markers) so long as the other polypeptides contain the epitope(s) necessary to bind to the antibody used in the assay. Such other polypeptides are referred to as being “immunologically detectable” in the assay, and would include various isoforms (e.g., splice variants). In the case of a sandwich immunoassay, related markers must contain at least the two epitopes bound by the antibody used in the assay in order to be detected. Taking BNP₇₉₋₁₀₈ as an example, an assay configured to detect this marker may also detect BNP₇₇₋₁₀₈ or BNP₁₋₁₀₈, as such molecules may also contain the epitope(s) present on BNP₇₉₋₁₀₈ to which the assay antibody binds. However, such assays may also be configured to be “sensitive” to loss of a particular epitope, e.g., at the amino and/or carboxyl terminus of a particular polypeptide of interest as described in US2005/0148024, which is hereby incorporated by reference in its entirety. As described therein, an antibody may be selected that would bind to the amino terminus of BNP₇₉₋₁₀₈ such that it does not bind to BNP₇₇₋₁₀₈. Similar assays that bind BNP₃₋₁₀₈ and that are “sensitive” to loss of a particular epitope, e.g., at the amino and/or carboxyl terminus are also described therein.

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

Preferably the markers are analyzed using an immunoassay, and most preferably sandwich immunoassay, although other methods are well known to those skilled in the art (for example, the measurement of marker RNA levels). The presence or amount of a marker is generally determined using antibodies (or other affinity reagents such as Affibodies, Nanobodies or Unibodies) specific for each marker and detecting specific binding. Any suitable immunoassay may be utilized, for example, enzyme-linked immunoassays (ELISA), radioimmunoassays (RIAs), competitive binding assays, and the like. Specific immunological binding of the affinity reagent to the marker can be detected directly or indirectly. Direct labels include fluorescent or luminescent tags, metals, dyes, radionuclides, and the like, attached to the affinity reagent. Indirect labels include various enzymes well known in the art, such as alkaline phosphatase, horseradish peroxidase and the like.

The use of immobilized antibodies (or other affinity reagents such as Affibodies, Nanobodies or Unibodies) specific for the markers is also contemplated by the present invention. The affinity reagents could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the affinity reagent or a plurality of affinity reagents in an array on solid support. This strip could then be dipped into the test sample and then processed quickly through washes and detection steps to generate a measurable signal, such as a colored spot.

For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses perform simultaneous assays of a plurality of markers using a single test device. Particularly useful physical formats comprise surfaces having a plurality of discrete, addressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Preferred assay devices of the present invention will comprise, for one or more assays, a first antibody (or other affinity reagent such as an Affibody, Nanobody or Unibody) conjugated to a solid phase and a second antibody (or other affinity reagent such as an Affibody, Nanobody or Unibody) conjugated to a signal development element. Such assay devices are configured to perform a sandwich immunoassay for one or more analytes. These assay devices will preferably further comprise a sample application zone, and a flow path from the sample application zone to a second device region comprising the first antibody (or other affinity reagent such as an Affibody, Nanobody or Unibody) conjugated to a solid phase.

Flow of a sample along the flow path may be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Most preferably, sample applied to the sample application zone will contact both a first antibody (or other affinity reagent such as an Affibody, Nanobody or Unibody) conjugated to a solid phase and a second antibody (or other affinity reagent such as an Affibody, Nanobody or Unibody) conjugated to a signal development element along the flow path (sandwich assay format). Additional elements, such as filters to separate plasma or serum from blood, mixing chambers, etc., may be included as required by the artisan. Exemplary devices are described in Chapter 41, entitled “Near Patient Tests Triage® Cardiac System,” in The Immunoassay Handbook, 2^{nd} ed., David Wild, ed., Nature Publishing Group, 2001, which is hereby incorporated by reference in its entirety.

A panel consisting of the markers referenced above may be constructed to provide relevant information related to differential diagnosis. Such a panel may be constructed using 1, 2, 3, 4, 5, 6, 7, 8, 9, 10, 12, 15, 20 or more individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W.B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of markers. Such a kit preferably comprises devices and reagents for the analysis of at least one test sample and instructions for performing the assay. Optionally the kits may contain one or more means for using information obtained from immunoassays performed for a marker panel to rule in or out certain diagnoses. Other measurement strategies applicable to the methods described herein include chromatography (e.g., HPLC), mass spectrometry, receptor-based assays, and combinations of the foregoing.

### Production of Affinity Reagents to the CRCMPs

According to those in the art, there are three main types of affinity reagent-monoclonal antibodies, phage display antibodies and small molecules such as Affibodies, Domain Antibodies (dAbs), Nanobodies or Unibodies. In general in applications according to the present invention where the use of antibodies is stated, other affinity reagents (e.g. Affibodies, domain antibodies, Nanobodies or Unibodies) may be employed.

### Production of Antibodies to the CRCMPs

According to the invention a CRCMP, a CRCMP analog, a CRCMP-related protein or a fragment or derivative of any of the foregoing may be used as an immunogen to generate antibodies which immunospecifically bind such an immunogen. Such immunogens can be isolated by any convenient means, including the methods described above. The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody”. Antibodies of the invention include, but are not limited to polyclonal, monoclonal, bispecific, humanized or chimeric antibodies, single chain antibodies, Fab fragments and F(ab′)₂ fragments, fragments produced by a Fab expression library, anti-idiotypic (anti-Id) antibodies, and epitope-binding fragments of any of the above. The immunoglobulin molecules of the invention can be of any class (e.g. IgG, IgE, IgM, IgD and IgA) or subclass of immunoglobulin molecule.

The term “specifically binds” (or “immunospecifically binds”) is not intended to indicate that an antibody binds exclusively to its intended target. Rather, an antibody “specifically binds” if its affinity for its intended target is about 5-fold greater when compared to its affinity for a non-target molecule. Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about M⁻¹.

Affinity is calculated as K_{d}=k_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(n−r):

where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. No. 6,316,409). The affinity of a targeting agent for its target molecule is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

In one embodiment, antibodies that recognize gene products of genes encoding CRCMPs are publicly available. In another embodiment, methods known to those skilled in the art are used to produce antibodies that recognize a CRCMP, a CRCMP analog, a CRCMP-related polypeptide, or a fragment or derivative of any of the foregoing. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In one embodiment of the invention, antibodies to a specific domain of a CRCMP are produced. In a specific embodiment, hydrophilic fragments of a CRCMP are used as immunogens for antibody production.

In the production of antibodies, screening for the desired antibody can be accomplished by techniques known in the art, e.g. ELISA (enzyme-linked immunosorbent assay). For example, to select antibodies which recognize a specific domain of a CRCMP, one may assay generated hybridomas for a product which binds to a CRCMP fragment containing such domain. For selection of an antibody that specifically binds a first CRCMP homolog but which does not specifically bind to (or binds less avidly to) a second CRCMP homolog, one can select on the basis of positive binding to the first CRCMP homolog and a lack of binding to (or reduced binding to) the second CRCMP homolog. Similarly, for selection of an antibody that specifically binds a CRCMP but which does not specifically bind to (or binds less avidly to) a different isoform of the same protein (such as a different glycoform having the same core peptide as the CRCMP), one can select on the basis of positive binding to the CRCMP and a lack of binding to (or reduced binding to) the different isoform (e.g. a different glycoform). Thus, the present invention provides antibodies (preferably monoclonal antibodies) that bind with greater affinity (preferably at least 2-fold, more preferably at least 5-fold, still more preferably at least 10-fold greater affinity) to the CRCMPs than to a different isoform or isoforms (e.g. glycoforms) of the CRCMPs.

Polyclonal antibodies which may be used in the methods of the invention are heterogeneous populations of antibody molecules derived from the sera of immunized animals. Unfractionated immune serum can also be used. Various procedures known in the art may be used for the production of polyclonal antibodies to a CRCMP, a fragment of a CRCMP, a CRCMP-related polypeptide, or a fragment of a CRCMP-related polypeptide. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be used to immunize by injection various host animals, including but not limited to rabbits, mice, rats, etc., to generate polyclonal or monoclonal antibodies. The Preferred Technology described herein provides isolated CRCMPs suitable for such immunization. If a CRCMP is purified by gel electrophoresis, the CRCMP can be used for immunization with or without prior extraction from the polyacrylamide gel. Various adjuvants (i.e. immunostimulants) may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward a CRCMP, a fragment of a CRCMP, a CRCMP-related polypeptide, or a fragment of a CRCMP-related polypeptide, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g. human-mouse chimeras). A chimeric antibody is a molecule in which different portions are derived from different animal species, such as those having a human immunoglobulin constant region and a variable region derived from a murine mAb. (See, e.g. Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g. Queen, U.S. Pat. No. 5,585,089, which is incorporated herein by reference in its entirety.)

Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chain genes, but which can express human heavy and light chain genes. The transgenic mice are immunized in the normal fashion with a selected antigen, e.g. all or a portion of a CRCMP. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g. U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection”. In this approach a selected non-human monoclonal antibody, e.g. a mouse antibody, is used to guide the selection of a completely human antibody recognizing the same epitope. (Jespers et al. (1994) Bio/technology 12:899-903).

The antibodies of the present invention can also be generated by the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims. In particular, such phage can be utilized to display antigen binding domains expressed from a repertoire or combinatorial antibody library (e.g. human or murine). Phage expressing an antigen binding domain that binds the antigen of interest can be selected or identified with antigen, e.g. using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g. as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published 13 May 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al., Methods in Enzymology, 1986, 121:210.

The invention provides functionally active fragments, derivatives or analogs of the anti-CRCMP immunoglobulin molecules. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)₂ fragments and Fab fragments. Antibody fragments which recognize specific epitopes may be generated by known techniques. F(ab′)₂ fragments consist of the variable region, the light chain constant region and the CH1 domain of the heavy chain and are generated by pepsin digestion of the antibody molecule. Fab fragments are generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g. as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g. a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are modified, i.e., by the covalent attachment of any type of molecule as long as such covalent attachment does not impair immunospecific binding. For example, but not by way of limitation, the derivatives and analogs of the immunoglobulins include those that have been further modified, e.g. by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical amino acids.

The foregoing antibodies can be used in methods known in the art relating to the localization and activity of the CRCMPs, e.g. for imaging these proteins, measuring levels thereof in appropriate physiological samples, in diagnostic methods, etc.

### Production of Affibodies to the CRCMPs

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55.). The simple, robust structure of Affibody molecules in combination with their low molecular weight (6 kDa), make them suitable for a wide variety of applications, for instance, as detection reagents (Ronmark J, Hansson M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

Labelled Affibodies may also be useful in imaging applications for determining abundance of Isoforms.

### Production of Domain Antibodies to the CRCMPs

Domain Antibodies (dAbs) are the smallest functional binding units of antibodies, corresponding to the variable regions of either the heavy (V_{H}) or light (V_{L}) chains of human antibodies. Domain Antibodies have a molecular weight of approximately 13 kDa. Domantis has developed a series of large and highly functional libraries of fully human V_{H} and V_{L} dAbs (more than ten billion different sequences in each library), and uses these libraries to select dAbs that are specific to therapeutic targets. In contrast to many conventional antibodies, Domain Antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; US Serial No. 2004/0110941; European patent application No. 1433846 and European Patents 0368684 & 0616640; WO05/035572, WO04/101790, WO04/081026, WO04/058821, WO04/003019 and WO03/002609, each of which is herein incorporated by reference in its entirety.

### Production of Nanobodies to the CRCMPs

Nanobodies are antibody-derived therapeutic proteins that contain the unique structural and functional properties of naturally-occurring heavy-chain antibodies. These heavy-chain antibodies contain a single variable domain (VHH) and two constant domains (C_{H}2 and C_{H}3). Importantly, the cloned and isolated VHH domain is a perfectly stable polypeptide harbouring the full antigen-binding capacity of the original heavy-chain antibody. Nanobodies have a high homology with the VH domains of human antibodies and can be further humanised without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see e.g. WO 04/041867, which is herein incorporated by reference in its entirety) and are easy to manufacture. Other advantages of Nanobodies include recognising uncommon or hidden epitopes as a result of their small size, bindings into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts e.g. E. coli (see e.g. U.S. Pat. No. 6,765,087 which is herein incorporated by reference in its entirety) moulds (for example Aspergillus or Trichoderma) and yeast (for example Saccharomyces, Kluyveromyces, Hansenula or Pichia) (see e.g. U.S. Pat. No. 6,838,254 which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see e.g. WO 06/079372, which is herein incorporated by reference in its entirety) is a proprietary method for generating Nanobodies against a desired target, based on automated high-throughout selection of B-cells.

### Production of Unibodies to the CRCMPs

UniBody is a new proprietary antibody technology that creates a stable, smaller antibody format with an anticipated longer therapeutic window than current small antibody formats. IgG4 antibodies are considered inert and thus do not interact with the immune system. Genmab modified fully human IgG4 antibodies by eliminating the hinge region of the antibody. Unlike the full size IgG4 antibody, the half molecule fragment is very stable and is termed a UniBody. Halving the IgG4 molecule left only one area on the UniBody that can bind to disease targets and the UniBody therefore binds univalently to only one site on target cells. This univalent binding does not stimulate cancer cells to grow like bivalent antibodies might and opens the door for treatment of some types of cancer which ordinary antibodies cannot treat.

The UniBody is about half the size of a regular IgG4 antibody. This small size can be a great benefit when treating some forms of cancer, allowing for better distribution of the molecule over larger solid tumors and potentially increasing efficacy.

Fabs typically do not have a very long half-life. UniBodies, however, were cleared at a similar rate to whole IgG4 antibodies and were able to bind as well as whole antibodies and antibody fragments in pre-clinical studies. Other antibodies primarily work by killing the targeted cells whereas UniBodies only inhibit or silence the cells.

### Expression of Affinity Reagents

### Expression of Antibodies

The antibodies of the invention can be produced by any method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g. as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g. an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g. as described in Huse et al., 1989, Science 246:1275-1281) for clones of Fab fragments that bind the specific antigen or by screening antibody libraries (See, e.g. Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g. PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g. humanized antibodies.

Once a nucleic acid encoding an antibody molecule of the invention has been obtained, the vector for the production of the antibody molecule may be produced by recombinant DNA technology using techniques well known in the art. Thus, methods for preparing the proteins of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2^{nd} Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus are an effective expression system for antibodies (Foecking et al., 1986, Gene 45:101; Cockett et al., 1990, Bio/Technology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g. E. coli, B. subtilis) transformed with recombinant bacteriophage DNA, plasmid DNA or cosmid DNA expression vectors containing antibody coding sequences; yeast (e.g. Saccharomyces, Pichia) transformed with recombinant yeast expression vectors containing antibody coding sequences; insect cell systems infected with recombinant virus expression vectors (e.g. baculovirus) containing the antibody coding sequences; plant cell systems infected with recombinant virus expression vectors (e.g. cauliflower mosaic virus, CaMV; tobacco mosaic virus, TMV) or transformed with recombinant plasmid expression vectors (e.g. Ti plasmid) containing antibody coding sequences; or mammalian cell systems (e.g. COS, CHO, BHK, 293, 3T3 cells) harboring recombinant expression constructs containing promoters derived from the genome of mammalian cells (e.g. metallothionein promoter) or from mammalian viruses (e.g. the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g. an adenovirus expression system) may be utilized.

As discussed above, a host cell strain may be chosen which modulates the expression of the inserted sequences, or modifies and processes the gene product in the specific fashion desired. Such modifications (e.g. glycosylation) and processing (e.g. cleavage) of protein products may be important for the function of the protein.

For long-term, high-yield production of recombinant antibodies, stable expression is preferred. For example, cell lines that stably express an antibody of interest can be produced by transfecting the cells with an expression vector comprising the nucleotide sequence of the antibody and the nucleotide sequence of a selectable (e.g. neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g. ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni²⁺ nitriloacetic acid-agarose columns and histidine-tagged proteins are selectively eluted with imidazole-containing buffers.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) is present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

For therapeutic applications, antibodies (particularly monoclonal antibodies) may suitably be human or humanized animal (e.g. mouse) antibodies. Animal antibodies may be raised in animals using the human protein (e.g. a CRCMP) as immunogen. Humanisation typically involves grafting CDRs identified thereby into human framework regions. Normally some subsequent retromutation to optimize the conformation of chains is required. Such processes are known to persons skilled in the art.

### Expression of Affibodies

The construction of affibodies has been described elsewhere (Ronnmark J, Gronlund H, Uhle'n, M., Nygren P.A°, Human immunoglobulin A (IgA)-specific ligands from combinatorial engineering of protein A, 2002, Eur. J. Biochem. 269, 2647-2655.), including the construction of affibody phage display libraries (Nord, K., Nilsson, J., Nilsson, B., Uhle'n, M. & Nygren, P.A°, A combinatorial library of an a-helical bacterial receptor domain, 1995, Protein Eng. 8, 601-608. Nord, K., Gunneriusson, E., Ringdahl, J., Sta°hl, S., Uhle'n, M. & Nygren, P.A°, Binding proteins selected from combinatorial libraries of an a-helical bacterial receptor domain, 1997, Nat. Biotechnol. 15, 772-777.)

The biosensor analyses to investigate the optimal affibody variants using biosensor binding studies has also been described elsewhere (Ronnmark J, Gronlund H, Uhle'n, M., Nygren P.A°, Human immunoglobulin A (IgA)-specific ligands from combinatorial engineering of protein A, 2002, Eur. J. Biochem. 269, 2647-2655.).

### Conjugated Affinity Reagents

In a preferred embodiment, anti-CRCMP affinity reagents such as antibodies or fragments thereof are conjugated to a diagnostic or therapeutic moiety. The antibodies can be used for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc. ⁶⁸Ga may also be employed.

Anti-CRCMP antibodies or fragments thereof can be conjugated to a therapeutic agent or drug moiety to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g. angiostatin or endostatin; or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Techniques for conjugating such therapeutic moiety to antibodies are well known, see, e.g. Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

### Identification of Marker Panels

In accordance with the present invention, there are provided methods and systems for the identification of one or more markers useful in diagnosis, prognosis, and/or determining an appropriate therapeutic course. One skilled in the art will also recognize that univariate analysis of markers can be performed and the data from the univariate analyses of multiple markers can be combined to form panels of markers to differentiate different disease conditions in a variety of ways, including so-called “n-of-m” methods (for example, where if n markers (e.g., 2) out of a total of m markers (e.g., 3) meet some criteria, the test is considered positive), multiple linear regression, determining interaction terms, stepwise regression, etc.

Suitable methods for identifying markers useful for such purposes are also described in detail in U.S. Provisional Patent Application No. 60/436,392 filed Dec. 24, 2002, PCT application US03/41426 filed Dec. 23, 2003, U.S. patent application Ser. No. 10/331,127 filed Dec. 27, 2002, and PCT application No. US03/41453, each of which is hereby incorporated by reference in its entirety, including all tables, figures, and claims. The following discussion provides an exemplary discussion of methods that may be used to provide the panels of the present invention.

In developing a panel of markers, data for a number of potential markers may be obtained from a group of subjects by testing for the presence or level of certain markers. The group of subjects is divided into two sets. The first set includes subjects who have been confirmed as having a disease, outcome, or, more generally, being in a first condition state. For example, this first set of patients may be those diagnosed with colorectal cancer that died as a result of that disease. Hereinafter, subjects in this first set will be referred to as “diseased.”

The second set of subjects is simply those who do not fall within the first set. Subjects in this second set will hereinafter be referred to as “non-diseased”. Preferably, the first set and the second set each have an approximately equal number of subjects. This set may be normal patients, and/or patients suffering from another cause of colorectal cancer, and/or patients that lived to a particular endpoint of interest.

The data obtained from subjects in these sets preferably includes levels of a plurality of markers. Preferably, data for the same set of markers is available for each patient. This set of markers may include all candidate markers that may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a single marker often is incapable of definitively identifying a subject as falling within a first or second group in a prospective fashion. For example, if a patient is measured as having a marker level that falls within an overlapping region in the distribution of diseased and non-diseased subjects, the results of the test may be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve. ROC curves are well known to those skilled in the art.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

As discussed above, the measurement of the level of a single marker may have limited usefulness, e.g., it may be non-specifically increased due to inflammation. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In a preferred method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. In a preferred embodiment, the panel response (R) for each subject (j) is expressed as:

Rⱼ=ΣwᵢI_{i,j},

where i is the marker index, j is the subject index, wᵢ is the weighting coefficient for marker i, I is the indicator value to which the marker level for marker i is mapped for subject j, and Σ is the summation over all candidate markers i. This panel response value may be referred to as a “panel index.”

One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. Certain markers may be then be changed or even eliminated from the panel, and the process repeated until a satisfactory result is obtained. The effective contribution of each marker in the panel may be determined to identify the relative importance of the markers. In one embodiment, the weighting coefficients resulting from the optimization process may be used to determine the relative importance of each marker. The markers with the lowest coefficients may be eliminated or replaced.

In certain cases, the lower weighting coefficients may not be indicative of a low importance. Similarly, a higher weighting coefficient may not be indicative of a high importance. For example, the optimization process may result in a high coefficient if the associated marker is irrelevant to the diagnosis. In this instance, there may not be any advantage that will drive the coefficient lower. Varying this coefficient may not affect the value of the objective function.

### Evaluation of Marker Panels

To allow a determination of test accuracy, a “gold standard” test criterion may be selected which allows selection of subjects into two or more groups for comparison by the foregoing methods. In the case of colorectal cancer, this gold standard may be the carcinoembyonic antigen (CEA) test. This implies that those negative for the gold standard are free of colorectal cancer. Alternatively, an initial comparison of confirmed colorectal cancer subjects may be compared to normal healthy control subjects. In the case of a prognosis, mortality is a common test criterion.

The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al., Radiology 143: 29-36 (1982).

Measures of test accuracy may be obtained as described in Fischer et al., Intensive Care Med. 29: 1043-51, 2003, and used to determine the effectiveness of a given marker or panel of markers. These measures include sensitivity and specificity, predictive values, likelihood ratios, diagnostic odds ratios, and ROC curve areas. As discussed above, preferred tests and assays exhibit one or more of the following results on these various measures:
at least 75% sensitivity, combined with at least 75% specificity;
ROC curve area of at least 0.6, more preferably 0.7, still more preferably at least 0.8, even more preferably at least 0.9, and most preferably at least 0.95; and/or
at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement; and/or
a positive likelihood ratio and/or a negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group; and/or
an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement. In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group; and/or
a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement. In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group.

Once a plurality of markers have been identified for use in a marker panel, such a panel may be used to evaluate an individual, e.g., for diagnostic, prognostic, and/or therapeutic purposes. In certain embodiments, concentrations of the individual markers can each be compared to a level (a “threshold”) that is preselected to rule in or out one or more particular diagnoses, prognoses, and/or therapy regimens. In these embodiments, correlating of each of the subject's selected marker level can comprise comparison to thresholds for each marker of interest that are indicative of a particular diagnosis. Similarly, by correlating the subject's marker levels to prognostic thresholds for each marker, the probability that the subject will suffer one or more future adverse outcomes may be determined.

In other embodiments, particular thresholds for one or more markers in a panel are not relied upon to determine if a profile of marker levels obtained from a subject are correlated to a particular diagnosis or prognosis. Rather, the present invention may utilize an evaluation of the entire profile of markers to provide a single result value (e.g., a “panel response” value expressed either as a numeric score or as a percentage risk). In such embodiments, an increase, decrease, or other change (e.g., slope over time) in a certain subset of markers may be sufficient to indicate a particular condition or future outcome in one patient, while an increase, decrease, or other change in a different subset of markers may be sufficient to indicate the same or a different condition or outcome in another patient.

In various embodiments, multiple determinations of one or more markers can be made, and a temporal change in the markers can be used to rule in or out one or more particular diagnoses and/or prognoses. For example, one or more markers may be determined at an initial time, and again at a second time, and the change (or lack thereof) in the marker level(s) over time determined. In such embodiments, an increase in the marker from the initial time to the second time may be indicative of a particular prognosis, of a particular diagnosis, etc. Likewise, a decrease in the marker from the initial time to the second time may be indicative of a particular prognosis, of a particular diagnosis, etc. In such a panel, the markers need not change in concert with one another. Temporal changes in one or more markers may also be used together with single time point marker levels to increase the discriminating power of marker panels. In yet another alternative, a “panel response” may be treated as a marker, and temporal changes in the panel response may be indicative of a particular prognosis, diagnosis, etc.

As discussed in detail herein, a plurality of markers may be combined, preferably to increase the predictive value of the analysis in comparison to that obtained from the markers individually. Such panels may comprise 2, 3, 4, 5, 6, 7, 8, 9, 10, 15, 20, or more or individual markers. The skilled artisan will also understand that diagnostic markers, differential diagnostic markers, prognostic markers, time of onset markers, etc., may be combined in a single assay or device. For example, certain markers measured by a device or instrument may be used provide a prognosis, while a different set of markers measured by the device or instrument may rule in and/or out particular therapies; each of these sets of markers may comprise unique markers, or may include markers that overlap with one or both of the other sets. Markers may also be commonly used for multiple purposes by, for example, applying a different set of analysis parameters (e.g., different midpoint, linear range window and/or weighting factor) to the marker(s) for the different purpose(s).

While exemplary panels are described herein, one or more markers may be replaced, added, or subtracted from these exemplary panels while still providing clinically useful results. Panels may comprise both specific markers of a condition of interest and/or non-specific markers (e.g., markers that are increased or decreased due to a condition of interest, but are also increased in other conditions). While certain markers may not individually be definitive in the methods described herein, a particular “fingerprint” pattern of changes may, in effect, act as a specific indicator of disease state. As discussed above, that pattern of changes may be obtained from a single sample, or may optionally consider temporal changes in one or more members of the panel (or temporal changes in a panel response value).

Use in Conjunction with a Treatment Regimen

Just as the potential causes of any particular nonspecific symptom may be a large and diverse set of conditions, the appropriate treatments for these potential causes may be equally large and diverse. However, once a diagnosis is obtained, the clinician can readily select a treatment regimen that is compatible with the diagnosis. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999.

In addition, since the methods and compositions described herein can provide prognostic information, the panels and markers of the present invention may be used to monitor a course of treatment. For example, improved or worsened prognostic state may indicate that a particular treatment is or is not efficacious. The term “theranostics” is used to describe the process of tailoring diagnostic therapy for an individual based on test results obtained for the particular individual. Theranostics go beyond traditional diagnosis, which is only concerned with identifying the presence of a disease. Theranostics can include one or more of predicting risks of disease, diagnosing disease, stratifying patients for risk, and monitoring therapeutic response. The diagnostic and/or prognostic methods of the present invention may be advantageously integrated into a therapy regimen so that the characteristics of treatment received by the individual is, at least in part, guided by the results of the methods, thereby individualizing and optimizing the therapeutic regimen of the individual.

### Treatment and Prevention of Colorectal Cancer

Colorectal cancer is treated or prevented by administration to a subject suspected of having or known to have colorectal cancer or to be at risk of developing colorectal cancer of a compound that modulates (i.e., increases or decreases) the level or activity (i.e., function) of one or more CRCMPs that are differentially present in the serum of subjects having colorectal cancer compared with serum of subjects free from colorectal cancer. In one embodiment, colorectal cancer is treated or prevented by administering to a subject suspected of having or known to have colorectal cancer or to be at risk of developing colorectal cancer a compound that upregulates (i.e., increases) the level or activity (i.e., function) of one or more CRCMPs that are decreased in the serum of subjects having colorectal cancer. In another embodiment, a compound is administered that downregulates the level or activity (i.e., function) of one or more CRCMPs that are increased in the serum of subjects having colorectal cancer. Examples of such a compound include but are not limited to: a CRCMP, CRCMP fragments and CRCMP-related polypeptides; nucleic acids encoding a CRCMP, a CRCMP fragment and a CRCMP-related polypeptide (e.g. for use in gene therapy); and, for those CRCMP or CRCMP-related polypeptides with enzymatic activity, compounds or molecules known to modulate that enzymatic activity. Other compounds that can be used, e.g. CRCMP agonists, can be identified using in in vitro assays.

Colorectal cancer is also treated or prevented by administration to a subject suspected of having or known to have colorectal cancer or to be at risk of developing colorectal cancer of a compound that downregulates the level or activity of one or more CRCMPs that are increased in the serum of subjects having colorectal cancer. In another embodiment, a compound is administered that upregulates the level or activity of one or more CRCMPs that are decreased in the serum of subjects having colorectal cancer. Examples of such a compound include, but are not limited to, CRCMP antisense oligonucleotides, ribozymes, antibodies (or other affinity reagents such as Affibodies, Nanobodies or Unibodies) directed against a CRCMP, and compounds that inhibit the enzymatic activity of a CRCMP. Other useful compounds e.g. CRCMP antagonists and small molecule CRCMP antagonists, can be identified using in vitro assays.

In a preferred embodiment, therapy or prophylaxis is tailored to the needs of an individual subject. Thus, in specific embodiments, compounds that promote the level or function of one or more CRCMPs are therapeutically or prophylactically administered to a subject suspected of having or known to have colorectal cancer, in whom the levels or functions of said one or more CRCMPs are absent or are decreased relative to a control or normal reference range. In further embodiments, compounds that promote the level or function of one or more CRCMPs are therapeutically or prophylactically administered to a subject suspected of having or known to have colorectal cancer in whom the levels or functions of said one or more CRCMPs are increased relative to a control or to a reference range. In further embodiments, compounds that decrease the level or function of one or more CRCMPs are therapeutically or prophylactically administered to a subject suspected of having or known to have colorectal cancer in whom the levels or functions of said one or more CRCMPs are increased relative to a control or to a reference range. In further embodiments, compounds that decrease the level or function of one or more CRCMPs are therapeutically or prophylactically administered to a subject suspected of having or known to have colorectal cancer in whom the levels or functions of said one or more CRCMPs are decreased relative to a control or to a reference range. The change in CRCMP function or level due to the administration of such compounds can be readily detected, e.g., by obtaining a sample (e.g., blood or urine) and assaying in vitro the levels or activities of said CRCMPs, or the levels of mRNAs encoding said CRCMPs, or any combination of the foregoing. Such assays can be performed before and after the administration of the compound as described herein.

The compounds of the invention include but are not limited to any compound, e.g., a small organic molecule, protein, peptide, antibody (or other affinity reagent such as an Affibody, Nanobody or Unibody), nucleic acid, etc. that restores the CRCMP profile towards normal. The compounds of the invention may be given in combination with any other compound.

### Immunotherapy and Prevention of Colorectal Cancer

CRCMPs may be useful in immunogenic compositions (suitably vaccines) for raising immune responses against proteins that may cause, sustain colorectal cancer or lead to metastases. Thus there is provided according to the invention a vaccine comprising one or more soluble polypeptides derived from a protein selected from the list consisting of proteins defined by SEQ ID Nos 1-18 and/or one or more antigenic or immunogenic fragments thereof. There is also provided an immunogenic composition which comprises one or more soluble polypeptides derived from a protein selected from the list consisting of proteins defined by SEQ ID Nos 1-18 and/or one or more antigenic or immunogenic fragments thereof, and one or more suitable adjuvants. Such a composition is useful in inducing an immune response in a subject, e.g. a human. There is also provided the use of one or more soluble polypeptides derived from a protein selected from the list consisting of proteins defined by SEQ ID Nos 1-18 and/or one or more antigenic or immunogenic fragments thereof in the preparation of an immunogenic composition, preferably a vaccine. There is also provided a method for the treatment or prophylaxis of colorectal cancer in a subject, or of vaccinating a subject against colorectal cancer, which comprises the step of administering to the subject an effective amount of one or more soluble polypeptides derived from a protein selected from the list consisting of proteins defined by SEQ ID Nos 1-18 and/or one or more antigenic or immunogenic fragments thereof, preferably as a vaccine.

Suitable immunogenic fragments are at least 10 amino acids in length e.g. at least 12 amino acids in length suitably at least 15 amino acids in length.

Suitable adjuvants will be well known to a person skilled in the art (see Vaccine design—the subunit and adjuvant approach (1995) Plenum Press).

### Determining Abundance of the CRCMPs by Imaging Technology

An advantage of determining abundance of the CRCMPs by imaging technology may be that such a method is non-invasive (save that reagents may need to be administered) and there is no need to extract a sample from the subject.

Suitable imaging technologies include positron emission tomography (PET) and single photon emission computed tomography (SPECT). Visualisation of the CRCMPs using such techniques requires incorporation or binding of a suitable label e.g. a radiotracer such as ¹⁸F, ¹¹C or ¹²³I (see e.g. NeuroRx—The Journal of the American Society for Experimental NeuroTherapeutics (2005) 2(2), 348-360 and idem pages 361-371 for further details of the techniques). Radiotracers or other labels may be incorporated into a CRCMP by administration to the subject (e.g. by injection) of a suitably labelled specific ligand. Alternatively they may be incorporated into a binding affinity reagent (antibody, Affibody, Nanobody, Unibody etc.) specific for the CRCMP which may be administered to the subject (e.g. by injection). For discussion of use of Affibodies for imaging see e.g. Orlova A, Magnusson M, Eriksson T L, Nilsson M, Larsson B, Hoiden-Guthenberg I, Widstrom C, Carlsson J, Tolmachev V, Stahl S, Nilsson F Y, Tumor imaging using a picomolar affinity HER2 binding affibody molecule, Cancer Res. 2006 Apr. 15; 66(8):4339-48).

### Diagnosis and Treatment of Colorectal Cancer Using Immunohistochemistry

Immunohistochemistry is an excellent detection technique and may therefore be very useful in the diagnosis and treatment of colorectal cancer. Immunohistochemistry may be used to detect, diagnose, or monitor colorectal cancer through the localization of CRCMP antigens in tissue sections by the use of labeled antibodies (or other affinity reagents such as Affibodies, Nanobodies or Unibodies), derivatives and analogs thereof, which specifically bind to a CRCMP, as specific reagents through antigen-antibody interactions that are visualized by a marker such as fluorescent dye, enzyme, radioactive element or colloidal gold.

The advancement of monoclonal antibody technology has been of great significance in assuring the place of immunohistochemistry in the modern accurate microscopic diagnosis of human neoplasms. The identification of disseminated neoplastically transformed cells by immunohistochemistry allows for a clearer picture of cancer invasion and metastasis, as well as the evolution of the tumour cell associated immunophenotype towards increased malignancy. Future antineoplastic therapeutical approaches may include a variety of individualized immunotherapies, specific for the particular immunophenotypical pattern associated with each individual patient's neoplastic disease. For further discussion see e.g. Bodey B, The significance of immunohistochemistry in the diagnosis and therapy of neoplasms, Expert Opin Biol Ther. 2002 April; 2(4):371-93.

Preferred features of each aspect of the invention are as for each of the other aspects mutatis mutandis. The prior art documents mentioned herein are incorporated to the fullest extent permitted by law.

### Example 1

### Identification of Membrane Proteins Expressed in Colorectal Cancer Tissue Samples

Using the following Reference Protocol, membrane proteins extracted from colorectal tissue samples were separated by 1D gel and analysed.

### 1.1 Materials and Methods

### 1.1.1—Plasma Membrane Fractionation

The cells recovered from the epithelium of a colorectal adenocarcinoma were lysed and submitted to centrifugation at 1000 G. The supernatant was taken, and it was subsequently centrifuged at 3000 G. Once again, the supernatant was taken, and it was then centrifuged at 100 000 G.

The resulting pellet was recovered and put on 15-60% sucrose gradient.

A Western blot was used to identify sub cellular markers, and the Plasma Membrane fractions were pooled.

The pooled solution was either run directly on 1D gels (see section 1.1.4 below), or further fractionated into heparin binding and nucleotide binding fractions as described below.

### 1.1.2—Plasma Membrane Heparin-Binding Fraction

The pooled solution from 1a above was applied to an Heparin column, eluted from column and run on 1D gels (see section id below).

### 1.1.3—Plasma Nucleotide-Binding Fraction

The pooled solution from 1.1.1 above was applied to a Cibacrom Blue 3GA column, eluted from column and run on 1D gels (see section 1.1.4 below).

### 1.1.4—1D Gel Technology

Protein or membrane pellets were solubilised in 1D sample buffer (1-2 μg/μl). The sample buffer and protein mixture was then heated to 95° C. for 3 min.

A 9-16% acrylamide gradient gel was cast with a stacking gel and a stacking comb according to the procedure described in Ausubel F. M. et al., eds., 1989, Current Protocols in Molecular Biology, Vol. II, Green Publishing Associates, Inc., and John Wiley & Sons, Inc., New York, section 10.2, incorporated herein by reference in its entirety.

30-50 micrograms of the protein mixtures obtained from detergent and the molecular weight standards (66, 45, 31, 21, 14 kDa) were added to the stacking gel wells using a 10 microlitre pipette tip and the samples run at 40 mA for 5 hours.

The plates were then prised open, the gel placed in a tray of fixer (10% acetic acid, 40% ethanol, 50% water) and shaken overnight. Following this, the gel was primed by 30 minutes shaking in a primer solution (7.5% acetic acid (75 mls), 0.05% SDS (5 mls of 10%)). The gel was then incubated with a fluorescent dye (7.5% acetic acid, 0.06% OGS in-house dye (600 μl) with shaking for 3 hrs. Sypro Red (Molecular Probes, Inc., Eugene, Oreg.) is a suitable dye for this purpose. A preferred fluorescent dye is disclosed in U.S. application Ser. No. 09/412,168, filed on Oct. 5, 1999, which is incorporated herein by reference in its entirety.

A computer-readable output was produced by imaging the fluorescently stained gels with an Apollo 3 scanner (Oxford Glycosciences, Oxford, UK). This scanner is developed from the scanner described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. The latest embodiment of this instrument includes the following improvements: The gel is transported through the scanner on a precision lead-screw drive system. This is preferable to laying the glass plate on the belt-driven system that is defined in the Basiji thesis as it provides a reproducible means of accurately transporting the gel past the imaging optics.

The gel is secured into the scanner against three alignment stops that rigidly hold the glass plate in a known position. By doing this in conjunction with the above precision transport system and the fact that the gel is bound to the glass plate, the absolute position of the gel can be predicted and recorded. This ensures that accurate co-ordinates of each feature on the gel can be communicated to the cutting robot for excision. This cutting robot has an identical mounting arrangement for the glass plate to preserve the positional accuracy.

The carrier that holds the gel in place has integral fluorescent markers (Designated M1, M2, M3) that are used to correct the image geometry and are a quality control feature to confirm that the scanning has been performed correctly.

The optical components of the system have been inverted. The laser, mirror, waveguide and other optical components are now above the glass plate being scanned. The embodiment of the Basiji thesis has these underneath. The glass plate is therefore mounted onto the scanner gel side down, so that the optical path remains through the glass plate. By doing this, any particles of gel that may break away from the glass plate will fall onto the base of the instrument rather than into the optics.

In scanning the gels, they were removed from the stain, rinsed with water and allowed to air dry briefly and imaged on the Apollo 3. After imaging, the gels were sealed in polyethylene bags containing a small volume of staining solution, and then stored at 4° C.

Apparent molecular weights were calculated by interpolation from a set of known molecular weight markers run alongside the samples.

### 1.1.5—Recovery and Analysis of Selected Proteins

Proteins were robotically excised from the gels by the process described in U.S. Pat. No. 6,064,754, Sections 5.4 and 5.6, 5.7, 5.8 (incorporated herein by reference), as is applicable to 1D-electrophoresis, with modification to the robotic cutter as follows: the cutter begins at the top of the lane, and cuts a gel disc 1.7 mm in diameter from the left edge of the lane. The cutter then moves 2 mm to the right, and 0.7 mm down and cuts a further disc. This is then repeated. The cutter then moves back to a position directly underneath the first gel cut, but offset by 2.2 mm downwards, and the pattern of three diagonal cuts are repeated. This is continued for the whole length of the gel.

NOTE: If the lane is observed to broaden significantly then a correction can be made also sideways i.e instead of returning to a position directly underneath a previous gel cut, the cut can be offset slightly to the left (on the left of the lane) and/or the right (on the right of the lane). The proteins contained within the gel fragments were processed to generate tryptic peptides; partial amino acid sequences of these peptides were determined by mass spectroscopy as described in WO98/53323 and application Ser. No. 09/094,996, filed Jun. 15, 1998.

Proteins were processed to generate tryptic digest peptides. Tryptic peptides were analyzed by mass spectrometry using a PerSeptive Biosystems Voyager-DETM STR Matrix-Assisted Laser Desorption Ionization Time-of-Flight (MALDI-TOF) mass spectrometer, and selected tryptic peptides were analyzed by tandem mass spectrometry (MS/MS) using a Micromass Quadrupole Time-of-Flight (Q-TOF) mass spectrometer (Micromass, Altrincham, U.K.) equipped with a Nanoflow™ electrospray Z-spray source. For partial amino acid sequencing and identification of CRCMPs uninterpreted tandem mass spectra of tryptic peptides were searched using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database, and a mass increment for all Cys residues to account for carbamidomethylation. The database searched was a database constructed of protein entries in the non-redundant database held by the National Centre for Biotechnology Information (NCBI) which is accessible at http://www.ncbi.nlm.nih.gov/. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662).

### 1.1.6—Discrimination of Colorectal Cancer Associated Proteins

The process to identify the CRCMPs uses the peptide sequences obtained experimentally by mass spectrometry described above of naturally occurring human proteins to identify and organize coding exons in the published human genome sequence.

Recent dramatic advances in defining the chemical sequence of the human genome have led to the near completion of this immense task (Venter, J. C. et al. (2001). The sequence of the human genome. Science 16: 1304-51; International Human Genome Sequencing Consortium. (2001). Initial sequencing and analysis of the human genome Nature 409: 860-921). There is little doubt that this sequence information will have a substantial impact on our understanding of many biological processes, including molecular evolution, comparative genomics, pathogenic mechanisms and molecular medicine. For the full medical value inherent in the sequence of the human genome to be realised, the genome needs to be ‘organised’ and annotated. By this, is meant at least the following three things. (i) The assembly of the sequences of the individual portions of the genome into a coherent, continuous sequence for each chromosome. (ii) The unambiguous identification of those regions of each chromosome that contain genes. (iii) Determination of the fine structure of the genes and the properties of its mRNA and protein products. While the definition of a ‘gene’ is an increasingly complex issue (H Pearson: What is a gene? Nature (2006) 24: 399-401)), what is of immediate interest for drug discovery and development is a catalogue of those genes that encode functional, expressed proteins. A subset of these genes will be involved in the molecular basis of most if not all pathologies. Therefore an important and immediate goal for the pharmaceutical industry is to identify all such genes in the human genome and describe their fine structure.

### Processing and Integration of Peptide Masses, Peptide Signatures, ESTs and Public Domain Genomic Sequence Data to Form OGAP® Database

Discrete genetic units (exons, transcripts and genes) were identified using the following sequential steps:
1. A ‘virtual transcriptome’ is generated, containing the tryptic peptides which map to the human genome by combining the gene identifications available from Ensembl and various gene prediction programs. This also incorporates SNP data (from dbSNP) and all alternate splicing of gene identifications. Known contaminants were also added to the virtual transcriptome.
2. All tandem spectra in the OGeS Mass Spectrometry Database are interpreted in order to produce a peptide that can be mapped to one in the virtual transcriptome. A set of automated spectral interpretation algorithms were used to produce the peptide identifications.
3. The set of all mass-matched peptides in the OGeS Mass Spectrometry Database is generated by searching all peptides from transcripts hit by the tandem peptides using a tolerance based on the mass accuracy of the mass spectrometer, typically 20 ppm.
4. All tandem and mass-matched peptides are combined in the form of “protein clusters”. This is done using a recursive process which groups sequences into clusters based on common peptide hits. Biological sequences are considered to belong to the same cluster if they share one or more tandem or mass-matched peptide.
5. After initial filtering to screen out incorrectly identified peptides, the resulting clusters are then mapped on the human genome.
6. The protein clusters are then aggregated into regions that define preliminary gene boundaries using their proximity and the co-observation of peptides within protein clusters. Proximity is defined as the peptide being within 80,000 nucleotides on the same strand of the same chromosome. Various elimination rules, based on cluster observation scoring and multiple mapping to the genome are used to refine the output. The resulting ‘confirmed genes’ are those which best account for the peptides and masses observed by mass spectrometry in each cluster. Nominal co-ordinates for the gene are also an output of this stage.
7. The best set of transcripts for each confirmed gene are created from the protein clusters, peptides, ESTs, candidate exons and molecular weight of the original protein spot.
8. Each identified transcript was linked to the sample providing the observed peptides
9. Use of an application for viewing and mining the data. The result of steps 1-8 was a database containing genes, each of which consisted of a number of exons and one or more transcripts. An application was written to display and search this integrated genome/proteome data. Any features (OMIM disease locus, InterPro etc.) that had been mapped to the same Golden Path co-ordinate system by Ensembl could be cross-referenced to these genes by coincidence of location and fine structure.

### Results

The process was used to generate approximately 1 million peptide sequences to identify protein-coding genes and their exons resulted in the identification of protein sequences for 18083 genes across 67 different tissues and 57 diseases including 506 genes in Bladder cancer, 4,713 genes in Breast cancer, 767 genes in Burkitt's lymphoma, 1,372 genes in Cervical cancer, 949 genes in colorectal cancer, 1,783 genes in Hepatocellular cancer, 2,425 genes in CLL, 978 genes in Lung cancer, 1,764 genes in Melanoma, 1,033 genes in Ovarian Cancer, 2,961 genes in Pancreatic cancer and 3,308 genes in Prostate cancer illustrated here by the list of proteins isolated and identified from colorectal cancer samples. Following comparison of the experimentally determined sequences with sequences in the OGAP® database, the CRCMPs listed in the tables showed a high degree of specificity to colorectal cancer indicative of the prognostic and diagnostic nature.

### 1.2 Results

These experiments identified Colorectal Cancer-associated features corresponding to 18 different genes, as listed in Table 1. The source of each feature according to the fractionation protocols described above is detailed in Table 4 below.

TABLE 4
Origins of the Features detected by 1D gel

| | | Plasma | Plasma |
|---|---|---|---|
| | Plasma | Membrane | Membrane |
| | Membrane | Heparin binding | Nucleotide |
| CRCMP # | Fractionation 1D | fraction | binding fraction |
| 1 | ✓ | | ✓ |
| 2 | ✓ | | ✓ |
| 5 | | | ✓ |
| 6 | ✓ | | |
| 7 | ✓ | | |
| 8 | | ✓ | |
| 9 | ✓ | | |
| 10 | ✓ | | |
| 12 | | ✓ | ✓ |
| 14 | | ✓ | |
| 17 | ✓ | | ✓ |
| 18 | ✓ | ✓ | ✓ |
| 19 | ✓ | | |
| 20 | ✓ | | |
| 22 | ✓ | ✓ | ✓ |
| 23 | | | ✓ |
| 25 | ✓ | ✓ | ✓ |
| 26 | ✓ | | |

### Example 2

### Identification of the Soluble Forms of the Membrane Proteins Expressed in Colorectal Cancer Tissue Samples

Using the following exemplary and non-limiting procedure, serum was analysed by isoelectric focusing followed by SDS-PAGE and the proteins corresponding to the features identified in Example 1 above were characterised in their circulating forms.

### 2.1 Materials and Methods

### 2.1.1 Sample Preparation

A protein assay (Pierce BCA Cat # 23225) was performed on each serum sample as received. Prior to protein separation, each sample was processed for selective depletion of certain proteins, in order to enhance and simplify protein separation and facilitate analysis by removing proteins that may interfere with or limit analysis of proteins of interest. See International Patent Application No. PCT/GB99/01742, filed Jun. 1, 1999, which is incorporated by reference in its entirety, with particular reference to pages 3 and 6.

Removal of albumin, haptoglobin, transferrin and immunoglobin G (IgG) from serum (“serum depletion”) was achieved by an affinity chromatography purification step in which the sample was passed through a series of ‘Hi-Trap’ columns containing immobilized antibodies for selective removal of albumin, haptoglobin and transferrin, and protein G for selective removal of immunoglobin G. Two affinity columns in a tandem assembly were prepared by coupling antibodies to protein G-sepharose contained in Hi-Trap columns (Protein G-Sepharose Hi-Trap columns (1 ml) Pharmacia Cat. No. 17-0404-01). This was done by circulating the following solutions sequentially through the columns: (1) Dulbecco's Phosphate Buffered Saline (Gibco BRL Cat. No. 14190-094); (2) concentrated antibody solution; (3) 200 mM sodium carbonate buffer, pH 8.35; (4) cross-linking solution (200 mM sodium carbonate buffer, pH 8.35, 20 mM dimethylpimelimidate); and (5) 500 mM ethanolamine, 500 mM NaCl. A third (un-derivatised) protein G Hi-Trap column was then attached to the lower end of the tandem column assembly.

The chromatographic procedure was automated using an Akta Fast Protein Liquid Chromatography (FPLC) System such that a series of up to seven runs could be performed sequentially. The samples were passed through the series of 3 Hi-Trap columns in which the affinity chromatography media selectively bind the above proteins thereby removing them from the sample. Fractions (typically 3 ml per tube) were collected of unbound material (“Flowthrough fractions”) that eluted through the column during column loading and washing stages and of bound proteins (“Bound/Eluted fractions”) that were eluted by step elution with Immunopure Gentle Ag/Ab Elution Buffer (Pierce Cat. No. 21013). The eluate containing unbound material was collected in fractions which were pooled, desalted/concentrated by centrifugal ultrafiltration and stored to await further analysis by 2D PAGE.

A volume of depleted serum containing approximately 300 μg of total protein was aliquoted and an equal volume of 10% (w/v) SDS (Fluka 71729), 2.3% (w/v) dithiothreitol (BDH 443852A) was added. The sample was heated at 95° C. for 5 mins, and then allowed to cool to 20° C. 125 μl of the following buffer was then added to the sample:

8M urea (BDH 452043w)

4% CHAPS (Sigma C3023)

65 mM dithiotheitol (DTT)

2% (v/v) Resolytes 3.5-10 (BDH 44338 2x)

This mixture was vortexed, and centrifuged at 13000 rpm for 5 mins at 15° C., and the supernatant was separated by isoelectric focusing as described below.

### 2.1.2 Isoelectric Focusing

Isoelectric focusing (IEF), was performed using the Immobiline® DryStrip Kit (Pharmacia BioTech), following the procedure described in the manufacturer's instructions, see Instructions for Immobiline® DryStrip Kit, Pharmacia, # 18-1038-63, Edition AB (incorporated herein by reference in its entirety). Immobilized pH Gradient (IPG) strips (18 cm, pH 3-10 non-linear strips; Pharmacia Cat. # 17-1235-01) were rehydrated overnight at 20° C. in a solution of 8M urea, 2% (w/v) CHAPS, 10 mM DTT, 2% (v/v) Resolytes 3.5-10, as described in the Immobiline DryStrip Users Manual. For IEF, 50 μl of supernatant (prepared as above) was loaded onto a strip, with the cup-loading units being placed at the basic end of the strip. The loaded gels were then covered with mineral oil (Pharmacia 17-3335-01) and a voltage was immediately applied to the strips according to the following profile, using a Pharmacia EPS3500XL power supply (Cat 19-3500-01):

Initial voltage=300V for 2 hrs

Linear Ramp from 300V to 3500V over 3 hrs

Hold at 3500V for 19 hrs

For all stages of the process, the current limit was set to 10 mA for 12 gels, and the wattage limit to 5 W. The temperature was held at 20° C. throughout the run.

### 2.1.3 Gel Equilibration and SDS-PAGE

After the final 19 hr step, the strips were immediately removed and immersed for 10 mins at 20° C. in a first solution of the following composition: 6M urea; 2% (w/v) DTT; 2% (w/v) SDS; 30% (v/v) glycerol (Fluka 49767); 0.05M Tris/HCl, pH 6.8 (Sigma Cat T-1503). The strips were removed from the first solution and immersed for 10 mins at 20° C. in a second solution of the following composition: 6M urea; 2% (w/v) iodoacetamide (Sigma 1-6125); 2% (w/v) SDS; 30% (v/v) glycerol; 0.05M Tris/HCl, pH 6.8. After removal from the second solution, the strips were loaded onto supported gels for SDS-PAGE according to Hochstrasser et al., 1988, Analytical Biochemistry 173: 412-423 (incorporated herein by reference in its entirety), with modifications as specified below.

### 2.1.4 Preparation of Supported Gels

The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of γ-methacryl-oxypropyltrimethoxysilane in ethanol (BindSilane™; Pharmacia Cat. # 17-1330-01). The front plate was treated with (RepelSilane™ Pharmacia Cat. # 17-1332-01) to reduce adhesion of the gel. Excess reagent was removed by washing with water, and the plates were allowed to dry. At this stage, both as identification for the gel, and as a marker to identify the coated face of the plate, an adhesive bar-code was attached to the back plate in a position such that it would not come into contact with the gel matrix.

The dried plates were assembled into a casting box with a capacity of 13 gel sandwiches. The front and back plates of each sandwich were spaced by means of 1 mm thick spacers, 2.5 cm wide. The sandwiches were interleaved with acetate sheets to facilitate separation of the sandwiches after gel polymerization. Casting was then carried out according to Hochstrasser et al., op. cit.

A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (Cat. # 10677). The cross-linking agent was PDA (BioRad 161-0202), at a concentration of 2.6% (w/w) of the total starting monomer content. The gel buffer was 0.375M Tris/HCl, pH 8.8. The polymerization catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerize at 20° C. overnight, and then stored individually at 4° C. in sealed polyethylene bags with 6 ml of gel buffer, and were used within 4 weeks.

### 2.1.5 SDS-PAGE

A solution of 0.5% (w/v) agarose (Fluka Cat 05075) was prepared in running buffer (0.025M Tris, 0.198M glycine (Fluka 50050), 1% (w/v) SDS, supplemented by a trace of bromophenol blue). The agarose suspension was heated to 70° C. with stirring, until the agarose had dissolved. The top of the supported 2nd D gel was filled with the agarose solution, and the equilibrated strip was placed into the agarose, and tapped gently with a palette knife until the gel was intimately in contact with the 2nd D gel. The gels were placed in the 2nd D running tank, as described by Amess et al., 1995, Electrophoresis 16: 1255-1267 (incorporated herein by reference in its entirety). The tank was filled with running buffer (as above) until the level of the buffer was just higher than the top of the region of the 2nd D gels which contained polyacrylamide, so as to achieve efficient cooling of the active gel area. Running buffer was added to the top buffer compartments formed by the gels, and then voltage was applied immediately to the gels using a Consort E-833 power supply. For 1 hour, the gels were run at 20 mA/gel. The wattage limit was set to 150 W, for a tank containing 6 gels, and the voltage limit was set to 600V. After 1 hour, the gels were then run at 40 mA/gel, with the same voltage and wattage limits as before, until the bromophenol blue line was 0.5 cm from the bottom of the gel. The temperature of the buffer was held at 16° C. throughout the run.

### 2.1.6 Staining

Upon completion of the electrophoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016×), 50% (v/v) water (MilliQ-Millipore), which was continuously circulated over the gels. After an overnight incubation, the fixative was drained from the tank, and the gels were primed by immersion in 7.5% (v/v) acetic acid, 0.05% (w/v) SDS, 92.5% (v/v) water for 30 mins. The priming solution was then drained, and the gels were stained by complete immersion for 4 hours in a staining solution of Sypro Red (Molecular Probes, Inc., Eugene, Oreg.). Alternative dyes which can be used for this purpose are described in U.S. patent application Ser. No. 09/412,168, filed Oct. 5, 1999, and incorporated herein by reference in its entirety.

### 2.1.7 Imaging of the Gel

A computer-readable output was produced by imaging the fluorescently stained gels with the Apollo 2 scanner (Oxford Glycosciences, Oxford, UK). This scanner has a gel carrier with four integral fluorescent markers (Designated M1, M2, M3, M4) that are used to correct the image geometry and are a quality control feature to confirm that the scanning has been performed correctly.

For scanning, the gels were removed from the stain, rinsed with water and allowed to air dry briefly, and imaged on the Apollo 2. After imaging, the gels were sealed in polyethylene bags containing a small volume of staining solution, and then stored at 4° C.

### 2.1.8 Digital Analysis of the Data

The data were processed as described in U.S. Pat. No. 6,064,654, (published as WO 98/23950) at Sections 5.4 and 5.5 (incorporated herein by reference), as set forth more particularly below.

The output from the scanner was first processed using the MELANIE® II 2D PAGE analysis program (Release 2.2, 1997, BioRad Laboratories, Hercules, Calif., Cat. # 170-7566) to autodetect the registration points, M1, M2, M3 and M4; to autocrop the images (i.e., to eliminate signals originating from areas of the scanned image lying outside the boundaries of the gel, e.g. the reference frame); to filter out artifacts due to dust; to detect and quantify features; and to create image files in GIF format. Features were detected using the following parameters:

Smooths=2

Laplacian threshold 50

Partials threshold 1

Saturation=100

Peakedness=0

Minimum Perimeter=10

### 2.1.9 Assignment of pI and MW Values

Landmark identification was used to determine the pI and MW of features detected in the images. Sixteen landmark features were identified in a standard serum image.

As many of these landmarks as possible were identified in each gel image of the dataset. Each feature in the study gels was then assigned a pI value by linear interpolation or extrapolation (using the MELANIE®-II software) to the two nearest landmarks, and was assigned a MW value by linear interpolation or extrapolation (using the MELANIE®-II software) to the two nearest landmarks.

2.1.10 Matching with Primary Master Image

Images were edited to remove gross artifacts such as dust, to reject images which had gross abnormalities such as smearing of protein features, or were of too low a loading or overall image intensity to allow identification of more than the most intense features, or were of too poor a resolution to allow accurate detection of features. Images were then compared by pairing with one common image from the whole sample set. This common image, the “primary master image”, was selected on the basis of protein load (maximum load consistent with maximum feature detection), a well resolved myoglobin region, (myoglobin was used as an internal standard), and general image quality. Additionally, the primary master image was chosen to be an image which appeared to be generally representative of all those to be included in the analysis. (This process by which a primary master gel was judged to be representative of the study gels was rechecked by the method described below and in the event that the primary master gel was seen to be unrepresentative, it was rejected and the process repeated until a representative primary master gel was found.)

Each of the remaining study gel images was individually matched to the primary master image such that common protein features were paired between the primary master image and each individual study gel image as described below.

### 2.1.11 Cross-Matching Between Samples

The geometry of each study gel was adjusted for maximum alignment between its pattern of protein features, and that of the primary master, as follows. Each of the study gel images was individually transformed into the geometry of the primary master image using a multi-resolution warping procedure. This procedure corrects the image geometry for the distortions brought about by small changes in the physical parameters of the electrophoresis separation process from one sample to another. The observed changes are such that the distortions found are not simple geometric distortions, but rather a smooth flow, with variations at both local and global scale.

The fundamental principle in multi-resolution modeling is that smooth signals may be modeled as an evolution through ‘scale space’, in which details at successively finer scales are added to a low resolution approximation to obtain the high resolution signal. This type of model is applied to the flow field of vectors (defined at each pixel position on the reference image) and allows flows of arbitrary smoothness to be modeled with relatively few degrees of freedom. Each image is first reduced to a stack, or pyramid, of images derived from the initial image, but smoothed and reduced in resolution by a factor of 2 in each direction at every level (Gaussian pyramid) and a corresponding difference image is also computed at each level, representing the difference between the smoothed image and its progenitor (Laplacian pyramid). Thus the Laplacian images represent the details in the image at different scales.

To estimate the distortion between any 2 given images, a calculation was performed at level 7 in the pyramid (i.e. after 7 successive reductions in resolution). The Laplacian images were segmented into a grid of 16×16 pixels, with 50% overlap between adjacent grid positions in both directions, and the cross correlation between corresponding grid squares on the reference and the test images was computed. The distortion displacement was then given by the location of the maximum in the correlation matrix. After all displacements had been calculated at a particular level, they were interpolated to the next level in the pyramid, applied to the test image, and then further corrections to the displacements were calculated at the next scale.

The warping process brought about good alignment between the common features in the primary master image, and the images for the other samples. The MELANIE® II 2D PAGE analysis program was used to calculate and record approximately 500-700 matched feature pairs between the primary master and each of the other images. The accuracy of, this program was significantly enhanced by the alignment of the images in the manner described above. To improve accuracy still further, all pairings were finally examined by eye in the MelView interactive editing program and residual recognizably incorrect pairings were removed. Where the number of such recognizably incorrect pairings exceeded the overall reproducibility of the technology (as measured by repeat analysis of the same biological sample) the gel selected to be the primary master gel was judged to be insufficiently representative of the study gels to serve as a primary master gel. In that case, the gel chosen as the primary master gel was rejected, and different gel was selected as the primary master gel, and the process was repeated.

All the images were then added together to create a composite master image, and the positions and shapes of all the gel features of all the component images were super-imposed onto this composite master as described below.

Once all the initial pairs had been computed, corrected and saved, a second pass was performed whereby the original (unwarped) images were transformed a second time to the geometry of the primary master, this time using a flow field computed by smooth interpolation of the multiple tie-points defined by the centroids of the paired gel features. A composite master image was thus generated by initializing the primary master image with its feature descriptors. As each image was transformed into the primary master geometry, it was digitally summed pixel by pixel into the composite master image, and the features that had not been paired by the procedure outlined above were likewise added to the composite master image description, with their centroids adjusted to the master geometry using the flow field correction.

The final stage of processing was applied to the composite master image and its feature descriptors, which now represent all the features from all the images in the study transformed to a common geometry. The features were grouped together into linked sets or “clusters”, according to the degree of overlap between them. Each cluster was then given a unique identifying index, the molecular cluster index (MCI).

An MCI identifies a set of matched features on different images. Thus an MCI represents a protein or proteins eluting at equivalent positions in the 2D separation in different samples.

### 2.1.12. Construction of Profiles

After matching all component gels in the study to the final composite master image, the intensity of each feature was measured and stored. The end result of this analysis was the generation of a digital profile which contained, for each identified feature: 1) a unique identification code relative to corresponding feature within the composite master image (MCI), 2) the x, y coordinates of the features within the gel, 3) the isoelectric point (pI) of the Protein Isoforms, 4) the apparent molecular weight (MW) of the Protein Isoforms, 5) the signal value, 6) the standard deviation for each of the preceding measurements, and 7) a method of linking the MCI of each feature to the master gel to which this feature was matched. By virtue of a Laboratory Information Management System (LIMS), this MCI profile was traceable to the actual stored gel from which it was generated, so that proteins identified by computer analysis of gel profile databases could be retrieved. The LIMS also permitted the profile to be traced back to an original sample or patient.

### 2.1.13. Recovery and Analysis of Selected Proteins

Protein Isoforms were robotically excised and processed to generate tryptic digest peptides. Tryptic peptides were analyzed by mass spectrometry using a PerSeptive Biosystems Voyager-DETM STR Matrix-Assisted Laser Desorption Ionization Time-of-Flight (MALDI-TOF) mass spectrometer, and selected tryptic peptides were analyzed by tandem mass spectrometry (MS/MS) using a Micromass Quadrupole Time-of-Flight (Q-TOF) mass spectrometer (Micromass, Altrincham, U.K.), equipped with a Nanoflow™ electrospray Z-spray source. For partial amino acid sequencing and identification of Protein Isoforms uninterpreted tandem mass spectra of tryptic peptides were searched using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database, and a mass increment for all Cys residues to account for carbamidomethylation. The database searched was a database constructed of protein entries in the non-redundant database held by the National Centre for Biotechnology Information (NCBI) which is accessible at http://www.ncbi.nlm.nih.gov/. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662).

### 2.1.14—Discrimination of Colorectal Cancer Associated Proteins

The process described in Example 1 section 1.1.6 was employed to discriminate the colorectal cancer associated proteins in the experimental samples.

### 2.2 Results

These experiments identified the CRCMPs which are listed in Table 2.

### Example 3

### Evaluation of Colorectal Cancer Marker Proteins in Sandwich ELISA

Using the following Reference Protocol, the Colorectal Cancer Marker Proteins (CRCMPs) listed in Tables 1 and 2 were evaluated in a sandwich ELISA.

### 3.1 Materials and Methods

Antibodies for the sandwich ELISAs were developed at Biosite. Biotinylated antibody (primary antibody) was diluted into assay buffer (10 mM Tris, 150 mM NaCl, 1% BSA) to 2 ug/ml and added to 384 well neutravidin coated plate (Pierce Chemical Company, Rockford Ill.) and allowed to incubate at room temperature for 1 hour. Wells were then washed with wash buffer (20 mM Borate, 150 mM NaCl, 0.2% Tween 20). Samples and standards were added and allowed to incubate at room temperature for 1 hour. Wells again were washed. An antibody conjugated to fluorscein (secondary antibody) was diluted into assay buffer to 2 ug/ml and was then added to the plate and allowed to incubate at room temperature for 1 hour. Wells again were washed. Anti-fluorscein antibody conjugated to alkaline phosphatase, diluted 1/2338 into assay buffer, was added and allowed to incubate at room temperature for 1 hour. Final wash was then performed. Finally substrate (Promega Attophos Product#S1011, Promega Corporation, Madison, Wis.) was added and the plate was read immediately. All additions were 10 ul/well. The plate was washed 3 times between each addition and final wash was 9 times prior to the addition of substrate. Standards were prepared by spiking specific antigen into a normal serum patient pool. Reading was performed using a Tecan Spectrafluor plus (Tecan Inc, Mannedorf, Switzerland) in kinetic mode for 6 read cycles with excitation filter of 430 nm and an emission filter 570 nm emission. Slope of RFU/seconds was determined.

Final Box and ROC results were analyzed using Analyse-it General+Clinical Laboratory 1.73 (Analyse-it Software Ltd., Leeds England).

### 3.2 Results

These experiments identified CRCMPs of particular interest including, but not limited to, CRCMP#19 (SEQ ID No: 13), CRCMP#9 (SEQ ID No: 7), CRCMP#6 (SEQ ID No: 4), CRCMP#22 (SEQ ID No: 15) and CRCMP#10 (SEQ ID No: 8).

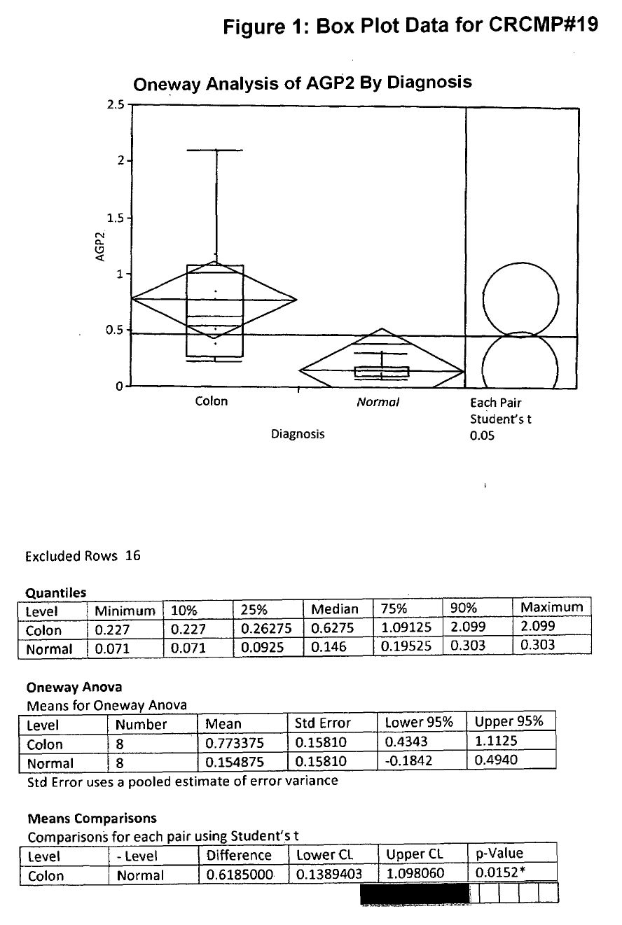

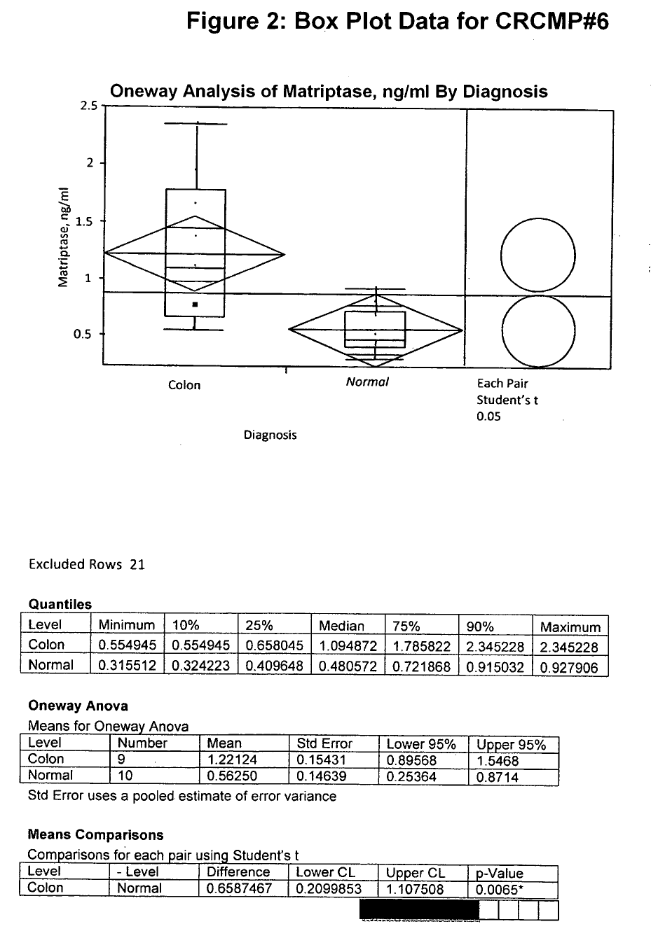

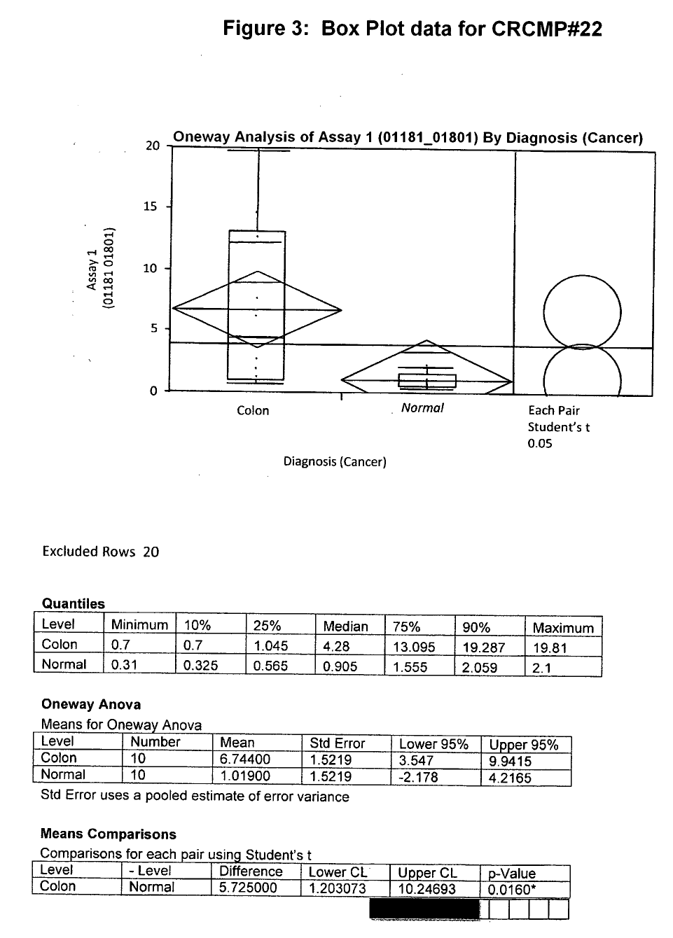

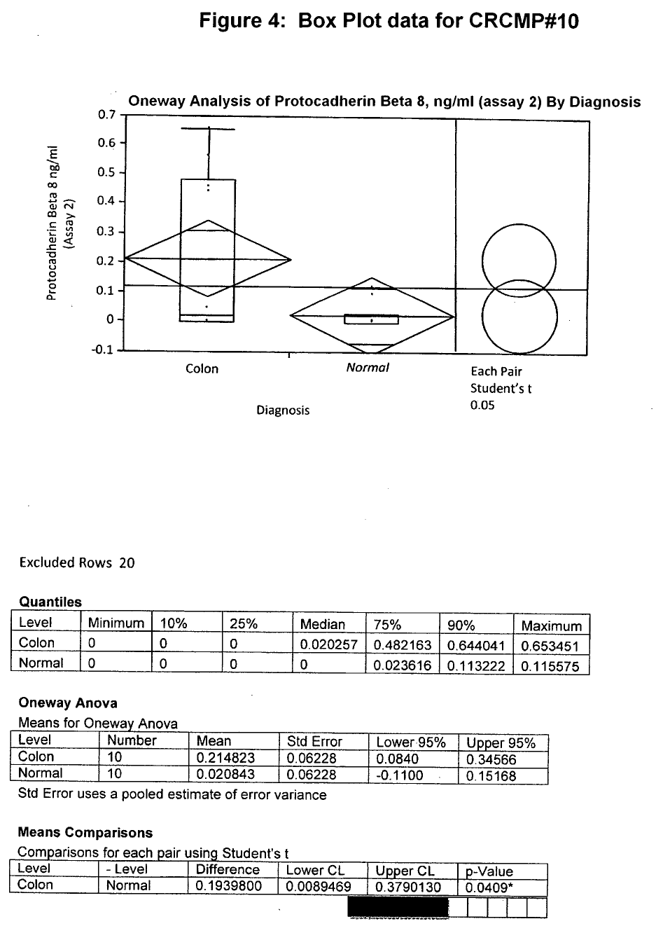

FIGS. 1-4 show Box plot data for CRCMP#19, CRCMP#6, CRCMP#22 and CRCMP#10 respectively. The vertical axes on these graphs are concentration of the CRCMP in ng/ml, except for FIG. 3 where the vertical axis is signal response. These data all show higher concentration of the CRCMP in colorectal cancer samples compared to normal samples, with significant p values, thereby indicating that CRCMP#19, CRCMP#6, CRCMP#22 and CRCMP#10 discriminate well between colorectal cancer and normal, making them good potential markers for colorectal cancer.

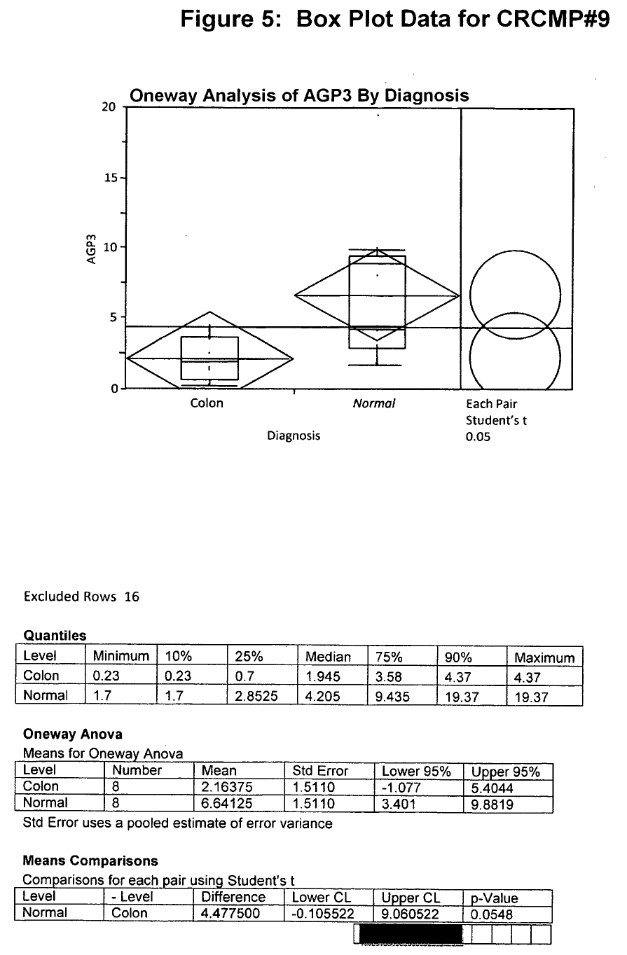

FIG. 5 shows Box plot data for CRCMP#9. The vertical axis on this graph is concentration of CRCMP#9 in ng/ml. These data show decreased concentration of CRCMP#9 in colorectal cancer samples compared to normal samples, with an almost significant p value, thereby indicating that CRCMP#9 discriminates well between colorectal cancer and normal, making it a good potential marker for colorectal cancer.

### Example 4

### Evaluation of Colorectal Cancer Marker Proteins in Multiplex Assay Using Luminex Technology

Using the following Reference Protocol, Colorectal Cancer Marker Proteins (CRCMPs) listed in Tables 1 and 2 were evaluated in a multiplex assay using the Luminex technology.

### 4.1 Materials and Methods

Each primary antibody was conjugated to a unique Luminex magnetic microsphere (Mug beads, Luminex Corporation, Austin, Tex.). Mag bead cocktail (50 ul) was added to a 96 black well round bottom Costar plate (Corning Incorporated, Corning N.Y.). Using a 96 well magnetic ring stand, the Mag beads were pulled down for 1 minute and washed with wash/assay buffer (PBS with 1% BSA and 0.02% Tween 20). 50 ul of sample or standard was added along with an additional 50 ul of wash/assay buffer and allowed to incubate on a shaker for 1 hour at room temperature. Plate was placed on magnetic ring stand and allowed to sit for 1 minute. Mag beads were then washed again. Biotin labeled antibody was then added at 50 ul per well with an additional 50 ul of wash/assay buffer and allowed to incubate on a shaker for 1 hour at room temperature. The plate again was placed on a magnetic stand and the Mag beads were washed. Streptavidin-RPE (Prozyme, San Leandro, Calif., Phycolin, Code#PJ31S) was diluted to 1 ug/ml in wash/assay buffer and 50 ul was added to each well along with an additional 50 ul of wash/assay buffer and allowed to incubate on a shaker for 1 hour at room temperature. Final wash was performed and the beads were re-suspended with 100 ul of wash/assay buffer and each well was then read in a Luminex 200 reader using Xponent software 3.0. All reagent dilutions were made in wash/assay buffer. Biotin-antibody varied for each assay to optimal concentration. Initial Mag bead amounts added were approximately 50,000 for each assay. Magnetic beads were allowed 1 minute pull down time prior to each wash. Each wash step was 3 times washed with 100 ul of wash/assay buffer. Assay standard curves were made in a normal donor patient serum pool. Luminex reader and Mag beads were used and prepared according to manufacturer guidelines. Standard curves were calculated using a 5 parameter log-logistic fit and each sample concentration was determined from this curve fit.

Final Box and ROC results were analyzed using Analyse-it General+Clinical Laboratory 1.73 (Analyse-it Software Ltd., Leeds England).

### 4.2 Results

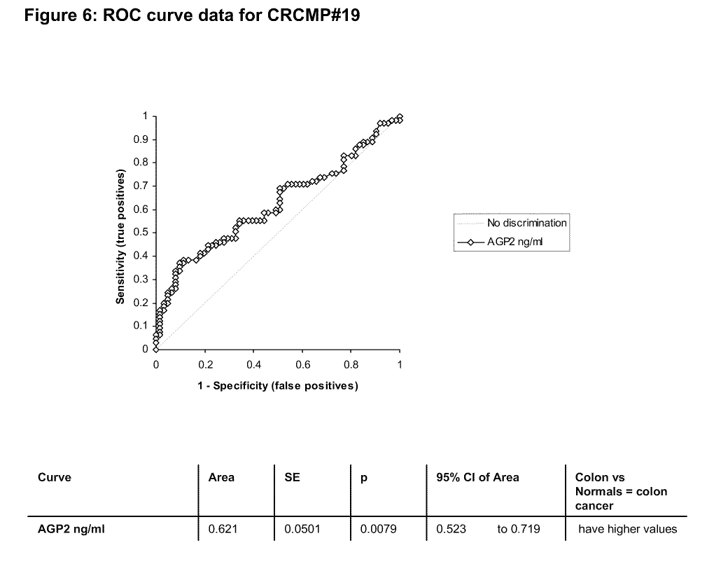

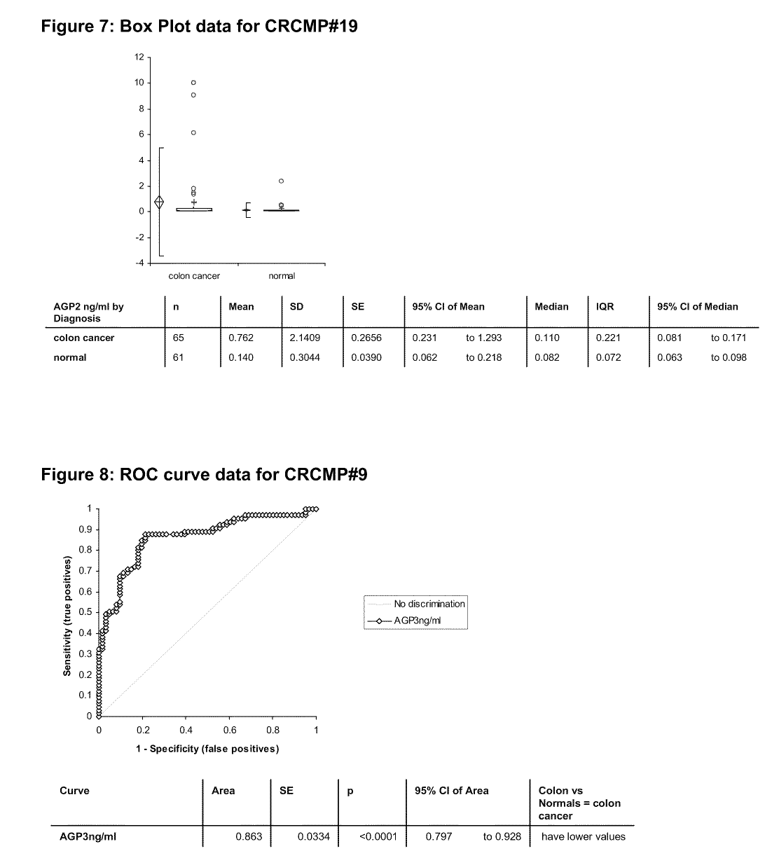

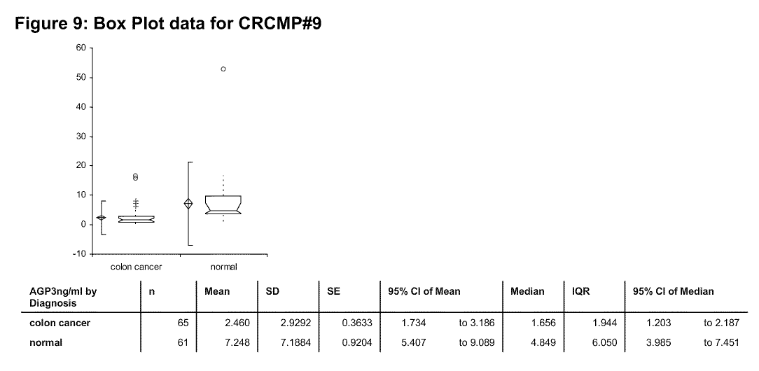

Experiments using 61 normal samples and 65 colorectal cancer samples resulted in further evidence for some of the CRCMPs of interest identified in Example 3 above, including, but not limited to, CRCMP#19 (SEQ ID No: 13) and CRCMP#9 (SEQ ID No: 7). FIG. 6 shows ROC curve data for CRCMP#19 and FIG. 7 shows Box plot data for CRCMP#19. FIG. 8 shows ROC curve data for CRCMP#9 and FIG. 9 shows Box plot data for CRCMP#9.

The ROC curves plot sensitivity (true positives) against 1-specificity (false positives). The area under the ROC curve is a measure of the probability that the measured marker level will allow correct identification of a disease or condition. An area of greater than 0.5 indicates that the marker can discriminate between disease and normal. This is the case in the data shown in FIG. 6 and FIG. 8 therefore indicating that both CRCMP#19 and CRCMP#9 are good potential markers to discriminate between colorectal cancer and normal. CRCMP#9 in particular has a high area under the curve and a very low p value indicating that it may be a particularly good marker for colorectal cancer.

The vertical axes on the box plots in FIG. 7 and FIG. 9 is concentration of the CRCMP in ng/ml. FIG. 7 shows higher concentration of CRCMP#19 in colorectal cancer samples than in normal samples whereas FIG. 9 shows lower concentration of CRCMP#9 in colorectal cancer samples than in normal samples. Both CRCMP#19 and CRCMP#9 show good discrimination between colorectal cancer and normal, indicating that these are both good potential markers for colorectal cancer.

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A method of diagnosing colorectal cancer in a subject, differentiating causes of colorectal cancer in a subject, guiding therapy in a subject suffering from colorectal cancer, assessing the risk of relapse in a subject suffering from colorectal cancer, or assigning a prognostic risk of one or more future clinical outcomes to a subject suffering from colorectal cancer, the method comprising:
(a) performing assays configured to detect a soluble polypeptide derived from a protein selected from the list consisting of proteins defined by SEQ ID Nos 1-18 as a marker in one or more samples obtained from said subject; and
(b) correlating the results of said assay(s) to the presence or absence of colorectal cancer in the subject, to a therapeutic regimen to be used in the subject, to a risk of relapse in the subject, or to the prognostic risk of one or more clinical outcomes for the subject suffering from colorectal cancer.

2. A method according to claim 1 wherein the soluble polypeptide detected in step (a) is derived from a protein defined by any one of SEQ ID Nos 4, 7, 8, 13 and 15.

3. A method according to claim 1 wherein step (b) involves determining that when the level of said detected marker is higher in the subject than a control level, said determination indicates the presence of colorectal cancer in the subject, indicates a greater risk of relapse in the subject, or indicates a worse prognosis for the subject.

4. A method according to claim 1 which is a method for diagnosing colorectal cancer in a subject.

5. A method according to claim 1 wherein the marker comprises an amino acid sequence recited in column 4 of Table 1, namely any one of SEQ ID Nos 34-35, 37-38, 40-42, 44, 47-56, 59-60, 62, 64-83, 85-87, 89-92, 95-127, 132-133, 137-141, 144-147, 149, 151-153, 155-161, 164-165, 167-175, 177-179, 182-187, 189-190, 193-195, 197-200, 202, 205-209, 211, 213-227, 229-241, 243.

6. A method according to claim 1 wherein the marker comprises an amino acid sequence recited in column 4 of Table 2, namely any one of SEQ ID Nos 36, 39-40, 42-43, 45-47, 57-58, 61, 63, 66, 75, 84, 88, 91, 93-94, 98, 100, 108, 111, 115, 121, 123-124, 126, 128-131, 134-136, 140, 142-143, 147-150, 152-154, 160-163, 166, 168, 172, 174-176, 180-181, 188, 190-192, 196, 200-201, 203-204, 212, 214, 216, 218, 224, 228, 238-239, 242, 244-245.

7. A method according to claim 1 wherein the marker is derived from a protein in an isoform characterized by a pI and MW as listed in columns 2 and 3 of Table 2.

8. A method according to claim 1 wherein the marker sequence overlaps with or is preferably within a sequence corresponding to an extracellular portion of a protein having a sequence selected from any one of SEQ ID Nos 1-18 (i.e. overlaps with or is preferably within a sequence corresponding to a sequence selected from SEQ ID Nos 19, 21, 22, 25, 27, 29, 30 and 32).

9. A method according to claim 8 wherein the marker sequence overlaps with or is preferably within a sequence corresponding to an extracellular portion of a protein having a sequence selected from any one of SEQ ID Nos 4, 7, 8, 13 and 15.

10. A method according to claim 1, wherein the method comprises performing assays configured to detect two or more said markers.

11. A method according to claim 10 wherein the two or more said markers are derived from at least two different proteins.

12. A method according to claim 1, wherein the method comprises performing assays configured to detect three or more said markers.

13. A method according to claim 12 wherein the three or more said markers are derived from at least three different proteins.

14. A method according to claim 1, wherein the method comprises performing assays configured to detect four or more said markers.

15. A method according to claim 14 wherein the four or more said markers are derived from at least four different proteins.

16. A method according to claim 1, wherein the method comprises performing assays configured to detect five or more said markers.

17. A method according to claim 16 wherein the five or more said markers are derived from at least five different proteins.

18. A method according to claim 1, wherein the method comprises performing one or more additional assays configured to detect one or more additional markers in addition to the soluble polypeptide derived from a protein selected from the list consisting of proteins defined by SEQ ID Nos 1-18 and wherein said correlating step comprises correlating the results of said assay(s) and the results of said additional assay(s) to the presence or absence of colorectal cancer in the subject, to a risk of relapse in the subject, or to the prognostic risk of one or more clinical outcomes for the subject suffering from colorectal cancer.

19. A method according to claim 18 wherein the soluble polypeptide is derived from a protein defined by any one of SEQ ID Nos 4, 7, 8, 13 and 15.

20. A method according to claim 1, wherein the subject is a human.

21. A method according to claim 1, wherein one or more of said assay(s) is an immunoassay.

22. An antibody or other affinity reagent such as an Affibody, Nanobody or Unibody capable of immunospecific binding to a soluble polypeptide derived from a protein selected from the list consisting of proteins defined by SEQ ID Nos 1-18.

23. An antibody according to claim 22 wherein the soluble polypeptide is derived from a protein defined by any one of SEQ ID Nos 4, 7, 8, 13 and 15.

24. A kit comprising an antibody or other affinity reagent such as an Affibody, Nanobody or Unibody as defined in claim 22.

25. A kit comprising a plurality of distinct antibodies or other affinity reagents such as Affibodies, Nanobodies or Unibodies as defined in claim 22.

26. (canceled)

27. A method for identifying the presence or absence of colorectal cancer cells in a biological sample obtained from a human subject, which comprises the step of identifying the presence or absence of one or more soluble polypeptides derived from a protein selected from the list consisting of proteins defined by any one of SEQ ID Nos 1-18.

28. A method according to claim 27 wherein the soluble polypeptide is derived from a protein defined by any one of SEQ ID Nos 4, 7, 8, 13 and 15.

29. A method of detecting, diagnosing colorectal cancer in a subject, differentiating causes of colorectal cancer in a subject, guiding therapy in a subject suffering from colorectal cancer, assessing the risk of relapse in a subject suffering from colorectal cancer, or assigning a prognostic risk of one or more future clinical outcomes to a subject suffering from colorectal cancer, the method comprising:
(a) bringing into contact with a sample to be tested from said subject one or more antibodies, or other affinity reagents such as Affibodies, Nanobodies or Unibodies, capable of specific binding to a soluble polypeptides derived from a protein selected from the list consisting of proteins defined by SEQ ID Nos 1-18; and
(b) thereby detecting the presence of one or more soluble polypeptides derived from a protein selected from the list consisting of proteins defined by any one of SEQ ID Nos 1-18 in the sample.

30. A method according to claim 29 wherein the soluble polypeptide is derived from a protein defined by any one of SEQ ID Nos 4, 7, 8, 13 and 15.

31. A method of detecting colorectal cancer in a patient according to claim 29 wherein the presence of one or more said soluble polypeptides indicates the presence of colorectal cancer in the patient.

32. A method for identifying the presence of colorectal cancer in a subject which comprises the step of carrying out a whole body scan of said subject to determine the localisation of colorectal cancer cells, particularly metastatic colorectal cancer cells, in order to determine presence or amount of one or more soluble polypeptides derived from a protein selected from the list consisting of proteins defined by SEQ ID Nos 1-18, wherein the presence or amount of one or more of said soluble polypeptides indicates the presence of colorectal cancer in the subject.

33. A method according to claim 32 wherein the soluble polypeptide is derived from a protein defined by any one of SEQ ID Nos 4, 7, 8, 13 and 15.

34. A method for identifying the presence of colorectal cancer in a subject which comprises determining the localisation of colorectal cancer cells by reference to a whole body scan of said subject, which scan indicates the presence or amount of one or more soluble polypeptides derived from a protein selected from the list consisting of proteins defined by SEQ ID Nos I-18, wherein the presence or amount of one or more of said soluble polypeptides indicates the presence of colorectal cancer in the subject.

35. A method according to claim 34 wherein the soluble polypeptide is derived from a protein defined by any one of SEQ ID Nos 4, 7, 8, 13 and 15.

36. A method as claimed in claim 32, wherein labelled antibodies, or other affinity reagents such as Affibodies, Nanobodies or Unibodies, are employed to determine the presence of one or more said soluble polypeptides.

37. A diagnostic kit comprising one or more reagents for use in the detection and/or determination of one or more soluble polypeptides derived from a protein selected from the list consisting of proteins defined by SEQ ID Nos 1-18.

38. A kit as claimed in claim 37 wherein the soluble polypeptide is particularly derived from a protein defined by any one of SEQ ID Nos 4, 7, 8, 13 and 15.

39. A kit as claimed in claim 37, which comprises one or more containers with one or more antibodies, or other affinity reagents such as Affibodies, Nanobodies or Unibodies, against one or more said soluble polypeptides.

40. A kit as claimed in claim 39, which further comprises a labelled binding partner to the or each antibody, or other affinity reagent such as an Affibody, Nanobody or Unibody, and/or a solid phase, such as a reagent strip, upon which the or each antibody, or other affinity reagent such as an Affibody, Nanobody or Unibody, is/are immobilised.

41. A method of detecting, diagnosing colorectal cancer in a subject, differentiating causes of colorectal cancer in a subject, guiding therapy in a subject suffering from colorectal cancer, assessing the risk of relapse in a subject suffering from colorectal cancer, or assigning a prognostic risk of one or more future clinical outcomes to a subject suffering from colorectal cancer, the method comprising:
(a) bringing into contact with a sample to be tested one or more soluble polypeptides derived from a protein selected from the list consisting of proteins defined by SEQ ID Nos 1-18 or one or more antigenic or immunogenic fragments thereof; and
(b) detecting the presence of antibodies, or other affinity reagents such as Affibodies, Nanobodies or Unibodies, in the subject capable of specific binding to one or more of said polypeptides, or antigenic or immunogenic fragments thereof.

42. A method according to claim 41 wherein the soluble polypeptide is derived from a protein defined by any one of SEQ ID Nos 4, 7, 8, 13 and 15.

43. A kit for use in the detection, diagnosis of colorectal cancer in a subject, for differentiating causes of colorectal cancer in a subject, for guiding therapy in a subject suffering from colorectal cancer, for assessing the risk of relapse in a subject suffering from colorectal cancer, or for assigning a prognostic risk of one or more future clinical outcomes to a subject suffering from colorectal cancer, which kit comprises one or more soluble polypeptides derived from a protein selected from the list consisting of proteins defined by SEQ ID Nos 1-18 and/or one or more antigenic or immunogenic fragments thereof.

44. A kit as claimed in claim 43 wherein the soluble polypeptide is derived from a protein defined by any one of SEQ ID Nos 4, 7, 8, 13 and 15.

45. A vaccine comprising one or more soluble polypeptides derived from a protein selected from the list consisting of proteins defined by SEQ ID Nos 1-18 and/or one or more antigenic or immunogenic fragments thereof.

46. A vaccine as claimed in claim 45 wherein the soluble polypeptide is derived from a protein defined by any one of SEQ ID Nos 4, 7, 8, 13 and 15.

47. An immunogenic composition which comprises one or more soluble polypeptides derived from a protein selected from the list consisting of proteins defined by SEQ ID Nos 1-18 and/or one or more antigenic or immunogenic fragments thereof, and one or more suitable adjuvants.

48. An immunogenic composition as claimed in claim 47 wherein the soluble polypeptide is derived from a protein defined by any one of SEQ ID Nos 4, 7, 8, 13 and 15.

49. (canceled)

50. (canceled)

51. (canceled)

52. A method for the treatment or prophylaxis of colorectal cancer in a subject, or of vaccinating a subject against colorectal cancer, which comprises the step of administering to the subject an effective amount of one or more soluble polypeptides derived from a protein selected from the list consisting of proteins defined by SEQ ID Nos 1-18 and/or one or more antigenic or immunogenic fragments thereof, preferably as a vaccine.

53. A method according to claim 52 wherein the soluble polypeptide is derived from a protein defined by any one of SEQ ID Nos 4, 7, 8, 13 and 15.

54. A method according to claim 1 wherein the soluble polypeptide derived from a protein selected from the list consisting of proteins defined by SEQ ID Nos 1-18, is detected by a method which involves use of an imaging technology.

55. A method according to claim 54 wherein the imaging technology involves use of labelled Affibodies.

56. A method according to claim 54 wherein the imaging technology involves use of labelled antibodies.

57. A method for identifying the presence of colorectal cancer in a subject which comprises the step of carrying out immunohistochemistry to determine the localisation of colorectal cancer cells, particularly metastatic colorectal cancer cells, in tissue sections, by the use of labeled antibodies, or other affinity reagents such as Affibodies, Nanobodies or Unibodies, derivatives and analogs thereof, capable of specific binding to one or more soluble polypeptides derived from a protein selected from the list consisting of proteins defined by SEQ ID Nos 1-18 or one or more antigenic or immunogenic fragments thereof, in order to determine presence or amount of one or more of said soluble polypeptides, wherein the presence or amount of one or more of said soluble polypeptides indicates the presence of colorectal cancer in the subject.

58. A method according to claim 57 wherein the soluble polypeptide is derived from a protein defined by any one of SEQ ID Nos 4, 7, 8, 13 and 15.
